(19) United States
(12) Patent Application Publication
(10) Pub. No.: US 2009/0318527 A1
(43) Pub. Date: Dec. 24, 2009

(54) INDOLE COMPOUNDS AND METHODS OF USE THEREOF

(76) Inventors: Howard Sard, Arlington, MA (US); Yiliang Zhang, Lowell, MA (US); Jie Li, Baltimore, MD (US); Louis Shuster, Brookline, MA (US); Bryan Roth, Durham, NC (US); Niels Jensen, Carrboro, NC (US)

Correspondence Address: LANDO & ANASTASI, LLP, ONE MAIN STREET, SUITE 1100, CAMBRIDGE, MA, 02142, US

(21) Appl. No.: 12/369,659

(22) Filed: Feb. 11, 2009

Related U.S. Application Data
(60) Provisional application No. 61/027,825, filed on Feb. 11, 2008.

(51) Int. Cl. A61K 31/404; C07D 209/14

(52) U.S. Cl. 514/415; 548/491

(57) ABSTRACT

Novel indole compounds are disclosed. Also disclosed are methods for using the compounds to treat human and animal disease, pharmaceutical compositions of the compounds, and kits including the compounds.

### RELATED APPLICATIONS

The present application claims priority under 35 U.S.C. § 119(e) to U.S. provisional application U.S. Ser. No. 61/027,825, filed Feb. 11, 2008, which is incorporated herein by reference.

### STATEMENT OF GOVERNMENT INTEREST

The invention was made at least in part with National Institutes of Health grant R44MH063529. The U.S. Government may have certain rights in the invention.

### FIELD OF THE INVENTION

This invention relates to novel indole compounds and their use as selective agents at serotonin receptors.

### BACKGROUND OF THE INVENTION

Serotonin (5-hydroxytryptamine, 5-HT) plays a significant role in influencing a large number of central and peripheral processes. 5-HT-selective pharmacotherapies have been developed to treat a wide variety of medical problems including depression, anxiety, schizophrenia, migraine, emesis, and appetite control (Annual Reports in Medicinal Chemistry, Volume 32, 2002, Academic Press, Fitzgerald, L., Ennis, M. “5-HT_{2C} Receptor Modulators: Progress in Development of New CNS Medicines” pp 21-30). 5-HT exerts its influence through activation of fourteen distinct receptor subtypes in seven separate families. There is particular interest in the three receptor subtypes of the 5-HT₂ family, 5-HT_{2A}, 5-HT_{2B}, and 5-HT_{2C}. Modulation of the 5-HT_{2C} receptor subtype has been shown to play a role in numerous human diseases including obesity, obsessive-compulsive disorder (OCD), sexual dysfunction, epilepsy, schizophrenia, and anxiety disorders (Roth, B., Shapiro, D. “Insights into the Structure and Function of 5-HT2 Family Serotonin Receptors Reveal Novel Strategies for Therapeutic Target Development” Expert Opin. Ther. Targets 2001, 5, 685; Martin, J., Bos, M., Jenck, F., Moreau, J-1., Mutel, V., Sleight, A., Wichmann, J., Andrews, J., Berendsen, H., Broekkamp, C., Ruight, G., Kohler, C., van Delft, A. “5-HT_{2C} Receptor Agonists: Pharmacological Characteristics and Therapeutic Potential” J. Pharm. Experimental Ther. 1998, 286, 913). However, the transmembrane sequence homology between the 5-HT_{2C} receptor and the 5-HT_{2A} and 5-HT_{2B} receptors is high (Bickerdike, M., Vickers, S., Dourish, C. “5-HT_{2C} Receptor Modulation and the Treatment of Obesity” Diabetes Obes. Metab. 1999, 1, 207; Glennon, R., Dukat, M., El-Bermawy, M., Law, H., De Los Angeles, J., Teitler, M., King, A., Herrick-Davis, K. “Influence of Amine Substituents on 5-HT_{2A} versus 5-HT_{2C} Binding of Phenylalkyl- and Indolylalkylamines” J. Med. Chem. 1994, 37, 1929). Thus selectivity for the 5-HT_{2C} receptor can be difficult to obtain, however such selectivity is important from a drug development standpoint. 5-HT_{2B} receptor agonists are associated with heart valve toxicity (Rothman, R., Baumann, M., Savage, J., Rauser, L., McBride, A., Hufeisen, S., Roth, B. L. “Evidence for Possible Involvement of 5-HT_{2B} Receptors in the Cardiac Valvulopathy Associated with Fenfluramine and other Serotonergic Medications” Circulation 2000, 102, 2836; Fitzgerald, L., Burn, T., Brown, B., Patterson, J., Corjay, M., Valentine, P., Sun, J-H., Link, J., Abbaszade, I., Hollis, J., Largent, B., Hartig, P., Hollis, G., Meunier, P., Robichaud, A., Robertson, D. “Possible Role of Valvular Serotonin 5-HT_{2B} Receptors in the Cardiopathy Associated with Fenfluramine” Molecular Pharmacology 2000, 57, 75) and pulmonary hypertension (Launay, J., Herve, P., Peoc'h, K., Tournois, C., Callebert, J., Nebigil, C., Etienne, N., Drouet, L., Humbert, M., Simonneau, G., Maroteaux, L. “Function of the Serotonin 5-Hydroxytryptamine 2B Receptor in Pulmonary Hypertension” Nat. Med. 2002, 8, 1129). However, the 5-HT_{2C} receptor is found only in the CNS (Bickerdike, M., Vickers, S., Dourish, C. “5-HT_{2C} Receptor Modulation and the Treatment of Obesity” Diabetes Obes. Metab. 1999, 1, 207; Martin, J., Bos, M., Jenck, F., Moreau, J-1., Mutel, V., Sleight, A., Wichmann, J., Andrews, J., Berendsen, H., Broekkamp, C., Ruight, G., Kohler, C., van Delft, A. “5-HT_{2C} Receptor Agonists: Pharmacological Characteristics and Therapeutic Potential” J. Pharm. Experimental Ther. 1998, 286, 913), and agonists that discriminate for 5-HT_{2C} over 5-HT_{2B} should not display cardio- or pulmonary toxicity. Selectivity for 5-HT_{2C} over 5-HT_{2A} receptors is also important since agonists at 5-HT_{2A} generally display undesirable hallucinogenic activity (e.g. LSD, psilocybin).

Psilocybin (4-phosphoryloxy-N,N-dimethyltryptamine) is an agonist at the 5-HT_{2A} and 5-HT_{2C} receptors. Its binding potency at 5-HT_{2A} correlates with its activity as a hallucinogen in humans (Delgado, P. L., Moreno, F. A. “Hallucinogens, Serotonin, and Obsessive-Compulsive Disorder” J. Psychoactive Drugs 1998, 30, 359; Perrine, D. M. “Hallucinogens and Obsessive-Compulsive Disorder” Am. J. Psychiatry 1999, 156, 1123; Moreno, F. A., Delgado, P. L. “Hallucinogen-Induced Relief of Obsessions and Compulsions” Am. J. Psychiatry 1997, 154, 1037). More than 40 years ago, some derivatives of psilocybin were reported by workers at Sandoz (Hofmann, A., Troxler, F. U.S. Pat. No. 3,075,992; U.S. Pat. No. 3,078,214). This work was carried out prior to the ability to test for activity at specific serotonin receptor subtypes. More recently, considerable effort has been made in seeking selective 5-HT_{2C} receptor ligands. The indole Ro 60-0175 (Martin, J., Bos, M., Jenck, F., Moreau, J-, Mutel, V., Sleight, A., Wichmann, J., Andrews, J., Berendsen, H., Broekkamp, C., Ruight, G., Kohler, C., van Delft, A. “5-HT_{2C} Receptor Agonists: Pharmacological Characteristics and Therapeutic Potential” J. Pharm. Experimental Ther. 1998, 286, 913; Bos, M., Jenck, F., Martin, J., Moreau, J-l., Sleight, A., Wichmann, J., Widmer, U. “Novel Agonists of 5-HT_{2C} Receptors. Synthesis and Biological Evaluation of Substituted 2-(Indol-1-yl)-1-methylethylamines and 2-(Indeno[1,2-b]pyrrol-1-methylethylamines. Improved Therapeutics for Obsessive Compulsive Disorder” J. Med. Chem. 1997, 45, 2762) is 25 times more active at 5-HT_{2C} as compared to 5-HT_{2A}, however it is not selective over 5-HT_{2B} receptors (Bickerdike, M., Vickers, S., Dourish, C. “5-HT_{2C} Receptor Modulation and the Treatment of Obesity” Diabetes Obes. Metab. 1999, 1, 207). Certain 1-methyl-5-substituted indoles reported by Lilly are selective 5-HT_{2B} antagonists (Audia, J., Evrard, D., Murdoch, G., Droste, J., Nissen, J., Schenck, K., Fludzinski, Z., Lucaites, V., Nelson, D., Cohen, M. “Potent, Selective Tetrahydro-β-carboline Antagonists of the Serotonin 2B (5HT_{2B}) Contractile Receptor in the Rat Stomach Fundus” J. Med. Chem. 1996, 39, 2773). Some N-1-substituted 6-methoxyindazoles are selective 5-HT_{2C} agonists (May, J., Dantanarayana, A., Zinke, P., McLaughlin, M., Sharif, N. “1-((S)-2-Aminopropyl-1H-6-indazol-6-ol: A Potent Peripherally Acting 5-HT2 Receptor Agonist with Ocular Hypotensive Activity” J. Med. Chem. 2006, 49, 318). Substituted indoles were reported by Vernalis to be highly selective agonists at 5-HT_{2C} as compared to 5-HT_{2A} (American Chemical Society National Meeting, Boston, Mass., Aug. 18-22, 2002, Poster Session, Division of Medicinal Chemistry, Wednesday morning, August 21, #344-349). Wyeth has reported 5-HT_{2C} agonists including WAY161503 that are active in an animal model of obesity (Welmaker, G., Nelson, A., Sabalski, J., Sabb, A., Potoski, J., Graziano, D., Kagan, M., Coupet, J., Dunlop, J., Mazandarani, H., Rosenzweig-Lipson, S., Sukoff, S., Zhang, Y. “Synthesis and 5-hydroxytryptamine (5-HT) activity of 2,3,4,4a-tetrahydro-1H-pyrazino[1,2-a]quinoxalin-5-(6H)ones and 2,3,4,4a,5,6-hexahydro-1H-pyrazino[1,2-a]quinoxalines” Bioorg. Med. Chem. Lett. 2000, 10, 1991). Yamanouchi has described indazole compounds including YM348 which are 5-HT_{2C} agonists showing activity in an animal model of obesity (Kimura, Y., Hatanaka, K., Naitou, Y., Maeno, K., Shimada, I., Koakutsu, A., Wanibuchi, F., Yamaguchi, T., “Pharmacological profile of YM348, a novel, potent and orally active 5-HT_{2C} receptor agonist” Eur. J. Pharmacol. 2004, 483, 37). Arena reports mCPP analogs active in vivo as potential obesity treatments (Smith, B., Smith, J., Tsai, J., Schultz, J., Gilson, C., Estrada, S., Chen, R., Park, D., Prieto, E., Gallardo, C., Sengupta, D., Thomsen, W., Saldana, H., Whelan, K., Menzaghi, F., Webb. R., Beeley, N. “Discovery and SAR of new benzazepines as potent and selective 5-HT(2C) receptor agonists for the treatment of obesity” Bioorg. Med. Chem. Lett. 2005, 15, 1467) and one of these 5-HT_{2C} agonists, APD-356 (lorcaserin), is under clinical development as an appetite suppressant.

Obesity is one of the most important health problems currently affecting the U.S. population. The overweight suffer a significantly higher death rate, as well as a much greater risk of developing many diseases including type 2 diabetes, sleep apnea, hypertension, osteoarthritis, and some forms of cancer. Exercise and diet modification allow some obese people to lose weight. However, many others are unable to achieve lasting weight loss by such methods, and pharmaceutical agents that promote satiety can be effective and appropriate treatments.

Considerable evidence has accumulated implicating 5-HT_{2C} receptor activation with appetite suppression. In 1995, transgenic mice lacking the 5-HT_{2C} receptor were shown to become obese (Tecott, L., Sun, L., Akana, S., Strack, A., Lowenstein, D., Dallman, M., Julius, D. “Eating Disorder and Epilepsy in Mice Lacking 5-HT_{2C} Serotonin Receptors” Nature 1995, 374, 542). A clinical study in 1997 (Sargent, P., Sharpley, A., Williams, C., Cowen, P. “5-HT_{2C}-Receptor Activation Decreases Appetite and Body Weight in Obese Subjects” Psychopharmacology 1997, 133, 309) using meta-chlorophenylpiperazine (mCPP), a 5-HT_{2C} agonist, has shown appetite reduction and weight loss in obese subjects. Furthermore, selective 5-HT_{2C} antagonists reduce or eliminate the anorexic effects of 5-HT_{2C} agonists (Kennett, G., Wood, M., Bright, F., Trail, B., Riley, G., Holland, K., Avenell, K., Stean, T., Upton, N., Bromidge, S., Forbes, I., Middlemiss, D., Blackburn, T. “SB242082, a Selective and Brain Penetrant 5-HT_{2C} Receptor Antagonist” Neuropharmacology, 1997, 36, 609). Fenfluramine is a non-selective 5-HT_{2C} receptor agonist which together with phenteramine (“phen-fen”) was marketed until recently as a highly effective appetite suppressant. The clinical effectiveness of fenfluramine as an appetite suppressant has been shown to be largely due to its activity as a 5-HT_{2C} receptor agonist (Bickerdike, M., Vickers, S., Dourish, C. “5-HT_{2C} Receptor Modulation and the Treatment of Obesity” Diabetes Obes. Metab. 1999, 1, 207; Vickers, S., Dourish, C., Kennett, G. “Evidence that Hypophagia Induced by d-Fenfluramine and d-Norfenfluramine in the Rat is Mediated by 5-HT_{2C} Receptors” Neuropharmacology 2001, 41, 200).

Obsessive Compulsive Disorder (OCD) is a mental illness involving persistent and distressing thoughts and actions that significantly interfere with normal life. OCD afflicts at least 1-2% of the population in the US and worldwide, and is the fourth most common psychiatric diagnosis in the United States (Delgado, P. L., Moreno, F. A. “Hallucinogens, Serotonin, and Obsessive-Compulsive Disorder” J. Psychoactive Drugs 1998, 30, 359; Goodman, W. K. “Obsessive-Compulsive Disorder: Diagnosis and Treatment” J. Clin. Psychiatry 1999, 60 (Suppl 18), 27). OCD is currently treated pharmacologically and/or with psychotherapy. Current pharmacotherapy for OCD has significant limitations and the discovery of an improved medication for OCD would have considerable commercial potential.

Psilocybin, a 5-HT_{2C} receptor agonist, has undergone a Phase I clinical trial with OCD patients (Moreno, F.; Wiegand, C.; Taitano, E.; Delgado, P. “Safety, Tolerability, and Efficacy of Psilocybin in 9 Patients with Obsessive-Compulsive Disorder” J. Clin. Psychiatry 2006, 67, 1735). Other 5-HT_{2C} receptor agonists are recognized as potential treatments for OCD (Martin, J., Bos, M., Jenck, F., Moreau, J-l., Mutel, V., Sleight, A., Wichmann, J., Andrews, J., Berendsen, H., Broekkamp, C., Ruight, G., Kohler, C., van Delft, A. “5-HT_{2C} Receptor Agonists Pharmacological Characteristics and Therapeutic Potential” J. Pharm. Experimental Ther. 1998, 286, 913).

Other possible uses for 5-HT_{2C} selective compounds include treatments for schizophrenia and psychosis (Ramamoorthy, P. “[1,4]Diazepino[6,7-ij]quinoline derivatives as antipsychotic and antiobesity agents” U.S. Patent Application US2004/0009970 A1, Jan. 15, 2004 (Wyeth); Dunlop, J., Marquis, K., Lim, H., Leung, L., Kao, J., Cheesman, C., Rosenzweig-Lipson, S. “Pharmacological Profile of the 5-HT_{2C} Receptor Agonist WAY-163909; Therapeutic Potential in Multiple Indications” CNS Drug Reviews 2006, 12, 167); anxiety and depression (Heisler, L., Zhou, L., Bajwa, J., Hsu, J., Tecott, L. “Serotonin 5-HT_{2C} Receptors Regulate Anxiety-like Behavior” Genes, Brain, and Behavior 2007, 6, 491); diabetes (Zhou, L., Sutton, G., Rochford, J., Semple, R., Lam, D., Oksanen, L., Thornton-Jones, Z., Clifton, P., Yueh, C.-Y., Evans, M., McCrimmon, R., Elmquist, J., Butler, A, Heisler, L. “Serotonin 2C Receptor Agonists Improve Type 2 Diabetes via Melanocortin-4 Receptor Signaling Pathways” Cell Metabolism 2007, 6, 398); epilepsy (Isaac, M. “Serotonergic 5-HT_{2C} Receptors as a Potential Therapeutic Target for the Design Antiepileptic Drugs” Current Topics in Medicinal Chemistry 2005, 5, 59); Alzheimer's disease (Arjona, A., Pooler, A., Lee, R., Wurtman, R. “Effect of a 5-HT(2C) Serotonin Agonist, Dexnorfenfluramine, on Amyloid Precursor Protein Metabolism in Guinea Pigs” Brain Res. 2002, 951, 135), sexual dysfunction (Uckert, S., Stief, C., Jonas, U. “Current and Future Trends in the Oral Pharmacotherapy of Male Erectile Dysfunction” Expert Opin. Investig. Drugs 2003, 12, 1521; Millan, M., Peglion, J., Lavielle, G., Perrin-Monneyron, S. “5-HT2C Receptors Mediate Penile Erections in Rats: Actions of Novel and Selective Agonists and Antagonists” Eur. J. Pharmacol. 1997, 325, 9), and substance abuse and addiction disorders (Kampman, K., Pettinata, H., Lynch, K., Sparkman, T., O'Brien, C “A Pilot Trial of Olanzapine for Cocaine Dependence” Drug Alcohol Depend. 2003, 70, 265; Fletcher, P., Rizos, Z., Sinyard, J., Tampakeras, M., Higgins, G. “The 5HT(2C) Receptor Agonist Ro60-0175 Reduces Cocaine Self-Administration and Reinstatement Induced by the Stressor Yohimbine, and Contextual Cues” Neuropsychopharmacology 2007, online publication Jul. 25, 2007, doi:10.1038/sj.npp.1301509).

Compounds that are selective for the 5-HT_{2C} receptor may therefore have therapeutic potential in treating, for example, the above disorders. Such selectivity can also reduce possible side effects due to activity at other serotonin receptors.

Certain N—H psilocin derivatives containing fluorine substitution at the 5-, 6-, or 7-position have been reported (Blair, J., Kurrasch-Orbaugh, D., Marona-Lewicka, D., Cumbay, M., Watts, V., Barker, E., Nichols, D. “Effect of Fluorine Substitution on the Pharmacology of Hallucinogenic Tryptamines” J. Med. Chem. 2000, 43, 4701), and some of these compounds were shown to have reduced activity at the 5-HT_{2A} receptor as compared to psilocin itself.

### SUMMARY OF THE INVENTION

The present invention relates to novel N-substituted psilocin (4-hydroxyindole) derivatives that are also substituted at the 5-, 6-, and/or 7-positions and possess 5-HT_{2C} receptor selectivity, preferably versus both the 5HT_{2A} and 5-HT_{2B} receptors. Such compounds have not previously been described or recognized to have selective functional activity at the 5-HT_{2C}-receptor or to have in vivo activity in an animal model of human disease.

In one aspect, the invention features a compound represented by the structural formula I:

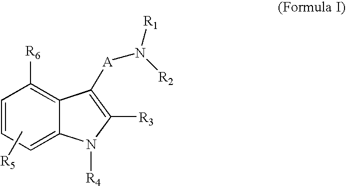

in which

A is C₁-C₄ alkylene, C₂-C₄ alkenylene, or C₂-C₄ alkynylene;

R₁ and R₂ are, independently for each occurrence, H, C₁-C₈ alkyl, C₂-C₈ alkenyl, or C₂-C₈ alkynyl, wherein the C₁-C₈ alkyl, C₂-C₈ alkenyl, or C₂-C₈ alkynyl are optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH;

R₃ is H, C₁-C₈ alkyl, C₂-C₈ alkenyl, or C₂-C₈ alkynyl, wherein the C₁-C₈ alkyl, C₂-C₈ alkenyl, or C₂-C₈ alkynyl are optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH; or R₃ is selected from the group consisting of halogen, C₁-C₈ alkylsulfonyl, formyl, COOH, hydroxy, C₁-C₈ alkoxyl, —SH, thio(C₂-C₈)alkyl, amino, C₁-C₈ alkylamino, and di(C₁-C₈ alkyl)amino;

R₄ is C₁-C₈ alkyl, C₂-C₈ alkenyl, or C₂-C₈ alkynyl, each of which is optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH; or R₄ is selected from the group consisting of C₁-C₈ alkylsulfonyl, formyl, hydroxy, C₁-C₈ alkoxyl, —SH, thio(C₂-C₈)alkyl, amino, C₁-C₈ alkylamino, and di(C₁-C₈ alkyl)amino;

R₅ represents 1-3 substituents, each of which is independently selected from the group consisting of C₁-C₈ alkyl, C₂-C₈ alkenyl, and C₂-C₈ alkynyl, each of which is optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, and di(C₁-C₈ alkyl)amino; or

R₅ represents 1-3 substituents, each of which is independently selected from the group consisting of halogen, C₁-C₈ alkylsulfonyl, formyl, COOH, hydroxy, C₁-C₈ alkoxyl, —SH, thio(C₂-C₈)alkyl, amino, C₁-C₈ alkylamino, and di(C₁-C₈ alkyl)amino;

R₆ is F or OR₇; and

R₇ is C₁-C₈ alkyl, optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH;

or a pharmaceutically acceptable salt thereof.

In some embodiments, A is C₁-C₄ alkylene, e.g., C₂ alkylene.

In some embodiments, R₁ is hydrogen.

In some embodiments, R₁ is C₁-C₈ alkyl, e.g., C₁ alkyl or C₂ alkyl. In some embodiments, R₁ is C₁-C₈ alkyl substituted with 1-3 substituents. In some embodiments, R₁ is C₁-C₈ alkyl substituted with 1-3 substituents, wherein at least one substituent is halogen (e.g., fluorine). In some embodiments, R₁ is C₂ alkyl substituted with 1-3 substituents, e.g., 1-3 halogens, e.g., 1-3 fluorines. In some embodiments, R₁ is C₂ alkyl substituted with 3 fluorines. In some embodiments, R₁ is —CH₂—CF₃.

In some embodiments, R¹ is C₁-C₈ alkyl, C₂-C₈ alkenyl, or C₂-C₈ alkynyl, wherein each carbon of the C₁-C₈ alkyl, C₂-C₈ alkenyl, or C₂-C₈ alkynyl is substituted with no more than 2 substituents, each of which is independently selected from the group consisting of cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH.

In some embodiments, R₂ is hydrogen. In some embodiments, R₂ is C₁-C₈ alkyl, e.g., C₁ alkyl or C₂ alkyl. In some embodiments, R₂ is C₁-C₈ alkyl substituted with 1-3 substituents. In some embodiments, R₂ is C₁-C₈ alkyl substituted with 1-3 substituents, wherein at least one substituent is halogen (e.g., fluorine). In some embodiments, R₂ is C₂ alkyl substituted with 1-3 substituents, e.g., 1-3 halogens, e.g., 1-3 fluorines. In some embodiments, C₂ alkyl substituted with 3 fluorines. In some embodiments, R₂ is —CH₂—CF₃.

In some embodiments, R² is C₁-C₈ alkyl, C₂-C₈ alkenyl, or C₂-C₈ alkynyl, wherein each carbon of the C₁-C₈ alkyl, C₂-C₈ alkenyl, or C₂-C₈ alkynyl is substituted with no more than 2 substituents, each of which is independently selected from the group consisting of cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH.

In some embodiments, R₁ and R₂ are both C₁-C₈ alkyl, e.g., C₁ alkyl or C₂ alkyl. In some embodiments, R₁ is C₁ alkyl and R₂ is C₂ alkyl. In some embodiments, both R₁ and R₂ are C₁-C₈ alkyl substituted with 1-3 substituents. In some embodiments, both R₁ and R₂ are C₁-C₈ alkyl substituted with 1-3 substituents, wherein at least one substituent is halogen (e.g., fluorine).

In some embodiments, R₁ and R₂ are C₂ alkyl substituted with 1-3 substituents, e.g., 1-3 halogens, e.g., 1-3 fluorines. In some embodiments, both R₁ and R₂ are C₂ alkyl substituted with 3 fluorines. In some embodiments, both R₁ and R₂ are —CH₂—CF₃.

In some embodiments, R₁ is hydrogen and R₂ is C₁-C₈ alkyl (e.g., C₁ alkyl or C₂ alkyl).

In some embodiments, R₁ and R₂, together with the nitrogen to which they are attached, form a group selected from the following:

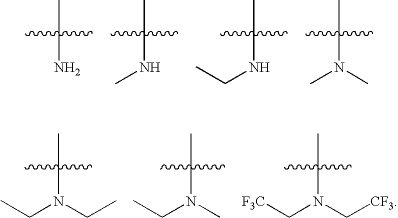

In some embodiments, R₃ is hydrogen.

In some embodiments, R³ is C₁-C₈ alkyl, C₂-C₈ alkenyl, or C₂-C₈ alkynyl, wherein each carbon of the C₁-C₈ alkyl, C₂-C₈ alkenyl, or C₂-C₈ alkynyl is substituted with no more than 2 substituents, each of which is independently selected from the group consisting of cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH.

In some embodiments, R₄ is C₁-C₈ alkyl, C₂-C₈ alkenyl, or C₂-C₈ alkynyl, each of which is optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH; or R₄ is selected from the group consisting of C₁-C₈ alkylsulfonyl, formyl, hydroxy, C₁-C₈ alkoxyl, and thio(C₂-C₈)alkyl.

In some embodiments, R⁴ is C₁-C₈ alkyl, C₂-C₈ alkenyl, or C₂-C₈ alkynyl, wherein each carbon of the C₁-C₈ alkyl, C₂-C₈ alkenyl, or C₂-C₈ alkynyl is substituted with no more than 2 substituents, each of which is independently selected from the group consisting of cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH.

In some embodiments, R₄ is C₁-C₈ alkyl, e.g., C₁, C₂, C₃ or C₄ alkyl. In some embodiments, R₄ is cycloalkyl, e.g., C₃, C₄, C₅, C₆ or C₇ cycloalkyl. In some embodiments, R₄ is cycloalkyl substituted alkyl. In some embodiments, R₄ is cycloalkyl substituted C₁-C₄ alkyl (e.g., cycloalkyl substituted C₁, C₂, C₃ or C₄ alkyl).

In some embodiments, R₅ represents 1 substituent.

In some embodiments, the compound is of the following formula:

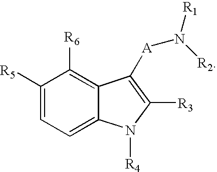

In some embodiments, R₅ is halogen, e.g., fluorine.

In some embodiments, the compound is of the following formula:

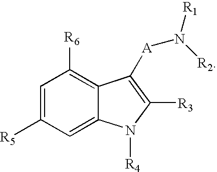

In some embodiments, R₅ is halogen, e.g., fluorine.

In some embodiments, the compound is of the following formula:

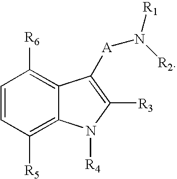

In some embodiments, R₅ is halogen, e.g., fluorine.

In some embodiments, R₅ represents 2 substituents.

In some embodiments, the compound is of the following formula:

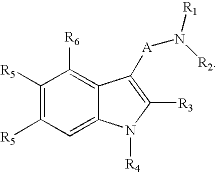

In some embodiments, both R₅ substituents are halogen, e.g., both R₅ substituents are fluorine.

In some embodiments, the compound is of the following formula:

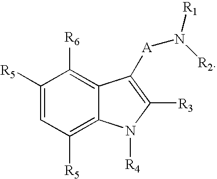

In some embodiments, both R₅ substituents are halogen, e.g., both R₅ substituents are fluorine.

In some embodiments, the compound is of the following formula:

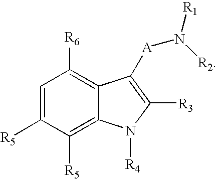

In some embodiments, both R₅ substituents are halogen, e.g., both R₅ substituents are fluorine.

In some embodiments, R⁵ represents 1-3 substituents, each of which is independently selected from the group consisting of C₁-C₈ alkyl, C₂-C₈ alkenyl, or C₂-C₈ alkynyl, wherein each carbon of the C₁-C₈ alkyl, C₂-C₈ alkenyl, or C₂-C₈ alkynyl is substituted with no more than 2 substituents, each of which is independently selected from the group consisting of cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH.

In some embodiments, R₆ is fluorine.

In some embodiments, R₆ is OR₇. In some embodiments, R₇ is C₁-C₈ alkyl, e.g., C₁ alkyl. In some embodiments, R₇ is C₁ alkyl substituted with 1-3 substituents. In some embodiments, R₇ is C₁ alkyl substituted with 1-3 halogens (e.g., 1-3 fluorines). In some embodiments, R₇ is C₁ alkyl substituted with 2 fluorines. In some embodiments, R₇ is C₁ alkyl substituted with 3 fluorines.

In some embodiments, R₇ is C₂ alkyl. In some embodiments, R₇ is C₂ alkyl substituted with 1-3 substituents. In some embodiments, R₇ is C₂ alkyl substituted with 1 substituent. In some embodiments, R₇ is —CH₂—CH₂—OH. In some embodiments, R₇ is —CH₂—CH₂—(C₁-C₈ alkoxyl) (e.g., R₇ is —CH₂—CH₂—O—CH₃). In some embodiments, R₇ is —CH₂—CH₂— (di(C₁-C₈ alkyl)amino) (e.g., R₇ is —CH₂—CH₂—N(CH₃)₂).

In some embodiments, R₇ is C₃ alkyl.

In some embodiments, R₇ is cycloalkyl, e.g., C₃, C₄, C₅, C₆ or C₇ cycloalkyl. In some embodiments, R₇ is cycloalkyl substituted alkyl. In some embodiments, R₇ is cycloalkyl substituted C₁-C₄ alkyl (e.g., cycloalkyl substituted C₁, C₂, C₃ or C₄ alkyl).

In some embodiments, R⁷ is C₁-C₈ alkyl, wherein each carbon of the C₁-C₈ alkyl is substituted with no more than 2 substituents, each of which is independently selected from the group consisting of cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH.

In one aspect, the invention features a pharmaceutical composition comprising a compound of formula I.

In one aspect, the invention features a dosage form comprising a compound of formula I. In some embodiments, the dosage form is an oral dosage form.

In one aspect, the invention features a method for the treatment of obesity in a subject, the method comprising administering to the subject a compound of formula I, such that obesity is treated.

In one aspect, the invention features a method for the treatment of Obsessive Compulsive Disorder (OCD) in a subject, the method comprising administering to the subject a compound of formula I, such that OCD is treated.

In one aspect, the invention features a method for suppressing appetite in a subject, the method comprising administering to the subject a compound of formula I, such that appetite is suppressed in the subject.

In one aspect, the invention features a method for the treatment of schizophrenia or psychosis in a subject, the method comprising administering to the subject a compound of formula I, such that schizophrenia or psychosis is treated.

In one aspect, the invention features A method for the treatment of anxiety or depression in a subject, the method comprising administering to the subject a compound of formula I, such that anxiety or depression is treated in the subject.

In one aspect, the invention features A method for the treatment of diabetes in a subject, the method comprising administering to the subject a compound of formula I, such that diabetes is treated in the subject.

In one aspect, the invention features A method for the treatment of attention deficit hyperactivity disorder (ADHD) in a subject, the method comprising administering to the subject a compound of formula I, such that ADHD is treated in the subject.

In one aspect, the invention features A method for the treatment of suicidal behavior in a subject, the method comprising administering to the subject a compound of formula I, such that suicidal behavior is treated in the subject.

In one aspect, the invention features A method for the treatment of migraine in a subject, the method comprising administering to the subject a compound of formula I, such that migraine is treated in the subject.

In one aspect, the invention features a method for enhancing cognition in a subject, the method comprising administering to the subject a compound of formula I, such that cognition is enhanced in the subject.

In one aspect, the invention features a method for the treatment of a central nervous system disorder in a subject, the method comprising administering to the subject a compound of formula I, such that the central nervous system disorder is treated. In some embodiments, the central nervous system disorder is selected from the group consisting of epilepsy, Alzheimer's disease, sexual dysfunction, addiction, anorexia nervosa, Tourette's syndrome, and trichotillomania.

In one aspect, the invention features a method for the treatment of acral lick dermatitis (ALD) in a canine subject, the method comprising administering to the subject a compound of formula I, such that acral lick dermatitis is treated.

In one aspect, the invention features a method of increasing the activity of a serotonin receptor, the method comprising contacting a serotonin receptor with a compound of formula I. In some embodiments, the serotonin receptor is a 5-HT_{2C} receptor.

In one aspect, the invention features a compound represented by the structural formula II:

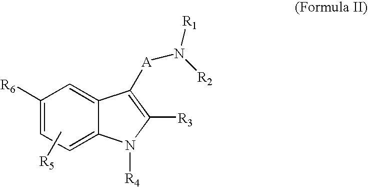

in which

A is C₁-C₄ alkylene, C₂-C₄ alkenylene, or C₂-C₄ alkynylene;

R₁ and R₂ are, independently for each occurrence, H, C₁-C₈ alkyl, C₂-C₈ alkenyl, or C₂-C₈ alkynyl, wherein the C₁-C₈ alkyl, C₂-C₈ alkenyl, or C₂-C₈ alkynyl are optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH;

R₃ is H, C₁-C₈ alkyl, C₂-C₈ alkenyl, or C₂-C₈ alkynyl, wherein the C₁-C₈ alkyl, C₂-C₈ alkenyl, or C₂-C₈ alkynyl are optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH; or R₃ is selected from the group consisting of halogen, C₁-C₈ alkylsulfonyl, formyl, COOH, hydroxy, C₁-C₈ alkoxyl, —SH, thio(C₂-C₈)alkyl, amino, C₁-C₈ alkylamino, and di(C₁-C₈ alkyl)amino;

R₄ is C₁-C₈ alkyl, C₂-C₈ alkenyl, or C₂-C₈ alkynyl, each of which is optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH; or R₄ is selected from the group consisting of C₁-C₈ alkylsulfonyl, formyl, hydroxy, C₁-C₈ alkoxyl, —SH, thio(C₂-C₈)alkyl, amino, C₁-C₈ alkylamino, and di(C₁-C₈ alkyl)amino;

R₅ represents 1-3 substituents, each of which is independently selected from the group consisting of C₁-C₈ alkyl, C₂-C₈ alkenyl, and C₂-C₈ alkynyl, each of which is optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, and di(C₁-C₈ alkyl)amino; or

R₅ represents 1-3 substituents, each of which is independently selected from the group consisting of halogen, C₁-C₈ alkylsulfonyl, formyl, COOH, hydroxy, C₁-C₈ alkoxyl, —SH, thio(C₂-C₈)alkyl, amino, C₁-C₈ alkylamino, and di(C₁-C₈ alkyl)amino;

R₆ is OP(O)(OH)₂, OH, OC(O)R₇, OSO₂OH, SO₂NH₂ or OR₇; and

R₇ is C₁-C₈ alkyl, optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH; phenyl, aralkyl or benzyl;

provided that if R₅ is hydroxy, then R₆ is not hydroxy or alkoxy;

or a pharmaceutically acceptable salt thereof.

In some embodiments, A is C₁-C₄ alkylene, e.g., C₂ alkylene.

In some embodiments, R₁ is hydrogen.

In some embodiments, R₁ is C₁-C₈ alkyl, e.g., C₁ alkyl or C₂ alkyl. In some embodiments, R₁ is C₁-C₈ alkyl substituted with 1-3 substituents. In some embodiments, R₁ is C₁-C₈ alkyl substituted with 1-3 substituents, wherein at least one substituent is halogen (e.g., fluorine). In some embodiments, C₂ alkyl substituted with 1-3 substituents, e.g., 1-3 halogens, e.g., 1-3 fluorines. In some embodiments, R₁ is C₂ alkyl substituted with 3 fluorines. In some embodiments, R₁ is —CH₂—CF₃.

In some embodiments, R¹ is C₁-C₈ alkyl, C₂-C₈ alkenyl, or C₂-C₈ alkynyl, wherein each carbon of the C₁-C₈ alkyl, C₂-C₈ alkenyl, or C₂-C₈ alkynyl is substituted with no more than 2 substituents, each of which is independently selected from the group consisting of cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH.

In some embodiments, R₂ is hydrogen. In some embodiments, R₂ is C₁-C₈ alkyl, e.g., C₁ alkyl or C₂ alkyl. In some embodiments, R₂ is C₁-C₈ alkyl substituted with 1-3 substituents. In some embodiments, R₂ is C₁-C₈ alkyl substituted with 1-3 substituents, wherein at least one substituent is halogen (e.g., fluorine). In some embodiments, R₂ is C₂ alkyl substituted with 1-3 substituents, e.g., 1-3 halogens, e.g., 1-3 fluorines. In some embodiments, C₂ alkyl substituted with 3 fluorines. In some embodiments, R₂ is —CH₂—CF₃.

In some embodiments, R₂ is C₁-C₈ alkyl, C₂-C₈ alkenyl, or C₂-C₈ alkynyl, wherein each carbon of the C₁-C₈ alkyl, C₂-C₈ alkenyl, or C₂-C₈ alkynyl is substituted with no more than 2 substituents, each of which is independently selected from the group consisting of cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH.

In some embodiments, R₁ and R₂ are both C₁-C₈ alkyl. In some embodiments, R₁ and R₂ are both C₁ alkyl. In some embodiments, R₁ and R₂ are both C₂ alkyl. In some embodiments, R₁ is C₁ alkyl and R₂ is C₂ alkyl. In some embodiments, R₁ is C₂ alkyl and R₂ is C₁ alkyl.

In some embodiments, both R₁ and R₂ are C₁-C₈ alkyl substituted with 1-3 substituents. In some embodiments, both R₁ and R₂ are C₁-C₈ alkyl substituted with 1-3 substituents, wherein at least one substituent is halogen (e.g., fluorine). In some embodiments, both R₁ and R₂ are C₂ alkyl substituted with 1-3 substituents (e.g., 1-3 halogens, e.g., 1-3 fluorines). In some embodiments, both R₁ and R₂ are C₂ alkyl substituted with 3 fluorines. In some embodiments, both R₁ and R₂ are —CH₂—CF₃.

In some embodiments, R₁ is hydrogen and R₂ is C₁-C₈ alkyl (e.g., C₁ alkyl or C₂ alkyl).

In some embodiments, R₁ and R₂, together with the nitrogen to which they are attached, form a group selected from the following:

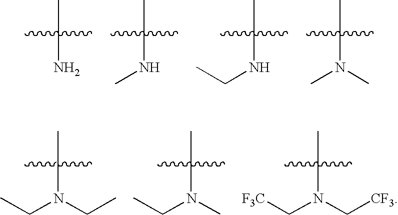

In some embodiments, R₃ is hydrogen.

In some embodiments, R³ is C₁-C₈ alkyl, C₂-C₈ alkenyl, or C₂-C₈ alkynyl, wherein each carbon of the C₁-C₈ alkyl, C₂-C₈ alkenyl, or C₂-C₈ alkynyl is substituted with no more than 2 substituents, each of which is independently selected from the group consisting of cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH.

In some embodiments, R₄ is C₁-C₈ alkyl, e.g., C₁, C₂, C₃ or C₄ alkyl.

In some embodiments, R⁴ is C₁-C₈ alkyl, C₂-C₈ alkenyl, or C₂-C₈ alkynyl, wherein each carbon of the C₁-C₈ alkyl, C₂-C₈ alkenyl, or C₂-C₈ alkynyl is substituted with no more than 2 substituents, each of which is independently selected from the group consisting of cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH.

In some embodiments, R₅ represents 1 substituent. In some embodiments, R₅ is halogen, e.g., fluorine.

In some embodiments, R₅ represents 2 substituents. In some embodiments, both R₅ substituents are halogen, e.g., fluorine.

In some embodiments, R⁵ represents 1-3 substituents, each of which is independently selected from the group consisting of C₁-C₈ alkyl, C₂-C₈ alkenyl, or C₂-C₈ alkynyl, wherein each carbon of the C₁-C₈ alkyl, C₂-C₈ alkenyl, or C₂-C₈ alkynyl is substituted with no more than 2 substituents, each of which is independently selected from the group consisting of cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH.

In some embodiments, R₆ is OH. In some embodiments, R₆ is OR₇. In some embodiments, R₇ is C₁-C₈ alkyl, e.g., C₁ alkyl. In some embodiments, R₇ is C₁ alkyl substituted with 1-3 substituents. In some embodiments, R₇ is C₁ alkyl substituted with 1-3 halogens, e.g., 1-3 fluorines. In some embodiments, R₇ is C₁ alkyl substituted with 2 fluorines. In some embodiments, R₇ is C₁ alkyl substituted with 3 fluorines.

In some embodiments, R₇ is C₂ alkyl. In some embodiments, R₇ is C₂ alkyl substituted with 1-3 substituents. In some embodiments, R₇ is C₂ alkyl substituted with 1 substituent. In some embodiments, R₇ is —CH₂—CH₂—OH. In some embodiments, R₇ is —CH₂—CH₂—(C₁-C₈ alkoxyl) (e.g., R₇ is —CH₂—CH₂—O—CH₃). In some embodiments, R₇ is —CH₂—CH₂— (di(C₁-C₈ alkyl)amino) (e.g., R₇ is —CH₂—CH₂—N(CH₃)₂).

In some embodiments, R₇ is C₃ alkyl.

In some embodiments, R⁷ is C₁-C₈ alkyl, wherein each carbon of the C₁-C₈ alkyl is substituted with no more than 2 substituents, each of which is independently selected from the group consisting of cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH.

In one aspect, the invention features a pharmaceutical composition comprising a compound of formula II.

In one aspect, the invention features a dosage form comprising a compound of formula II. In some embodiments, the dosage form is an oral dosage form.

In one aspect, the invention features a method for the treatment of obesity in a subject, the method comprising administering to the subject a compound of formula II, such that obesity is treated.

In one aspect, the invention features a method for the treatment of Obsessive Compulsive Disorder (OCD) in a subject, the method comprising administering to the subject a compound of formula II, such that OCD is treated.

In one aspect, the invention features a method for suppressing appetite in a subject, the method comprising administering to the subject a compound of formula II, such that appetite is suppressed in the subject.

In one aspect, the invention features a method for the treatment of schizophrenia or psychosis in a subject, the method comprising administering to the subject a compound of formula II, such that schizophrenia or psychosis is treated.

In one aspect, the invention features A method for the treatment of anxiety or depression in a subject, the method comprising administering to the subject a compound of formula II, such that anxiety or depression is treated in the subject.

In one aspect, the invention features A method for the treatment of diabetes in a subject, the method comprising administering to the subject a compound of formula II, such that diabetes is treated in the subject.

In one aspect, the invention features A method for the treatment of attention deficit hyperactivity disorder (ADHD) in a subject, the method comprising administering to the subject a compound of formula II, such that ADHD is treated in the subject.

In one aspect, the invention features A method for the treatment of suicidal behavior in a subject, the method comprising administering to the subject a compound of formula II, such that suicidal behavior is treated in the subject.

In one aspect, the invention features A method for the treatment of migraine in a subject, the method comprising administering to the subject a compound of formula II, such that migraine is treated in the subject.

In one aspect, the invention features a method for enhancing cognition in a subject, the method comprising administering to the subject a compound of formula II, such that cognition is enhanced in the subject.

In one aspect, the invention features a method for the treatment of a central nervous system disorder in a subject, the method comprising administering to the subject a compound of formula II, such that the central nervous system disorder is treated. In some embodiments, the central nervous system disorder is selected from the group consisting of epilepsy, Alzheimer's disease, sexual dysfunction, addiction, anorexia nervosa, Tourette's syndrome, and trichotillomania.

In one aspect, the invention features a method for the treatment of acral lick dermatitis (ALD) in a canine subject, the method comprising administering to the subject a compound of formula II, such that acral lick dermatitis is treated.

In one aspect, the invention features a method of increasing the activity of a serotonin receptor, the method comprising contacting a serotonin receptor with a compound of formula II. In some embodiments, the serotonin receptor is a 5-HT_{2C} receptor.

In one aspect, the invention features a compound represented by the structural formula III:

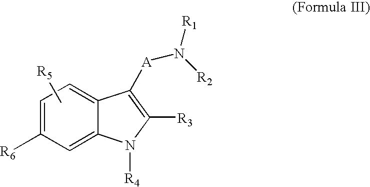

in which

A is C₁-C₄ alkylene, C₂-C₄ alkenylene, or C₂-C₄ alkynylene;

R₁ and R₂ are, independently for each occurrence, H, C₁-C₈ alkyl, C₂-C₈ alkenyl, or C₂-C₈ alkynyl, wherein the C₁-C₈ alkyl, C₂-C₈ alkenyl, or C₂-C₈ alkynyl are optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH;

R₃ is H, C₁-C₈ alkyl, C₂-C₈ alkenyl, or C₂-C₈ alkynyl, wherein the C₁-C₈ alkyl, C₂-C₈ alkenyl, or C₂-C₈ alkynyl are optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH; or R₃ is selected from the group consisting of halogen, C₁-C₈ alkylsulfonyl, formyl, COOH, hydroxy, C₁-C₈ alkoxyl, —SH, thio(C₂-C₈)alkyl, amino, C₁-C₈ alkylamino, and di(C₁-C₈ alkyl)amino;

R₄ is C₁-C₈ alkyl, C₂-C₈ alkenyl, or C₂-C₈ alkynyl, each of which is optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH; or R₄ is selected from the group consisting of C₁-C₈ alkylsulfonyl, formyl, hydroxy, C₁-C₈ alkoxyl, —SH, thio(C₂-C₈)alkyl, amino, C₁-C₈ alkylamino, and di(C₁-C₈ alkyl)amino;

R₅ represents 1-3 substituents, each of which is independently selected from the group consisting of C₁-C₈ alkyl, C₂-C₈ alkenyl, and C₂-C₈ alkynyl, each of which is optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, and di(C₁-C₈ alkyl)amino; or

R₅ represents 1-3 substituents, each of which is independently selected from the group consisting of halogen, C₁-C₈ alkylsulfonyl, formyl, COOH, hydroxy, C₁-C₈ alkoxyl, —SH, thio(C₂-C₈)alkyl, amino, C₁-C₈ alkylamino, and di(C₁-C₈ alkyl)amino;

R₆ is OP(O)(OH)₂, OH, OC(O)R₇, OSO₂OH, SO₂NH₂ or OR₇; and

R₇ is C₁-C₈ alkyl, optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH; phenyl, aralkyl or benzyl;

or a pharmaceutically acceptable salt thereof.

In some embodiments, A is C₁-C₄ alkylene, e.g., C₂ alkylene.

In some embodiments, R₁ is hydrogen.

In some embodiments, R₁ is C₁-C₈ alkyl, e.g., C₁ alkyl or C₂ alkyl. In some embodiments, R₁ is C₁-C₈ alkyl substituted with 1-3 substituents. In some embodiments, R₁ is C₁-C₈ alkyl substituted with 1-3 substituents, wherein at least one substituent is halogen (e.g., fluorine). In some embodiments, R₁ is C₂ alkyl substituted with 1-3 substituents, e.g., 1-3 halogens, e.g., 1-3 fluorines. In some embodiments, R₁ is C₂ alkyl substituted with 3 fluorines. In some embodiments, R₁ is —CH₂—CF₃.

In some embodiments, R¹ is C₁-C₈ alkyl, C₂-C₈ alkenyl, or C₂-C₈ alkynyl, wherein each carbon of the C₁-C₈ alkyl, C₂-C₈ alkenyl, or C₂-C₈ alkynyl is substituted with no more than 2 substituents, each of which is independently selected from the group consisting of cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH.

In some embodiments, R₂ is hydrogen.

In some embodiments, R₂ is C₁-C₈ alkyl, e.g., C₁ alkyl or C₂ alkyl. In some embodiments, R₂ is C₁-C₈ alkyl substituted with 1-3 substituents. In some embodiments, R₂ is C₁-C₈ alkyl substituted with 1-3 substituents, wherein at least one substituent is halogen (e.g., fluorine). In some embodiments, R₂ is C₂ alkyl substituted with 1-3 substituents, e.g., 1-3 halogens, e.g., 1-3 fluorines. In some embodiments, R₂ is C₂ alkyl substituted with 3 fluorines. In some embodiments, R₂ is —CH₂—CF₃.

In some embodiments, R² is C₁-C₈ alkyl, C₂-C₈ alkenyl, or C₂-C₈ alkynyl, wherein each carbon of the C₁-C₈ alkyl, C₂-C₈ alkenyl, or C₂-C₈ alkynyl is substituted with no more than 2 substituents, each of which is independently selected from the group consisting of cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH.

In some embodiments, R₁ and R₂ are both C₁-C₈ alkyl, e.g., R₁ and R₂ are both C₁ alkyl or C₂ alkyl. In some embodiments, R₁ is C₁ alkyl and R₂ is C₂ alkyl. In some embodiments, R₁ is C₂ alkyl and R₂ is C₁ alkyl.

In some embodiments, both R₁ and R₂ are C₁-C₈ alkyl substituted with 1-3 substituents. In some embodiments, both R₁ and R₂ are C₁-C₈ alkyl substituted with 1-3 substituents, wherein at least one substituent is halogen (e.g., fluorine). In some embodiments, both R₁ and R₂ are C₂ alkyl substituted with 1-3 substituents, e.g., 1-3 halogens, e.g., 1-3 fluorines. In some embodiments, both R₁ and R₂ are C₂ alkyl substituted with 3 fluorines. In some embodiments, both R₁ and R₂ are —CH₂—CF₃.

In some embodiments, R₁ is hydrogen and R₂ is C₁-C₈ alkyl (e.g., C₁ alkyl or C₂ alkyl).

In some embodiments, R₁ and R₂, together with the nitrogen to which they are attached, form a group selected from the following:

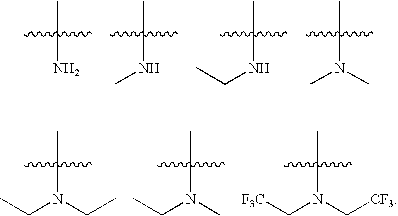

In some embodiments, R₃ is hydrogen.

In some embodiments, R³ is C₁-C₈ alkyl, C₂-C₈ alkenyl, or C₂-C₈ alkynyl, wherein each carbon of the C₁-C₈ alkyl, C₂-C₈ alkenyl, or C₂-C₈ alkynyl is substituted with no more than 2 substituents, each of which is independently selected from the group consisting of cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH.

In some embodiments, R₄ is C₁-C₈ alkyl, e.g., C₁, C₂, C₃ or C₄ alkyl.

In some embodiments, R⁴ is C₁-C₈ alkyl, C₂-C₈ alkenyl, or C₂-C₈ alkynyl, wherein each carbon of the C₁-C₈ alkyl, C₂-C₈ alkenyl, or C₂-C₈ alkynyl is substituted with no more than 2 substituents, each of which is independently selected from the group consisting of cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH.

In some embodiments, R₅ represents 1 substituent. In some embodiments, R₅ is halogen, e.g., fluorine. In some embodiments, R₅ represents 2 substituents. In some embodiments, both R₅ substituents are halogen, e.g., fluorine.

In some embodiments, R⁵ represents 1-3 substituents, each of which is independently selected from the group consisting of C₁-C₈ alkyl, C₂-C₈ alkenyl, or C₂-C₈ alkynyl, wherein each carbon of the C₁-C₈ alkyl, C₂-C₈ alkenyl, or C₂-C₈ alkynyl is substituted with no more than 2 substituents, each of which is independently selected from the group consisting of cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH.

In some embodiments, R₆ is OH. In some embodiments, R₆ is OR₇.

In some embodiments, R₇ is C₁-C₈ alkyl, e.g., C₁ alkyl. In some embodiments, R₇ is C₁ alkyl substituted with 1-3 substituents, e.g., 1-3 halogens, e.g., 1-3 fluorines. In some embodiments, R₇ is C₁ alkyl substituted with 2 fluorines. In some embodiments, R₇ is C₁ alkyl substituted with 3 fluorines.

In some embodiments, R₇ is C₂ alkyl. In some embodiments, R₇ is C₂ alkyl substituted with 1-3 substituents. In some embodiments, R₇ is C₂ alkyl substituted with 1 substituent. In some embodiments, R₇ is —CH₂—CH₂—OH. In some embodiments, R₇ is —CH₂—CH₂—(C₁-C₈ alkoxyl) (e.g., R₇ is —CH₂—CH₂—O—CH₃). In some embodiments, R₇ is —CH₂—CH₂— (di(C₁-C₈ alkyl)amino) (e.g., R₇ is —CH₂—CH₂—N(CH₃)₂).

In some embodiments, R₇ is C₃ alkyl.

In some embodiments, R⁷ is C₁-C₈ alkyl, wherein each carbon of the C₁-C₈ alkyl is substituted with no more than 2 substituents, each of which is independently selected from the group consisting of cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH.

In one aspect, the invention features a pharmaceutical composition comprising a compound of formula III.

In one aspect, the invention features a dosage form comprising a compound of formula III. In some embodiments, the dosage form is an oral dosage form.

In one aspect, the invention features a method for the treatment of obesity in a subject, the method comprising administering to the subject a compound of formula III, such that obesity is treated.

In one aspect, the invention features a method for the treatment of Obsessive Compulsive Disorder (OCD) in a subject, the method comprising administering to the subject a compound of formula III, such that OCD is treated.

In one aspect, the invention features a method for suppressing appetite in a subject, the method comprising administering to the subject a compound of formula III, such that appetite is suppressed in the subject.

In one aspect, the invention features a method for the treatment of schizophrenia or psychosis in a subject, the method comprising administering to the subject a compound of formula III, such that schizophrenia or psychosis is treated.

In one aspect, the invention features A method for the treatment of anxiety or depression in a subject, the method comprising administering to the subject a compound of formula III, such that anxiety or depression is treated in the subject.

In one aspect, the invention features A method for the treatment of diabetes in a subject, the method comprising administering to the subject a compound of formula III, such that diabetes is treated in the subject.

In one aspect, the invention features A method for the treatment of attention deficit hyperactivity disorder (ADHD) in a subject, the method comprising administering to the subject a compound of formula III, such that ADHD is treated in the subject.

In one aspect, the invention features A method for the treatment of suicidal behavior in a subject, the method comprising administering to the subject a compound of formula III, such that suicidal behavior is treated in the subject.

In one aspect, the invention features A method for the treatment of migraine in a subject, the method comprising administering to the subject a compound of formula III, such that migraine is treated in the subject.

In one aspect, the invention features a method for enhancing cognition in a subject, the method comprising administering to the subject a compound of formula III, such that cognition is enhanced in the subject.

In one aspect, the invention features a method for the treatment of a central nervous system disorder in a subject, the method comprising administering to the subject a compound of formula III, such that the central nervous system disorder is treated. In some embodiments, the central nervous system disorder is selected from the group consisting of epilepsy, Alzheimer's disease, sexual dysfunction, addiction, anorexia nervosa, Tourette's syndrome, and trichotillomania.

In one aspect, the invention features a method for the treatment of acral lick dermatitis (ALD) in a canine subject, the method comprising administering to the subject a compound of formula III, such that acral lick dermatitis is treated.

In one aspect, the invention features a method of increasing the activity of a serotonin receptor, the method comprising contacting a serotonin receptor with a compound of formula III. In some embodiments, the serotonin receptor is a 5-HT_{2C} receptor.

In one aspect, the invention features a compound represented by the structural formula IV:

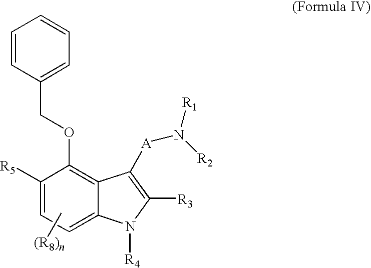

in which

A is C₁-C₄ alkylene, C₂-C₄ alkenylene, or C₂-C₄ alkynylene;

R₁ and R₂ are, independently for each occurrence, H, C₁-C₈ alkyl, C₂-C₈ alkenyl, or C₂-C₈ alkynyl, wherein the C₁-C₈ alkyl, C₂-C₈ alkenyl, or C₂-C₈ alkynyl are optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH;

R₃ is H, C₁-C₈ alkyl, C₂-C₈ alkenyl, or C₂-C₈ alkynyl, wherein the C₁-C₈ alkyl, C₂-C₈ alkenyl, or C₂-C₈ alkynyl are optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH; or R₃ is selected from the group consisting of halogen, C₁-C₈ alkylsulfonyl, formyl, COOH, hydroxy, C₁-C₈ alkoxyl, —SH, thio(C₂-C₈)alkyl, amino, C₁-C₈ alkylamino, and di(C₁-C₈ alkyl)amino;

R₄ is C₁-C₈ alkyl, C₂-C₈ alkenyl, or C₂-C₈ alkynyl, each of which is optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH; or R₄ is selected from the group consisting of C₁-C₈ alkylsulfonyl, formyl, hydroxy, C₁-C₈ alkoxyl, —SH, thio(C₂-C₈)alkyl, amino, C₁-C₈ alkylamino, and di(C₁-C₈ alkyl)amino;

R₅ is C₁-C₈ alkyl, C₂-C₈ alkenyl, or C₂-C₈ alkynyl, each of which is optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, and di(C₁-C₈ alkyl)amino; or

R₅ is halogen, C₁-C₈ alkylsulfonyl, formyl, COOH, hydroxy, C₁-C₈ alkoxyl, —SH, thio(C₂-C₈)alkyl, amino, C₁-C₈ alkylamino, or di(C₁-C₈ alkyl)amino;

R₈ is halogen, C₁-C₈ alkylsulfonyl, formyl, COOH, hydroxy, C₁-C₈ alkoxyl, —SH, thio(C₂-C₈)alkyl, amino, C₁-C₈ alkylamino, or di(C₁-C₈ alkyl)amino; and

n is 0, 1 or 2;

or a pharmaceutically acceptable salt thereof.

In some embodiments, A is C₁-C₄ alkylene, e.g., C₂ alkylene.

In some embodiments, R₁ is hydrogen.

In some embodiments, R₁ is C₁-C₈ alkyl, e.g., C₁ alkyl or C₂ alkyl. In some embodiments, R₁ is C₁-C₈ alkyl substituted with 1-3 substituents. In some embodiments, R₁ is C₁-C₈ alkyl substituted with 1-3 substituents, wherein at least one substituent is halogen (e.g., fluorine). In some embodiments, R₁ is C₂ alkyl substituted with 1-3 substituents, e.g., 1-3 halogens, e.g., 1-3 fluorines. In some embodiments, R₁ is C₂ alkyl substituted with 3 fluorines. In some embodiments, R₁ is —CH₂—CF₃.

In some embodiments, R¹ is C₁-C₈ alkyl, C₂-C₈ alkenyl, or C₂-C₈ alkynyl, wherein each carbon of the C₁-C₈ alkyl, C₂-C₈ alkenyl, or C₂-C₈ alkynyl is substituted with no more than 2 substituents, each of which is independently selected from the group consisting of cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH.

In some embodiments, R₂ is hydrogen.

In some embodiments, R₂ is C₁-C₈ alkyl, e.g., C₁ alkyl or C₂ alkyl. In some embodiments, R₂ is C₁-C₈ alkyl substituted with 1-3 substituents. In some embodiments, R₂ is C₁-C₈ alkyl substituted with 1-3 substituents, wherein at least one substituent is halogen (e.g., fluorine). In some embodiments, R₂ is C₂ alkyl substituted with 1-3 substituents, e.g., 1-3 halogens, e.g., 1-3 fluorines. In some embodiments, R₂ is C₂ alkyl substituted with 3 fluorines. In some embodiments, R₂ is —CH₂—CF₃.

In some embodiments, R² is C₁-C₈ alkyl, C₂-C₈ alkenyl, or C₂-C₈ alkynyl, wherein each carbon of the C₁-C₈ alkyl, C₂-C₈ alkenyl, or C₂-C₈ alkynyl is substituted with no more than 2 substituents, each of which is independently selected from the group consisting of cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH.

In some embodiments, R₁ and R₂ are both C₁-C₈ alkyl, e.g., C₁ alkyl or C₂ alkyl. In some embodiments, R₁ is C₁ alkyl and R₂ is C₂ alkyl. In some embodiments, both R₁ and R₂ are C₁-C₈ alkyl substituted with 1-3 substituents. In some embodiments, both R₁ and R₂ are C₁-C₈ alkyl substituted with 1-3 substituents, wherein at least one substituent is halogen (e.g., fluorine). In some embodiments, both R₁ and R₂ are C₂ alkyl substituted with 1-3 substituents, e.g., 1-3 halogens, e.g., 1-3 fluorines. In some embodiments, both R₁ and R₂ are C₂ alkyl substituted with 3 fluorines. In some embodiments, both R₁ and R₂ are —CH₂—CF₃.

In some embodiments, R₁ is hydrogen and R₂ is C₁-C₈ alkyl (e.g., C₁ alkyl or C₂ alkyl).

In some embodiments, R₁ and R₂, together with the nitrogen to which they are attached, form a group selected from the following:

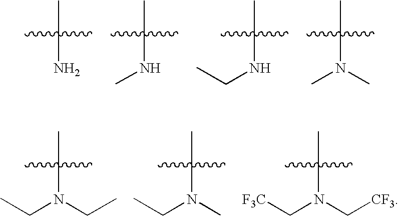

In some embodiments, R₃ is hydrogen.

In some embodiments, R³ is C₁-C₈ alkyl, C₂-C₈ alkenyl, or C₂-C₈ alkynyl, wherein each carbon of the C₁-C₈ alkyl, C₂-C₈ alkenyl, or C₂-C₈ alkynyl is substituted with no more than 2 substituents, each of which is independently selected from the group consisting of cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH.

In some embodiments, R₄ is C₁-C₈ alkyl, e.g., C₁, C₂, C₃ or C₄ alkyl.

In some embodiments, R⁴ is C₁-C₈ alkyl, C₂-C₈ alkenyl, or C₂-C₈ alkynyl, wherein each carbon of the C₁-C₈ alkyl, C₂-C₈ alkenyl, or C₂-C₈ alkynyl is substituted with no more than 2 substituents, each of which is independently selected from the group consisting of cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH.

In some embodiments, R₅ is halogen, e.g., fluorine.

In some embodiments, R⁵ is C₁-C₈ alkyl, C₂-C₈ alkenyl, or C₂-C₈ alkynyl, wherein each carbon is substituted with no more than 2 substituents, each of which is independently selected from the group consisting of cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH.

In some embodiments, n is 0.

In some embodiments, n is 1. In some embodiments, R₈ is halogen, e.g., fluorine.

In one aspect, the invention features a pharmaceutical composition comprising a compound of formula IV.

In one aspect, the invention features a dosage form comprising a compound of formula IV. In some embodiments, the dosage form is an oral dosage form.

In one aspect, the invention features a method for the treatment of obesity in a subject, the method comprising administering to the subject a compound of formula IV, such that obesity is treated.

In one aspect, the invention features a method for the treatment of Obsessive Compulsive Disorder (OCD) in a subject, the method comprising administering to the subject a compound of formula IV, such that OCD is treated.

In one aspect, the invention features a method for suppressing appetite in a subject, the method comprising administering to the subject a compound of formula IV, such that appetite is suppressed in the subject.

In one aspect, the invention features a method for the treatment of schizophrenia or psychosis in a subject, the method comprising administering to the subject a compound of formula IV, such that schizophrenia or psychosis is treated.

In one aspect, the invention features A method for the treatment of anxiety or depression in a subject, the method comprising administering to the subject a compound of formula IV, such that anxiety or depression is treated in the subject.

In one aspect, the invention features A method for the treatment of diabetes in a subject, the method comprising administering to the subject a compound of formula IV, such that diabetes is treated in the subject.

In one aspect, the invention features A method for the treatment of attention deficit hyperactivity disorder (ADHD) in a subject, the method comprising administering to the subject a compound of formula IV, such that ADHD is treated in the subject.

In one aspect, the invention features A method for the treatment of suicidal behavior in a subject, the method comprising administering to the subject a compound of formula IV, such that suicidal behavior is treated in the subject.

In one aspect, the invention features A method for the treatment of migraine in a subject, the method comprising administering to the subject a compound of formula IV, such that migraine is treated in the subject.

In one aspect, the invention features a method for enhancing cognition in a subject, the method comprising administering to the subject a compound of formula IV, such that cognition is enhanced in the subject.

In one aspect, the invention features a method for the treatment of a central nervous system disorder in a subject, the method comprising administering to the subject a compound of formula IV, such that the central nervous system disorder is treated. In some embodiments, the central nervous system disorder is selected from the group consisting of epilepsy, Alzheimer's disease, sexual dysfunction, addiction, anorexia nervosa, Tourette's syndrome, and trichotillomania.

In one aspect, the invention features a method for the treatment of acral lick dermatitis (ALD) in a canine subject, the method comprising administering to the subject a compound of formula IV, such that acral lick dermatitis is treated.

In one aspect, the invention features a method of increasing the activity of a serotonin receptor, the method comprising contacting a serotonin receptor with a compound of formula IV. In some embodiments, the serotonin receptor is a 5-HT_{2C} receptor.

In one aspect, the invention features a pharmaceutical composition comprising a compound of formula V:

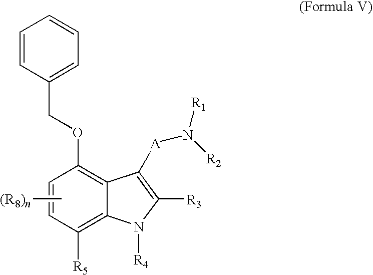

in which

A is C₁-C₄ alkylene, C₂-C₄ alkenylene, or C₂-C₄ alkynylene;

R₁ and R₂ are, independently for each occurrence, H, C₁-C₈ alkyl, C₂-C₈ alkenyl, or C₂-C₈ alkynyl, wherein the C₁-C₈ alkyl, C₂-C₈ alkenyl, or C₂-C₈ alkynyl are optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH;

R₃ is H, C₁-C₈ alkyl, C₂-C₈ alkenyl, or C₂-C₈ alkynyl, wherein the C₁-C₈ alkyl, C₂-C₈ alkenyl, or C₂-C₈ alkynyl are optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH; or R₃ is selected from the group consisting of halogen, C₁-C₈ alkylsulfonyl, formyl, COOH, hydroxy, C₁-C₈ alkoxyl, —SH, thio(C₂-C₈)alkyl, amino, C₁-C₈ alkylamino, and di(C₁-C₈ alkyl)amino;

R₄ is C₁-C₈ alkyl, C₂-C₈ alkenyl, or C₂-C₈ alkynyl, each of which is optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH; or R₄ is selected from the group consisting of C₁-C₈ alkylsulfonyl, formyl, hydroxy, C₁-C₈ alkoxyl, —SH, thio(C₂-C₈)alkyl, amino, C₁-C₈ alkylamino, and di(C₁-C₈ alkyl)amino;

R₅ is C₁-C₈ alkyl, C₂-C₈ alkenyl, or C₂-C₈ alkynyl, each of which is optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, and di(C₁-C₈ alkyl)amino; or

R₅ is halogen, C₁-C₈ alkylsulfonyl, formyl, COOH, hydroxy, C₁-C₈ alkoxyl, —SH, thio(C₂-C₈)alkyl, amino, C₁-C₈ alkylamino, or di(C₁-C₈ alkyl)amino;

R₈ is halogen, C₁-C₈ alkylsulfonyl, formyl, COOH, hydroxy, C₁-C₈ alkoxyl, —SH, thio(C₂-C₈)alkyl, amino, C₁-C₈ alkylamino, or di(C₁-C₈ alkyl)amino; and

n is 0, 1 or 2;

or a pharmaceutically acceptable salt thereof.

In some embodiments, A is C₁-C₄ alkylene, e.g., C₂ alkylene.

In some embodiments, R₁ is hydrogen.

In some embodiments, R₁ is C₁-C₈ alkyl, e.g., C₁ alkyl or C₂ alkyl. In some embodiments, R₁ is C₁-C₈ alkyl substituted with 1-3 substituents. In some embodiments, R₁ is C₁-C₈ alkyl substituted with 1-3 substituents, wherein at least one substituent is halogen (e.g., fluorine). In some embodiments, R₁ is C₂ alkyl substituted with 1-3 substituents, e.g., 1-3 halogens, e.g., 1-3 fluorines. In some embodiments, R₁ is C₂ alkyl substituted with 3 fluorines. In some embodiments, R₁ is —CH₂—CF₃.

In some embodiments, R¹ is C₁-C₈ alkyl, C₂-C₈ alkenyl, or C₂-C₈ alkynyl, wherein each carbon of the C₁-C₈ alkyl, C₂-C₈ alkenyl, or C₂-C₈ alkynyl is substituted with no more than 2 substituents, each of which is independently selected from the group consisting of cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH.

In some embodiments, R₂ is hydrogen.

In some embodiments, R₂ is C₁-C₈ alkyl, e.g., C₁ alkyl or C₂ alkyl. In some embodiments, R₂ is C₁-C₈ alkyl substituted with 1-3 substituents. In some embodiments, R₂ is C₁-C₈ alkyl substituted with 1-3 substituents, wherein at least one substituent is halogen (e.g., fluorine). In some embodiments, R₂ is C₂ alkyl substituted with 1-3 substituents, e.g., 1-3 halogens, e.g., 1-3 fluorines. In some embodiments, R₂ is C₂ alkyl substituted with 3 fluorines. In some embodiments, R₂ is —CH₂—CF₃.

In some embodiments, R² is C₁-C₈ alkyl, C₂-C₈ alkenyl, or C₂-C₈ alkynyl, wherein each carbon of the C₁-C₈ alkyl, C₂-C₈ alkenyl, or C₂-C₈ alkynyl is substituted with no more than 2 substituents, each of which is independently selected from the group consisting of cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH.

In some embodiments, R₁ and R₂ are both C₁-C₈ alkyl, e.g., C₁ alkyl or C₂ alkyl. In some embodiments, R₁ is C₁ alkyl and R₂ is C₂ alkyl. In some embodiments, both R₁ and R₂ are C₁-C₈ alkyl substituted with 1-3 substituents. In some embodiments, both R₁ and R₂ are C₁-C₈ alkyl substituted with 1-3 substituents, wherein at least one substituent is halogen (e.g., fluorine). In some embodiments, both R₁ and R₂ are C₂ alkyl substituted with 1-3 substituents, e.g., 1-3 halogens, e.g., 1-3 fluorines. In some embodiments, both R₁ and R₂ are C₂ alkyl substituted with 3 fluorines. In some embodiments, both R₁ and R₂ are —CH₂—CF₃.

In some embodiments, R₁ is hydrogen and R₂ is C₁-C₈ alkyl (e.g., C₁ alkyl or C₂ alkyl).

In some embodiments, R₁ and R₂, together with the nitrogen to which they are attached, form a group selected from the following:

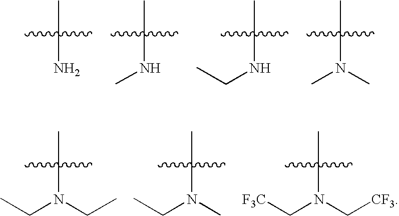

In some embodiments, R₃ is hydrogen.

In some embodiments, R³ is C₁-C₈ alkyl, C₂-C₈ alkenyl, or C₂-C₈ alkynyl, wherein each carbon of the C₁-C₈ alkyl, C₂-C₈ alkenyl, or C₂-C₈ alkynyl is substituted with no more than 2 substituents, each of which is independently selected from the group consisting of cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH.

In some embodiments, R₄ is C₁-C₈ alkyl, e.g., C₁, C₂, C₃ or C₄ alkyl.

In some embodiments, R⁴ is C₁-C₈ alkyl, C₂-C₈ alkenyl, or C₂-C₈ alkynyl, wherein each carbon of the C₁-C₈ alkyl, C₂-C₈ alkenyl, or C₂-C₈ alkynyl is substituted with no more than 2 substituents, each of which is independently selected from the group consisting of cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH.

In some embodiments, R₅ is halogen, e.g., fluorine.

In some embodiments, R⁵ is C₁-C₈ alkyl, C₂-C₈ alkenyl, or C₂-C₈ alkynyl, wherein each carbon of the C₁-C₈ alkyl, C₂-C₈ alkenyl, or C₂-C₈ alkynyl is substituted with no more than 2 substituents, each of which is independently selected from the group consisting of cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH.

In some embodiments, n is 0. In some embodiments, n is 1.

In some embodiments, R₈ is halogen, e.g., fluorine.

In one aspect, the invention features a dosage form comprising a compound of formula V. In some embodiments, the dosage form is an oral dosage form.

In one aspect, the invention features a method for the treatment of obesity in a subject, the method comprising administering to the subject a compound of formula V, such that obesity is treated.

In one aspect, the invention features a method for the treatment of Obsessive Compulsive Disorder (OCD) in a subject, the method comprising administering to the subject a compound of formula V, such that OCD is treated.

In one aspect, the invention features a method for suppressing appetite in a subject, the method comprising administering to the subject a compound of formula V, such that appetite is suppressed in the subject.

In one aspect, the invention features a method for the treatment of schizophrenia or psychosis in a subject, the method comprising administering to the subject a compound of formula V, such that schizophrenia or psychosis is treated.

In one aspect, the invention features A method for the treatment of anxiety or depression in a subject, the method comprising administering to the subject a compound of formula V, such that anxiety or depression is treated in the subject.

In one aspect, the invention features A method for the treatment of diabetes in a subject, the method comprising administering to the subject a compound of formula V, such that diabetes is treated in the subject.

In one aspect, the invention features A method for the treatment of attention deficit hyperactivity disorder (ADHD) in a subject, the method comprising administering to the subject a compound of formula V, such that ADHD is treated in the subject.

In one aspect, the invention features A method for the treatment of suicidal behavior in a subject, the method comprising administering to the subject a compound of formula V, such that suicidal behavior is treated in the subject.

In one aspect, the invention features A method for the treatment of migraine in a subject, the method comprising administering to the subject a compound of formula V, such that migraine is treated in the subject.

In one aspect, the invention features a method for enhancing cognition in a subject, the method comprising administering to the subject a compound of formula V, such that cognition is enhanced in the subject.

In one aspect, the invention features a method for the treatment of a central nervous system disorder in a subject, the method comprising administering to the subject a compound of formula V, such that the central nervous system disorder is treated. In some embodiments, the central nervous system disorder is selected from the group consisting of epilepsy, Alzheimer's disease, sexual dysfunction, addiction, anorexia nervosa, Tourette's syndrome, and trichotillomania.

In one aspect, the invention features a method for the treatment of acral lick dermatitis (ALD) in a canine subject, the method comprising administering to the subject a compound of formula V, such that acral lick dermatitis is treated.

In one aspect, the invention features a method of increasing the activity of a serotonin receptor, the method comprising contacting a serotonin receptor with a compound of formula V. In some embodiments, the serotonin receptor is a 5-HT_{2C} receptor.

In one aspect, the invention features a pharmaceutical composition comprising a compound of formula VI:

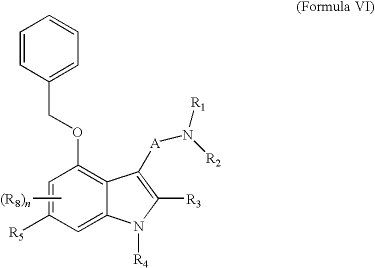

in which

A is C₁-C₄ alkylene, C₂-C₄ alkenylene, or C₂-C₄ alkynylene;

R₁ and R₂ are, independently for each occurrence, H, C₁-C₈ alkyl, C₂-C₈ alkenyl, or C₂-C₈ alkynyl, wherein the C₁-C₈ alkyl, C₂-C₈ alkenyl, or C₂-C₈ alkynyl are optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH;

R₃ is hydrogen, C₁-C₈ alkyl, C₂-C₈ alkenyl, or C₂-C₈ alkynyl, wherein the C₁-C₈ alkyl, C₂-C₈ alkenyl, or C₂-C₈ alkynyl are optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH; or R₃ is selected from the group consisting of halogen, C₁-C₈ alkylsulfonyl, formyl, COOH, hydroxy, C₁-C₈ alkoxyl, —SH, thio(C₂-C₈)alkyl, amino, C₁-C₈ alkylamino, and di(C₁-C₈ alkyl)amino;

R₄ is C₁-C₈ alkyl, C₂-C₈ alkenyl, or C₂-C₈ alkynyl, each of which is optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH; or R₄ is selected from the group consisting of C₁-C₈ alkylsulfonyl, formyl, hydroxy, C₁-C₈ alkoxyl, —SH, thio(C₂-C₈)alkyl, amino, C₁-C₈ alkylamino, and di(C₁-C₈ alkyl)amino;

R₅ is C₁-C₈ alkyl, C₂-C₈ alkenyl, or C₂-C₈ alkynyl, each of which is optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, and di(C₁-C₈ alkyl)amino; or

R₅ is halogen, C₁-C₈ alkylsulfonyl, formyl, COOH, hydroxy, C₁-C₈ alkoxyl, —SH, thio(C₂-C₈)alkyl, amino, C₁-C₈ alkylamino, or di(C₁-C₈ alkyl)amino;

R₈ is halogen, C₁-C₈ alkylsulfonyl, formyl, COOH, hydroxy, C₁-C₈ alkoxyl, —SH, thio(C₂-C₈)alkyl, amino, C₁-C₈ alkylamino, or di(C₁-C₈ alkyl)amino; and

n is 0, 1 or 2;

or a pharmaceutically acceptable salt thereof.

In some embodiments, A is C₁-C₄ alkylene, e.g., C₂ alkylene.

In some embodiments, R₁ is hydrogen.

In some embodiments, R₁ is C₁-C₈ alkyl, e.g., C₁ alkyl or C₂ alkyl. In some embodiments, R₁ is C₁-C₈ alkyl substituted with 1-3 substituents. In some embodiments, R₁ is C₁-C₈ alkyl substituted with 1-3 substituents, wherein at least one substituent is halogen (e.g., fluorine). In some embodiments, R₁ is C₂ alkyl substituted with 1-3 substituents, e.g., 1-3 halogens, e.g., 1-3 fluorines. In some embodiments, R₁ is C₂ alkyl substituted with 3 fluorines. In some embodiments, R₁ is —CH₂—CF₃.

In some embodiments, R¹ is C₁-C₈ alkyl, C₂-C₈ alkenyl, or C₂-C₈ alkynyl, wherein each carbon of the C₁-C₈ alkyl, C₂-C₈ alkenyl, or C₂-C₈ alkynyl is substituted with no more than 2 substituents, each of which is independently selected from the group consisting of cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH.

In some embodiments, R₂ is hydrogen.

In some embodiments, R₂ is C₁-C₈ alkyl, e.g., C₁ alkyl or C₂ alkyl. In some embodiments, R₂ is C₁-C₈ alkyl substituted with 1-3 substituents. In some embodiments, R₂ is C₁-C₈ alkyl substituted with 1-3 substituents, wherein at least one substituent is halogen (e.g., fluorine). In some embodiments, R₂ is C₂ alkyl substituted with 1-3 substituents, e.g., 1-3 halogens, e.g., 1-3 fluorines. In some embodiments, R₂ is C₂ alkyl substituted with 3 fluorines. In some embodiments, R₂ is —CH₂—CF₃.

In some embodiments, R₂ is C₁-C₈ alkyl, C₂-C₈ alkenyl, or C₂-C₈ alkynyl, wherein each carbon of the C₁-C₈ alkyl, C₂-C₈ alkenyl, or C₂-C₈ alkynyl is substituted with no more than 2 substituents, each of which is independently selected from the group consisting of cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH.

In some embodiments, R₁ and R₂ are both C₁-C₈ alkyl, e.g., C₁ alkyl or C₂ alkyl. In some embodiments, R₁ is C₁ alkyl and R₂ is C₂ alkyl. In some embodiments, both R₁ and R₂ are C₁-C₈ alkyl substituted with 1-3 substituents. In some embodiments, both R₁ and R₂ are C₁-C₈ alkyl substituted with 1-3 substituents, wherein at least one substituent is halogen (e.g., fluorine).

In some embodiments, both R₁ and R₂ are C₂ alkyl substituted with 1-3 substituents. In some embodiments, both R₁ and R₂ are C₂ alkyl substituted with 1-3 halogens, e.g., 1-3 fluorines. In some embodiments, both R₁ and R₂ are C₂ alkyl substituted with 3 fluorines. In some embodiments, both R₁ and R₂ are —CH₂—CF₃.

In some embodiments, R₁ is hydrogen and R₂ is C₁-C₈ alkyl (e.g., C₁ alkyl or C₂ alkyl).

In some embodiments, R₁ and R₂, together with the nitrogen to which they are attached, form a group selected from the following:

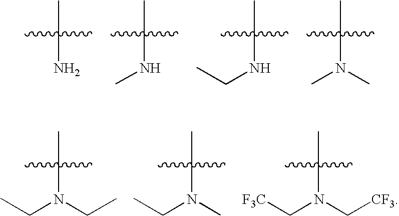

In some embodiments, R₃ is hydrogen.

In some embodiments, R³ is C₁-C₈ alkyl, C₂-C₈ alkenyl, or C₂-C₈ alkynyl, wherein each carbon of the C₁-C₈ alkyl, C₂-C₈ alkenyl, or C₂-C₈ alkynyl is substituted with no more than 2 substituents, each of which is independently selected from the group consisting of cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH.

In some embodiments, R₄ is C₁-C₈ alkyl, e.g., C₁, C₂, C₃ or C₄ alkyl.

In some embodiments, R⁴ is C₁-C₈ alkyl, C₂-C₈ alkenyl, or C₂-C₈ alkynyl, wherein each carbon of the C₁-C₈ alkyl, C₂-C₈ alkenyl, or C₂-C₈ alkynyl is substituted with no more than 2 substituents, each of which is independently selected from the group consisting of cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH.

In some embodiments, R₅ is halogen, e.g., fluorine.

In some embodiments, R₅ is C₁-C₈ alkyl, C₂-C₈ alkenyl, or C₂-C₈ alkynyl, wherein each carbon of the C₁-C₈ alkyl, C₂-C₈ alkenyl, or C₂-C₈ alkynyl is substituted with no more than 2 substituents, each of which is independently selected from the group consisting of cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH.

In some embodiments, n is 0. In some embodiments, n is 1.

In some embodiments, R₈ is halogen, e.g., fluorine.

In one aspect, the invention features a dosage form comprising a compound of formula VI. In some embodiments, the dosage form is an oral dosage form.

In one aspect, the invention features a method for the treatment of obesity in a subject, the method comprising administering to the subject a compound of formula VI, such that obesity is treated.

In one aspect, the invention features a method for the treatment of Obsessive Compulsive Disorder (OCD) in a subject, the method comprising administering to the subject a compound of formula VI, such that OCD is treated.

In one aspect, the invention features a method for suppressing appetite in a subject, the method comprising administering to the subject a compound of formula VI, such that appetite is suppressed in the subject.

In one aspect, the invention features a method for the treatment of schizophrenia or psychosis in a subject, the method comprising administering to the subject a compound of formula VI, such that schizophrenia or psychosis is treated.

In one aspect, the invention features A method for the treatment of anxiety or depression in a subject, the method comprising administering to the subject a compound of formula VI, such that anxiety or depression is treated in the subject.

In one aspect, the invention features A method for the treatment of diabetes in a subject, the method comprising administering to the subject a compound of formula VI, such that diabetes is treated in the subject.

In one aspect, the invention features A method for the treatment of attention deficit hyperactivity disorder (ADHD) in a subject, the method comprising administering to the subject a compound of formula VI, such that ADHD is treated in the subject.

In one aspect, the invention features A method for the treatment of suicidal behavior in a subject, the method comprising administering to the subject a compound of formula VI, such that suicidal behavior is treated in the subject.

In one aspect, the invention features A method for the treatment of migraine in a subject, the method comprising administering to the subject a compound of formula VI, such that migraine is treated in the subject.

In one aspect, the invention features a method for enhancing cognition in a subject, the method comprising administering to the subject a compound of formula VI, such that cognition is enhanced in the subject.

In one aspect, the invention features a method for the treatment of a central nervous system disorder in a subject, the method comprising administering to the subject a compound of formula VI, such that the central nervous system disorder is treated. In some embodiments, the central nervous system disorder is selected from the group consisting of epilepsy, Alzheimer's disease, sexual dysfunction, addiction, anorexia nervosa, Tourette's syndrome, and trichotillomania.

In one aspect, the invention features a method for the treatment of acral lick dermatitis (ALD) in a canine subject, the method comprising administering to the subject a compound of formula VI, such that acral lick dermatitis is treated.

In one aspect, the invention features a method of increasing the activity of a serotonin receptor, the method comprising contacting a serotonin receptor with a compound of formula VI. In some embodiments, the serotonin receptor is a 5-HT_{2C} receptor.

In one aspect, the invention features a compound represented by the structural formula VII:

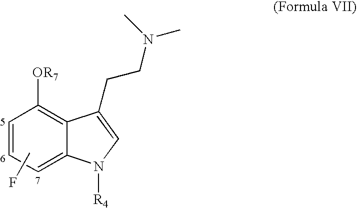

wherein:

F is in at least one of the 5, 6, or 7, positions; and

R₄ and R₇ are independently selected from C₁-C₈ alkyl;

or a pharmaceutically acceptable salt thereof.

In some embodiments, R₄ is C₁ alkyl. In some embodiments, R₄ is C₂ alkyl. In some embodiments, R₄ is C₃ alkyl. In some embodiments, R₇ is C₁ alkyl.

In some embodiments, R₇ is C₂ alkyl. In some embodiments, R₇ is C₃ alkyl.

In some embodiments, F is in the 5 position. In some embodiments, F is in the 6 position. In some embodiments, F is in the 7 position. In some embodiments, F is in the 5 and 6 positions. In some embodiments, F is in the 5 and 7 positions. In some embodiments, F is in the 6 and 7 positions.

In one aspect, the invention features a pharmaceutical composition comprising a compound of formula VII.

In one aspect, the invention features a dosage form comprising a compound of formula VII. In some embodiments, the dosage form is an oral dosage form.

In one aspect, the invention features a method for the treatment of obesity in a subject, the method comprising administering to the subject a compound of formula VII, such that obesity is treated.

In one aspect, the invention features a method for the treatment of Obsessive Compulsive Disorder (OCD) in a subject, the method comprising administering to the subject a compound of formula VII, such that OCD is treated.

In one aspect, the invention features a method for suppressing appetite in a subject, the method comprising administering to the subject a compound of formula VII, such that appetite is suppressed in the subject.

In one aspect, the invention features a method for the treatment of schizophrenia or psychosis in a subject, the method comprising administering to the subject a compound of formula VII, such that schizophrenia or psychosis is treated.

In one aspect, the invention features A method for the treatment of anxiety or depression in a subject, the method comprising administering to the subject a compound of formula VII, such that anxiety or depression is treated in the subject.

In one aspect, the invention features A method for the treatment of diabetes in a subject, the method comprising administering to the subject a compound of formula VII, such that diabetes is treated in the subject.

In one aspect, the invention features A method for the treatment of attention deficit hyperactivity disorder (ADHD) in a subject, the method comprising administering to the subject a compound of formula VII, such that ADHD is treated in the subject.

In one aspect, the invention features A method for the treatment of suicidal behavior in a subject, the method comprising administering to the subject a compound of formula VII, such that suicidal behavior is treated in the subject.

In one aspect, the invention features A method for the treatment of migraine in a subject, the method comprising administering to the subject a compound of formula VII, such that migraine is treated in the subject.

In one aspect, the invention features a method for enhancing cognition in a subject, the method comprising administering to the subject a compound of formula VII, such that cognition is enhanced in the subject.

In one aspect, the invention features a method for the treatment of a central nervous system disorder in a subject, the method comprising administering to the subject a compound of formula VII, such that the central nervous system disorder is treated. In some embodiments, the central nervous system disorder is selected from the group consisting of epilepsy, Alzheimer's disease, sexual dysfunction, addiction, anorexia nervosa, Tourette's syndrome, and trichotillomania.

In one aspect, the invention features a method for the treatment of acral lick dermatitis (ALD) in a canine subject, the method comprising administering to the subject a compound of formula VII, such that acral lick dermatitis is treated.

In one aspect, the invention features a method of increasing the activity of a serotonin receptor, the method comprising contacting a serotonin receptor with a compound of formula VII. In some embodiments, the serotonin receptor is a 5-HT_{2C} receptor.

In one aspect, the invention features a compound selected from the compounds disclosed in FIG. 10a or 10b, or a pharmaceutically acceptable salt thereof. In some embodiments, the compound is an HCl salt.

Other advantages, aspects, and embodiments of the invention will be apparent in light of the description herein.

### BRIEF DESCRIPTION OF THE DRAWINGS

FIG. 1 is a depiction of the structures of, and exemplary synthetic routes for preparation of, certain compounds of the invention.

FIG. 2 is a depiction of an exemplary synthetic route useful for preparation of certain compounds of the invention.

Figure is a depiction of the structures of, and exemplary synthetic routes for preparation of, certain compounds of the invention.

FIG. 4 is a depiction of the structures of, and an exemplary synthetic route for preparation of, certain compounds of the invention.

FIG. 5 is a depiction of the structures of, and exemplary synthetic routes for preparation of, certain compounds of the invention.

FIG. 6 is a depiction of the structures of, and an exemplary synthetic route for preparation of, certain compounds of the invention.

FIG. 7 is a depiction of the structures of, and an exemplary synthetic route for preparation of, certain compounds of the invention.

FIG. 8 is a depiction of the structure of a compound of the invention.

FIG. 9 is a depiction of the structures of, and an exemplary synthetic route for preparation of, certain compounds of the invention.

FIG. 10 is a depiction of certain compounds of the invention.

### DETAILED DESCRIPTION OF THE INVENTION

### 1. Definitions

Before further description of the present invention, and in order that the invention may be more readily understood, certain terms are first defined and collected here for convenience.

The term “administration” or “administering” includes routes of introducing the compound(s) of the invention to a subject to perform their intended function. Examples of routes of administration that may be used include injection (subcutaneous, intravenous, parenterally, intraperitoneally, intrathecal), oral, inhalation, rectal and transdermal. The pharmaceutical preparations may be given by forms suitable for each administration route. For example, these preparations are administered in tablets or capsule form, by injection, inhalation, eye lotion, ointment, suppository, etc. administration by injection, infusion or inhalation; topical by lotion or ointment; and rectal by suppositories. Oral administration is preferred. The injection can be bolus or can be continuous infusion. Depending on the route of administration, the compound of the invention can be coated with or disposed in a selected material to protect it from natural conditions which may detrimentally affect its ability to perform its intended function. The compound of the invention can be administered alone, or in conjunction with either another agent as described above or with a pharmaceutically-acceptable carrier, or both. The compound of the invention can be administered prior to the administration of the other agent, simultaneously with the agent, or after the administration of the agent. Furthermore, the compound of the invention can also be administered in a pro-drug form which is converted into its active metabolite, or more active metabolite in vivo.

The term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. The term alkyl further includes alkyl groups, which can further include oxygen, nitrogen, sulfur or phosphorous atoms replacing one or more carbons of the hydrocarbon backbone, e.g., oxygen, nitrogen, sulfur or phosphorous atoms. In preferred embodiments, a straight chain or branched chain alkyl has 30 or fewer carbon atoms in its backbone (e.g., C₁-C₃₀ for straight chain, C₃-C₃₀ for branched chain), preferably 26 or fewer, and more preferably 20 or fewer, and still more preferably 4 or fewer. Likewise, preferred cycloalkyls have from 3-10 carbon atoms in their ring structure, and more preferably have 3, 4, 5, 6 or 7 carbons in the ring structure.

Moreover, the term alkyl as used throughout the specification and sentences is intended to include both “unsubstituted alkyls” and “substituted alkyls,” the latter of which refers to alkyl moieties having substituents replacing a hydrogen on one or more carbons of the hydrocarbon backbone. Such substituents can include, for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkylaryl, or an aromatic or heteroaromatic moiety. It will be understood by those skilled in the art that the moieties substituted on the hydrocarbon chain can themselves be substituted, if appropriate. Cycloalkyls can be further substituted, e.g., with the substituents described above. An “alkylaryl” moiety is an alkyl substituted with an aryl (e.g., phenylmethyl (benzyl)). The term “alkyl” also includes unsaturated aliphatic groups analogous in length and possible substitution to the alkyls described above, but that contain at least one double or triple bond respectively.

Unless the number of carbons is otherwise specified, “lower alkyl” as used herein means an alkyl group, as defined above, but having from one to ten carbons, more preferably from one to six, and still more preferably from one to four carbon atoms in its backbone structure, which may be straight or branched-chain. Examples of lower alkyl groups include methyl, ethyl, n-propyl, i-propyl, tert-butyl, hexyl, heptyl, octyl and so forth. In preferred embodiment, the term “lower alkyl” includes a straight chain alkyl having 4 or fewer carbon atoms in its backbone, e.g., C₁-C₄ alkyl.

The terms “alkoxyalkyl,” “polyaminoalkyl” and “thioalkoxyalkyl” refer to alkyl groups, as described above, which further include oxygen, nitrogen or sulfur atoms replacing one or more carbons of the hydrocarbon backbone, e.g., oxygen, nitrogen or sulfur atoms.

The terms “alkenyl” and “alkynyl” refer to unsaturated aliphatic groups analogous in length and possible substitution to the alkyls described above, but that contain at least one double or triple bond, respectively. For example, the invention contemplates cyano and propargyl groups.

The terms “alkylene”, “alkenylene” and “alkynylene” refer to divalent aliphatic radicals corresponding respectively to alkyl, alkenyl, and alkynyl groups as defined above, and which may be substituted as described above.

The term “aryl” as used herein, refers to the radical of aryl groups, including 5- and 6-membered single-ring aromatic groups that may include from zero to four heteroatoms, for example, benzene, pyrrole, furan, thiophene, imidazole, benzoxazole, benzothiazole, triazole, tetrazole, pyrazole, pyridine, pyrazine, pyridazine and pyrimidine, and the like. Aryl groups also include polycyclic fused aromatic groups such as naphthyl, quinolyl, indolyl, and the like. Those aryl groups having heteroatoms in the ring structure may also be referred to as “aryl heterocycles,” “heteroaryls” or “heteroaromatics.” The aromatic ring can be substituted at one or more ring positions with such substituents as described above, as for example, halogen, hydroxyl, alkoxy, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings which are not aromatic so as to form a polycycle (e.g., tetralin).

The language “biological activities” of a compound of the invention includes all activities elicited by compound of the inventions in a responsive subject or cell. It includes genomic and non-genomic activities elicited by these compounds.

The term “chiral” refers to molecules which have the property of non-superimposability of the mirror image partner, while the term “achiral” refers to molecules which are superimposable on their mirror image partner.

The term “diastereomers” refers to stereoisomers with two or more centers of dissymmetry and whose molecules are not mirror images of one another.

The term “effective amount” includes an amount effective, at dosages and for periods of time necessary, to achieve the desired result, e.g., suppress appetite in a subject and/or treat a disorder described herein such as a serotonin receptor related disorder. Exemplary disorders include obesity; a disorder wherein appetite suppression is desirable; a disorder in which treating weight gain is desirable; a disorder in which cognitive enhancement is desirable; depressive disorders (e.g., depression, atypical depression, major depressive disorder, dysthymic disorder, and substance-induced mood disorder); bipolar disorders (e.g., bipolar I disorder, bipolar II disorder, and cyclothymic disorder); anxiety disorders (e.g., panic attack, agoraphobia, panic disorder, specific phobia, social phobia, obsessive compulsive disorder, posttraumatic stress disorder, acute stress disorder, generalized anxiety disorder, separation anxiety disorder, and substance-induced anxiety disorder); mood episodes (e.g., major depressive episode, manic episode, mixed episode, and hypomanic episode); adjustment disorders (e.g., adjustment disorder with anxiety and/or depressed mood); intellectual deficit disorders (e.g., dementia, Alzheimer's disease, and memory deficit); eating disorders (e.g., hyperphagia, bulimia or anorexia nervosa); schizophrenia (e.g., paranoid type, disorganized type, catatonic type, and undifferentiated type); schizophreniform disorder, schizoaffective disorder, delusional disorder, other psychotic disorders (e.g., substance-induced psychotic disorder, L-DOPA-induced psychosis, psychosis associated with Alzheimer's dementia, psychosis associated with Parkinson's disease, psychosis associated with Lewy body disease); sleep disorders (e.g., sleep apnea); suicidal behaviors; sexual dysfunction; migraine; cephalic pain or other pain; raised intracranial pressure; epilepsy; personality disorders; age-related behavioral disorders; behavioral disorders associated with dementia; organic mental disorders; mental disorders in childhood; aggressivity; age-related memory disorders; chronic fatigue syndrome; addiction (e.g., drug and alcohol addiction); premenstrual tension; damage of the central nervous system such as by trauma, stroke, neurodegenerative diseases or toxic or infective CNS diseases such as encephalitis or meningitis; cardiovascular disorders (e.g., thrombosis); hypertension; hyperlipidemia; arterial constriction; osteoarthritis; gall bladder disease; gout; gastrointestinal disorders (e.g., dysfunction of gastrointestinal motility); diabetes mellitus (e.g., Type 2 diabetes mellitus) and diabetes insipidus; cancer; infertility; early mortality; spinal cord injuries; Tourette's syndrome; trichotillomania; other central nervous system disorders; attention deficit hyperactivity disorder (ADHD); canine veterinary diseases (e.g., acral lick dermatitis); and combinations of these disorders that may be present in a mammal.

An effective amount of compound of the invention may vary according to factors such as the disease state, age, and weight of the subject, and the ability of the compound of the invention to elicit a desired response in the subject. Dosage regimens may be adjusted to provide the optimum therapeutic response. An effective amount is also one in which any toxic or detrimental effects (e.g., side effects) of the compound of the invention are outweighed by the therapeutically beneficial effects.

A therapeutically effective amount of compound of the invention (i.e., an effective dosage) may range from about 0.001 to 50 mg/kg body weight, preferably about 0.01 to 40 mg/kg body weight, more preferably about 0.1 to 35 mg/kg body weight, still more preferably about 1 to 30 mg/kg, and even more preferably about 10 to 30 mg/kg. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a compound of the invention can include a single treatment or, preferably, can include a series of treatments. In one example, a subject is treated with a compound of the invention in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of a compound of the invention used for treatment may increase or decrease over the course of a particular treatment.

The term “enantiomers” refers to two stereoisomers of a compound which are non-superimposable mirror images of one another. An equimolar mixture of two enantiomers is called a “racemic mixture” or a “racemate.”

The term “haloalkyl” is intended to include alkyl groups as defined above that are mono-, di- or polysubstituted by halogen, e.g., fluoromethyl and trifluoromethyl.

The term “haloalkoxyl” is intended to include alkoxyl groups that are mono-, di- or polysubstituted by halogen, e.g., fluoromethoxyl or trifluoromethoxyl.

The term “halogen” designates —F, —Cl, —Br or —I.

The term “hydroxyl” means —OH.

The term “heteroatom” as used herein means an atom of any element other than carbon or hydrogen. Preferred heteroatoms are nitrogen, oxygen, sulfur and phosphorus.

The language “improved biological properties” refers to any activity inherent in a compound of the invention that enhances its effectiveness in vivo. In a preferred embodiment, this term refers to any qualitative or quantitative improved therapeutic property of a compound of the invention, such as reduced off-target effects.

The term “optionally substituted” is intended to encompass groups that are unsubstituted or are substituted by other than hydrogen at one or more available positions, typically 1, 2, 3, 4 or 5 positions, by one or more suitable groups (which may be the same or different). Such optional substituents include, for example, hydroxy, halogen, cyano, nitro, C₁-C₈alkyl, C₂-C₈ alkenyl, C₂-C₈alkynyl, C₁-C₈alkoxy, C₂-C₈alkyl ether, C₃-C₈alkanone, C₁-C₈alkylthio, amino, mono- or di-(C₁-C₈alkyl)amino, haloC₁-C₈alkyl, haloC₁-C₈alkoxy, C₁-C₈alkanoyl, C₂-C₈alkanoyloxy, C₁-C₈alkoxycarbonyl, —COOH, —CONH₂, mono- or di-(C₁-C₈alkyl)aminocarbonyl, —SO₂NH₂, and/or mono or di(C₁-C₈alkyl)sulfonamido, as well as carbocyclic and heterocyclic groups. Optional substitution is also indicated by the phrase “substituted with from 0 to X substituents,” where X is the maximum number of possible substituents. Certain optionally substituted groups are substituted with from 0 to 2, 3 or 4 independently selected substituents (i.e., are unsubstituted or substituted with up to the recited maximum number of substituents).

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc.

The term “isomers” or “stereoisomers” refers to compounds which have identical chemical constitution, but differ with regard to the arrangement of the atoms or groups in space.

The term “modulate” refers to an increase or decrease, e.g., in the activity of a serotonin receptor in response to exposure to a compound of the invention, e.g., the stimulation of serotonin receptor activity of at least a sub-population of cells in an animal such that a desired end result is achieved, e.g., a therapeutic result.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The terms “polycyclyl” or “polycyclic radical” refer to the radical of two or more cyclic rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkyl, alkylaryl, or an aromatic or heteroaromatic moiety.

The term “prodrug” or “pro-drug” includes compounds with moieties that can be metabolized in vivo. Generally, the prodrugs are metabolized in vivo by esterases or by other mechanisms to active drugs. Examples of prodrugs and their uses are well known in the art (See, e.g., Berge et al. (1977) “Pharmaceutical Salts”, J. Pharm. Sci. 66:1-19). The prodrugs can be prepared in situ during the final isolation and purification of the compounds, or by separately reacting the purified compound in its free acid form or hydroxyl with a suitable esterifying agent. Hydroxyl groups can be converted into esters via treatment with a carboxylic acid. Examples of prodrug moieties include substituted and unsubstituted, branch or unbranched lower alkyl ester moieties, (e.g., propionoic acid esters), lower alkenyl esters, di-lower alkyl-amino lower-alkyl esters (e.g., dimethylaminoethyl ester), acylamino lower alkyl esters (e.g., acetyloxymethyl ester), acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, di-lower alkyl amides, and hydroxy amides. Preferred prodrug moieties are propionoic acid esters and acyl esters. Prodrugs which are converted to active forms through other mechanisms in vivo are also included.

The language “a prophylactically effective amount” of a compound refers to an amount of a compound of the invention any formula herein or otherwise described herein which is effective, upon single or multiple dose administration to the patient, in preventing or treating a disease or condition.

The language “reduced off-target effects” is intended to include a reduction in any undesired side effect elicited by a compound of the invention when administered in vivo. In some embodiments, a compound described herein has little to no cardio and/or pulmonary toxicity (e.g., when administered to a subject). In some embodiments, a compound described herein has little to no hallucinogenic activity (e.g., when administered to a subject).

The term “sulfhydryl” or “thiol” means —SH.

The term “selective” means a greater activity against a first target (e.g., a 5-HT receptor subtype) relative to a second target (e.g., a second 5-HT receptor subtype). In some embodiments a compound has a selectivity of at least 1.25-fold, at least 1.5 fold, at least 2-fold, at least 3-fold, at least 4-fold, at least 5-fold, at least 6-fold, at least 10-fold or at least 100-fold greater towards a first target relative to a second target. In some embodiments, a compound described herein is selective towards the 5-HT_{2C} receptor relative to one or more other 5-HT receptor subtypes such as 5-HT_{2A} and/or 5-HT_{2B}, preferably 5-HT_{2A}.

The term “subject” includes organisms which are capable of suffering from a serotonin-receptor-related disorder or who could otherwise benefit from the administration of a compound of the invention of the invention, such as human and non-human animals. Preferred humans include human patients suffering from or prone to suffering from a serotonin-related disorder or associated state, as described herein. The term “non-human animals” of the invention includes all vertebrates, e.g., mammals, e.g., rodents, e.g., mice, and non-mammals, such as non-human primates, e.g., sheep, dog, cow, chickens, amphibians, reptiles, etc.

The phrases “systemic administration,” “administered systemically”, “peripheral administration” and “administered peripherally” as used herein mean the administration of a compound of the invention(s), drug or other material, such that it enters the patient's system and, thus, is subject to metabolism and other like processes, for example, subcutaneous administration.

With respect to the nomenclature of a chiral center, terms “R” and “S” configuration are as defined by the IUPAC Recommendations. As to the use of the terms, diastereomer, racemate, epimer and enantiomer will be used in their normal context to describe the stereochemistry of preparations.

### 2. Compounds of the Invention

In one aspect, the invention provides a compound represented by the structural formula I:

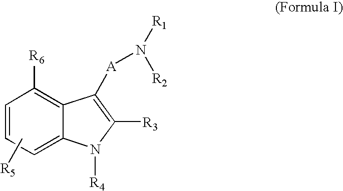

in which

A is C₁-C₄ alkylene, C₂-C₄ alkenylene, or C₂-C₄ alkynylene;

R₁ and R₂ are, independently for each occurrence, H, C₁-C₈ alkyl, C₂-C₈ alkenyl, or C₂-C₈ alkynyl, wherein the C₁-C₈ alkyl, C₂-C₈ alkenyl, or C₂-C₈ alkynyl are optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH;

R₃ is H, C₁-C₈ alkyl, C₂-C₈ alkenyl, or C₂-C₈ alkynyl, wherein the C₁-C₈ alkyl, C₂-C₈ alkenyl, or C₂-C₈ alkynyl are optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH; or R₃ is selected from the group consisting of halogen, C₁-C₈ alkylsulfonyl, formyl, COOH, hydroxy, C₁-C₈ alkoxyl, —SH, thio(C₂-C₈)alkyl, amino, C₁-C₈ alkylamino, and di(C₁-C₈ alkyl)amino;

R₄ is C₁-C₈ alkyl, C₂-C₈ alkenyl, or C₂-C₈ alkynyl, each of which is optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH; or R₄ is selected from the group consisting of C₁-C₈ alkylsulfonyl, formyl, hydroxy, C₁-C₈ alkoxyl, —SH, thio(C₂-C₈)alkyl, amino, C₁-C₈ alkylamino, and di(C₁-C₈ alkyl)amino;

R₅ represents 1-3 substituents, each of which is independently selected from the group consisting of C₁-C₈ alkyl, C₂-C₈ alkenyl, and C₂-C₈ alkynyl, each of which is optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, and di(C₁-C₈ alkyl)amino; or

R₅ represents 1-3 substituents, each of which is independently selected from the group consisting of halogen, C₁-C₈ alkylsulfonyl, formyl, COOH, hydroxy, C₁-C₈ alkoxyl, —SH, thio(C₂-C₈)alkyl, amino, C₁-C₈ alkylamino, and di(C₁-C₈ alkyl)amino;

R₆ is F or OR₇; and

R₇ is C₁-C₈ alkyl, optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH;

or a pharmaceutically acceptable salt thereof.

In another aspect, the invention provides a compound represented by the structural formula II:

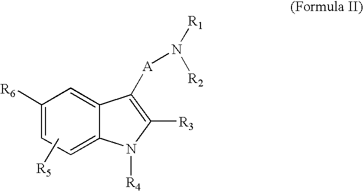

in which

A is C₁-C₄ alkylene, C₂-C₄ alkenylene, or C₂-C₄ alkynylene;

R₁ and R₂ are, independently for each occurrence, H, C₁-C₈ alkyl, C₂-C₈ alkenyl, or C₂-C₈ alkynyl, wherein the C₁-C₈ alkyl, C₂-C₈ alkenyl, or C₂-C₈ alkynyl are optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH;

R₃ is H, C₁-C₈ alkyl, C₂-C₈ alkenyl, or C₂-C₈ alkynyl, wherein the C₁-C₈ alkyl, C₂-C₈ alkenyl, or C₂-C₈ alkynyl are optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH; or R₃ is selected from the group consisting of halogen, C₁-C₈ alkylsulfonyl, formyl, COOH, hydroxy, C₁-C₈ alkoxyl, —SH, thio(C₂-C₈)alkyl, amino, C₁-C₈ alkylamino, and di(C₁-C₈ alkyl)amino;

R₄ is C₁-C₈ alkyl, C₂-C₈ alkenyl, or C₂-C₈ alkynyl, each of which is optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH; or R₄ is selected from the group consisting of C₁-C₈ alkylsulfonyl, formyl, hydroxy, C₁-C₈ alkoxyl, —SH, thio(C₂-C₈)alkyl, amino, C₁-C₈ alkylamino, and di(C₁-C₈ alkyl)amino;

R₅ represents 1-3 substituents, each of which is independently selected from the group consisting of C₁-C₈ alkyl, C₂-C₈ alkenyl, and C₂-C₈ alkynyl, each of which is optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, and di(C₁-C₈ alkyl)amino; or

R₅ represents 1-3 substituents, each of which is independently selected from the group consisting of halogen, C₁-C₈ alkylsulfonyl, formyl, COOH, hydroxy, C₁-C₈ alkoxyl, —SH, thio(C₂-C₈)alkyl, amino, C₁-C₈ alkylamino, and di(C₁-C₈ alkyl)amino;

R₆ is OP(O)(OH)₂, OH, OC(O)R₇, OSO₂OH, SO₂NH₂ or OR₇; and

R₇ is C₁-C₈ alkyl, optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH; phenyl, aralkyl or benzyl;

provided that if R₅ is hydroxy, then R₆ is not hydroxy or alkoxy;

or a pharmaceutically acceptable salt thereof.

In another aspect, the invention provides a compound represented by the structural formula III:

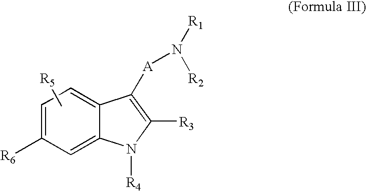

in which

A is C₁-C₄ alkylene, C₂-C₄ alkenylene, or C₂-C₄ alkynylene;

R₁ and R₂ are, independently for each occurrence, H, C₁-C₈ alkyl, C₂-C₈ alkenyl, or C₂-C₈ alkynyl, wherein the C₁-C₈ alkyl, C₂-C₈ alkenyl, or C₂-C₈ alkynyl are optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH;

R₃ is H, C₁-C₈ alkyl, C₂-C₈ alkenyl, or C₂-C₈ alkynyl, wherein the C₁-C₈ alkyl, C₂-C₈ alkenyl, or C₂-C₈ alkynyl are optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH; or R₃ is selected from the group consisting of halogen, C₁-C₈ alkylsulfonyl, formyl, COOH, hydroxy, C₁-C₈ alkoxyl, —SH, thio(C₂-C₈)alkyl, amino, C₁-C₈ alkylamino, and di(C₁-C₈ alkyl)amino;

R₄ is C₁-C₈ alkyl, C₂-C₈ alkenyl, or C₂-C₈ alkynyl, each of which is optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH; or R₄ is selected from the group consisting of C₁-C₈ alkylsulfonyl, formyl, hydroxy, C₁-C₈ alkoxyl, —SH, thio(C₂-C₈)alkyl, amino, C₁-C₈ alkylamino, and di(C₁-C₈ alkyl)amino;

R₅ represents 1-3 substituents, each of which is independently selected from the group consisting of C₁-C₈ alkyl, C₂-C₈ alkenyl, and C₂-C₈ alkynyl, each of which is optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, and di(C₁-C₈ alkyl)amino; or

R₅ represents 1-3 substituents, each of which is independently selected from the group consisting of halogen, C₁-C₈ alkylsulfonyl, formyl, COOH, hydroxy, C₁-C₈ alkoxyl, —SH, thio(C₂-C₈)alkyl, amino, C₁-C₈ alkylamino, and di(C₁-C₈ alkyl)amino;

R₆ is OP(O)(OH)₂, OH, OC(O)R₇, OSO₂OH, SO₂NH₂ or OR₇; and

R₇ is C₁-C₈ alkyl, optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH; phenyl, aralkyl or benzyl;

or a pharmaceutically acceptable salt thereof.

In another aspect, the invention provides a compound represented by the structural formula IV:

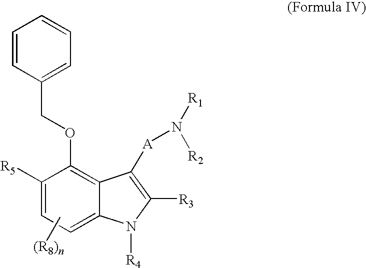

in which

A is C₁-C₄ alkylene, C₂-C₄ alkenylene, or C₂-C₄ alkynylene;

R₁ and R₂ are, independently for each occurrence, H, C₁-C₈ alkyl, C₂-C₈ alkenyl, or C₂-C₈ alkynyl, wherein the C₁-C₈ alkyl, C₂-C₈ alkenyl, or C₂-C₈ alkynyl are optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH;

R₃ is H, C₁-C₈ alkyl, C₂-C₈ alkenyl, or C₂-C₈ alkynyl, wherein the C₁-C₈ alkyl, C₂-C₈ alkenyl, or C₂-C₈ alkynyl are optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH; or R₃ is selected from the group consisting of halogen, C₁-C₈ alkylsulfonyl, formyl, COOH, hydroxy, C₁-C₈ alkoxyl, —SH, thio(C₂-C₈)alkyl, amino, C₁-C₈ alkylamino, and di(C₁-C₈ alkyl)amino;

R₄ is C₁-C₈ alkyl, C₂-C₈ alkenyl, or C₂-C₈ alkynyl, each of which is optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH; or R₄ is selected from the group consisting of C₁-C₈ alkylsulfonyl, formyl, hydroxy, C₁-C₈ alkoxyl, —SH, thio(C₂-C₈)alkyl, amino, C₁-C₈ alkylamino, and di(C₁-C₈ alkyl)amino;

R₅ is C₁-C₈ alkyl, C₂-C₈ alkenyl, or C₂-C₈ alkynyl, each of which is optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, and di(C₁-C₈ alkyl)amino; or

R₅ is halogen, C₁-C₈ alkylsulfonyl, formyl, COOH, hydroxy, C₁-C₈ alkoxyl, —SH, thio(C₂-C₈)alkyl, amino, C₁-C₈ alkylamino, or di(C₁-C₈ alkyl)amino;

R₈ is halogen, C₁-C₈ alkylsulfonyl, formyl, COOH, hydroxy, C₁-C₈ alkoxyl, —SH, thio(C₂-C₈)alkyl, amino, C₁-C₈ alkylamino, or di(C₁-C₈ alkyl)amino; and

n is 0, 1 or 2;

or a pharmaceutically acceptable salt thereof.

In another aspect, the invention provides a pharmaceutical composition comprising a compound represented by the structural formula V:

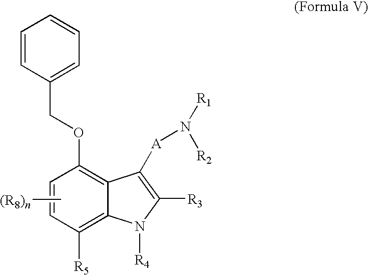

in which

A is C₁-C₄ alkylene, C₂-C₄ alkenylene, or C₂-C₄ alkynylene;

R₁ and R₂ are, independently for each occurrence, H, C₁-C₈ alkyl, C₂-C₈ alkenyl, or C₂-C₈ alkynyl, wherein the C₁-C₈ alkyl, C₂-C₈ alkenyl, or C₂-C₈ alkynyl are optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH;

R₃ is H, C₁-C₈ alkyl, C₂-C₈ alkenyl, or C₂-C₈ alkynyl, wherein the C₁-C₈ alkyl, C₂-C₈ alkenyl, or C₂-C₈ alkynyl are optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH; or R₃ is selected from the group consisting of halogen, C₁-C₈ alkylsulfonyl, formyl, COOH, hydroxy, C₁-C₈ alkoxyl, —SH, thio(C₂-C₈)alkyl, amino, C₁-C₈ alkylamino, and di(C₁-C₈ alkyl)amino;

R₄ is C₁-C₈ alkyl, C₂-C₈ alkenyl, or C₂-C₈ alkynyl, each of which is optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH; or R₄ is selected from the group consisting of C₁-C₈ alkylsulfonyl, formyl, hydroxy, C₁-C₈ alkoxyl, —SH, thio(C₂-C₈)alkyl, amino, C₁-C₈ alkylamino, and di(C₁-C₈ alkyl)amino;

R₅ is C₁-C₈ alkyl, C₂-C₈ alkenyl, or C₂-C₈ alkynyl, each of which is optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, and di(C₁-C₈ alkyl)amino; or

R₅ is halogen, C₁-C₈ alkylsulfonyl, formyl, COOH, hydroxy, C₁-C₈ alkoxyl, —SH, thio(C₂-C₈)alkyl, amino, C₁-C₈ alkylamino, or di(C₁-C₈ alkyl)amino;

R₈ is halogen, C₁-C₈ alkylsulfonyl, formyl, COOH, hydroxy, C₁-C₈ alkoxyl, —SH, thio(C₂-C₈)alkyl, amino, C₁-C₈ alkylamino, or di(C₁-C₈ alkyl)amino; and

n is 0, 1 or 2;

or a pharmaceutically acceptable salt thereof.

In another aspect, the invention provides a pharmaceutical composition comprising a compound represented by the structural formula VI:

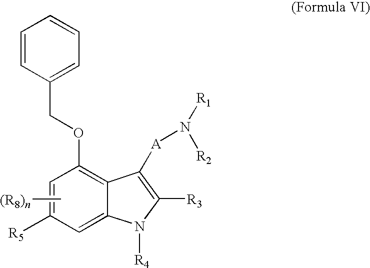

in which

A is C₁-C₄ alkylene, C₂-C₄ alkenylene, or C₂-C₄ alkynylene;

R₁ and R₂ are, independently for each occurrence, H, C₁-C₈ alkyl, C₂-C₈ alkenyl, or C₂-C₈ alkynyl, wherein the C₁-C₈ alkyl, C₂-C₈ alkenyl, or C₂-C₈ alkynyl are optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH;

R₃ is hydrogen, C₁-C₈ alkyl, C₂-C₈ alkenyl, or C₂-C₈ alkynyl, wherein the C₁-C₈ alkyl, C₂-C₈ alkenyl, or C₂-C₈ alkynyl are optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH; or R₃ is selected from the group consisting of halogen, C₁-C₈ alkylsulfonyl, formyl, COOH, hydroxy, C₁-C₈ alkoxyl, —SH, thio(C₂-C₈)alkyl, amino, C₁-C₈ alkylamino, and di(C₁-C₈ alkyl)amino;

R₄ is C₁-C₈ alkyl, C₂-C₈ alkenyl, or C₂-C₈ alkynyl, each of which is optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH; or R₄ is selected from the group consisting of C₁-C₈ alkylsulfonyl, formyl, hydroxy, C₁-C₈ alkoxyl, —SH, thio(C₂-C₈)alkyl, amino, C₁-C₈ alkylamino, and di(C₁-C₈ alkyl)amino;

R₅ is C₁-C₈ alkyl, C₂-C₈ alkenyl, or C₂-C₈ alkynyl, each of which is optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, and di(C₁-C₈ alkyl)amino; or

R₅ is halogen, C₁-C₈ alkylsulfonyl, formyl, COOH, hydroxy, C₁-C₈ alkoxyl, —SH, thio(C₂-C₈)alkyl, amino, C₁-C₈ alkylamino, or di(C₁-C₈ alkyl)amino;

R₈ is halogen, C₁-C₈ alkylsulfonyl, formyl, COOH, hydroxy, C₁-C₈ alkoxyl, —SH, thio(C₂-C₈)alkyl, amino, C₁-C₈ alkylamino, or di(C₁-C₈ alkyl)amino; and

n is 0, 1 or 2;

or a pharmaceutically acceptable salt thereof.

In another aspect, the invention provides a compound represented by the structural formula VII:

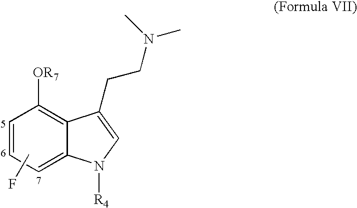

wherein:

F is in at least one of the 5, 6, or 7, positions; and

R₄ and R₇ are independently selected from C₁-C₈ alkyl;

or a pharmaceutically acceptable salt thereof.

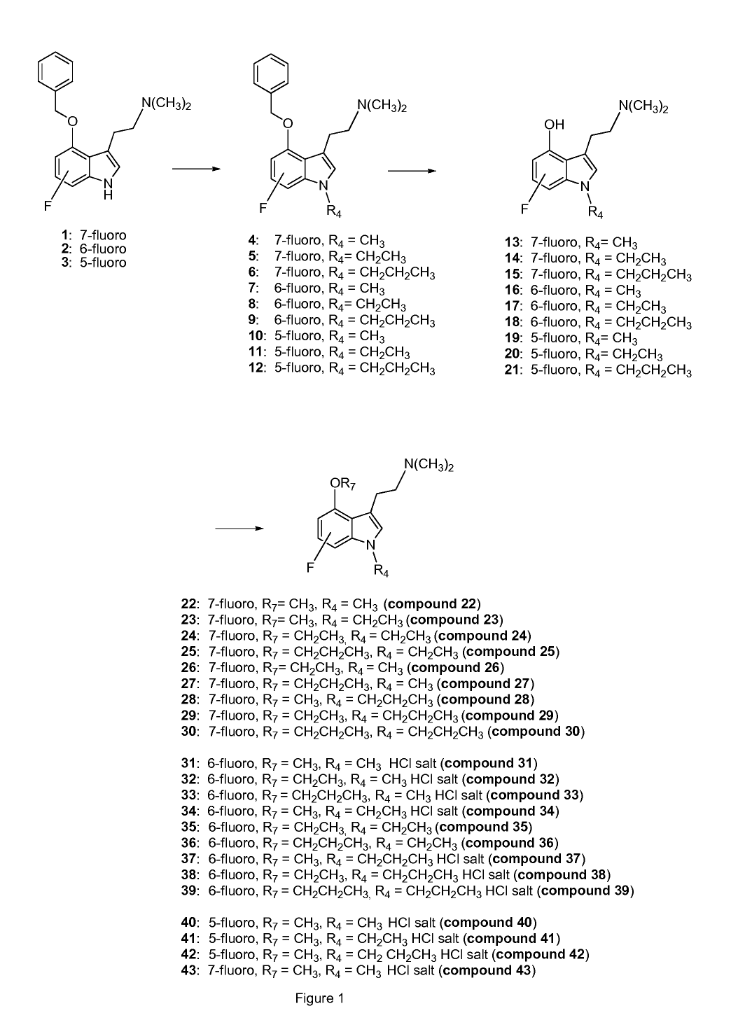

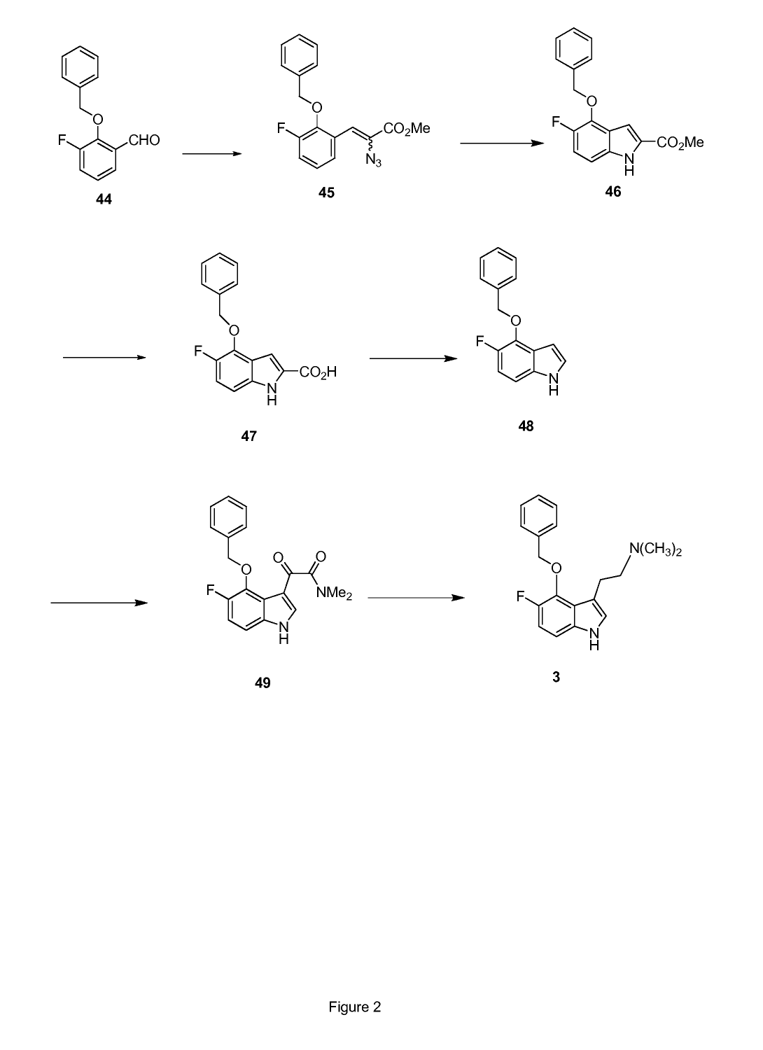

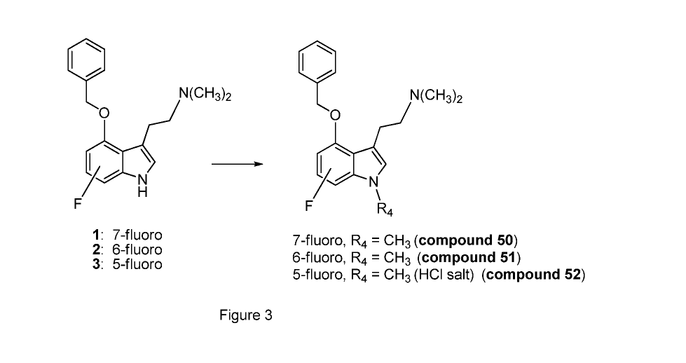

The compounds of the invention can be prepared by a variety of methods, some of which are known in the art or will be apparent to the skilled artisan in light of the present specification. For example, referring to FIG. 1, N-unsubstituted indoles such as compounds 1-3 can be N-alkylated, for example, with an alkyl halide, using a base such as sodium hydride, to give N-alkylated indoles such as compounds 4-12. These 4-substituted indoles can be hydrogenated using, for example, palladium hydroxide on carbon as catalyst in the presence of hydrogen gas, to give 4-hydroxyindoles such as 13-21. The 4-hydroxyindoles can be O-alkylated, for example, with an alkyl halide, using a base such as sodium hydride, to give 4-O-alkoxy-N-substituted indoles such as 22-43. Also, referring to FIG. 2, N-unsubstituted indoles such as 3 can be prepared starting from 3-fluoro-2-benzyloxy benzaldehyde (44) via cyclization of the intermediate styrylazide 45 (prepared using methyl azidoacetate and a base such as sodium methoxide) to the ester 46, followed by hydrolysis, with for example, sodium hydroxide, decarboxylation using as example a copper catalyst and 2-phenylpyridine, oxamidation with oxalyl chloride followed by a secondary amine, and then reduction with LAH in a fashion similar to that reported for compounds 1 and 2 (Blair, J., Kurrasch-Orbaugh, D., Marona-Lewicka, D., Cumbay, M., Watts, V., Barker, E., Nichols, D. “Effect of Ring Fluorination of Hallucinogenic Tryptamines” J. Med. Chem. 2000, 43, 4701).

The compounds and structures depicted herein include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this invention. Such compounds are useful, for example, as analytical tools or probes in biological assays.

Naturally occurring or synthetic isomers can be separated in several ways known in the art. Methods for separating a racemic mixture of two enantiomers include chromatography using a chiral stationary phase (see, e.g., “Chiral Liquid Chromatography,” W. J. Lough, Ed. Chapman and Hall, New York (1989)). Enantiomers can also be separated by classical resolution techniques. For example, formation of diastereomeric salts and fractional crystallization can be used to separate enantiomers. For the separation of enantiomers of carboxylic acids, the diastereomeric salts can be formed by addition of enantiomerically pure chiral bases such as brucine, quinine, ephedrine, strychnine, and the like. Alternatively, diastereomeric esters can be formed with enantiomerically pure chiral alcohols such as menthol, followed by separation of the diastereomeric esters and hydrolysis to yield the free, enantiomerically enriched carboxylic acid. For separation of the optical isomers of amino compounds, addition of chiral carboxylic or sulfonic acids, such as camphorsulfonic acid, tartaric acid, mandelic acid, or lactic acid can result in formation of the diastereomeric salts.

### 3. Uses of the Compounds of the Invention

As described herein below, it has now surprisingly been found that the compounds of the invention have serotonin receptor activity, and can be used to treat or prevent conditions associated with serotonin receptor activity. In some embodiments, a compound described herein has agonist activity against a 5-HT receptor with an EC₅₀ of ≦10 μM.

Thus, in one embodiment, the invention provides methods for treating a subject for a serotonin-receptor-related disorder (i.e., a 5-HT receptor related disorder), or preventing a serotonin-receptor-related disorder (i.e., a 5-HT receptor related disorder), by administering to the subject an effective amount of a compound of the invention, such that the serotonin-receptor-related disorder is treated or prevented.

Fourteen distinct 5-HT receptor subtypes exist in seven separate families. There is particular interest in the three receptor subtypes of the 5-HT₂ family, e.g., 5-HT_{2A}, 5-HT_{2B}, and 5-HT_{2C}. In some embodiments, a compound described herein is selective for a particular subtype (e.g., 5-HT_{2C}). For example, a compound described herein, when administered in vitro or in vivo, has an activity of at least 1.25-fold higher against 5-HT_{2C} over another subtype such as 5-HT_{2A} or 5-HT_{2B} (e.g., at least 1.25-fold, at least 1.5 fold, at least 2-fold, at least 3-fold, at least 4-fold, at least 5-fold, at least 6-fold, at least 10-fold or at least 100-fold). In some embodiments a compound described herein has agonist activity against 5-HT_{2C} with an EC₅₀ of ≦10 μM.

Thus, a compound described herein may be used in the treatment or prevention of disorders such as obesity; a disorder wherein appetite suppression is desirable; a disorder in which treating weight gain is desirable; a disorder in which cognitive enhancement is desirable; depressive disorders (e.g., depression, atypical depression, major depressive disorder, dysthymic disorder, and substance-induced mood disorder); bipolar disorders (e.g., bipolar I disorder, bipolar II disorder, and cyclothymic disorder); anxiety disorders (e.g., panic attack, agoraphobia, panic disorder, specific phobia, social phobia, obsessive compulsive disorder, posttraumatic stress disorder, acute stress disorder, generalized anxiety disorder, separation anxiety disorder, and substance-induced anxiety disorder); mood episodes (e.g., major depressive episode, manic episode, mixed episode, and hypomanic episode); adjustment disorders (e.g., adjustment disorder with anxiety and/or depressed mood); intellectual deficit disorders (e.g., dementia, Alzheimer's disease, and memory deficit); eating disorders (e.g., hyperphagia, bulimia or anorexia nervosa); schizophrenia (e.g., paranoid type, disorganized type, catatonic type, and undifferentiated type); schizophreniform disorder, schizoaffective disorder, delusional disorder, other psychotic disorders (e.g., substance-induced psychotic disorder, L-DOPA-induced psychosis, psychosis associated with Alzheimer's dementia, psychosis associated with Parkinson's disease, psychosis associated with Lewy body disease); sleep disorders (e.g., sleep apnea); suicidal behaviors; sexual dysfunction; migraine; cephalic pain or other pain; raised intracranial pressure; epilepsy; personality disorders; age-related behavioral disorders; behavioral disorders associated with dementia; organic mental disorders; mental disorders in childhood; aggressivity; age-related memory disorders; chronic fatigue syndrome; addiction (e.g., drug and alcohol addiction); premenstrual tension; damage of the central nervous system such as by trauma, stroke, neurodegenerative diseases or toxic or infective CNS diseases such as encephalitis or meningitis; cardiovascular disorders (e.g., thrombosis); hypertension; hyperlipidemia; arterial constriction; osteoarthritis; gall bladder disease; gout; gastrointestinal disorders (e.g., dysfunction of gastrointestinal motility); diabetes mellitus (e.g., Type 2 diabetes mellitus) and diabetes insipidus; cancer; infertility; early mortality; spinal cord injuries; Tourette's syndrome; trichotillomania; other central nervous system disorders; attention deficit hyperactivity disorder (ADHD); canine veterinary diseases (e.g., acral lick dermatitis); and combinations of these disorders that may be present in a mammal. A compound described herein may also be used to suppress appetite in a subject, to enhance cognition in a subject, or treat weight gain in a subject.

In one embodiment, a method of treating a subject suffering from or susceptible to a serotonin-receptor-related disorder includes administering to a subject in need thereof a therapeutically effective amount of a compound of the invention, to thereby treat the subject suffering from or susceptible to a serotonin-receptor-related disorder.

A further aspect relates to a method of treating a subject suffering from or susceptible to obesity, including administering to the subject an effective amount of a compound of the invention to thereby treat the subject suffering from or susceptible to obesity.

A further aspect relates to a method of suppressing appetite in a subject, including administering to the subject an effective amount of a compound of the invention to thereby suppress appetite in the subject.

A further aspect relates to treating weight gain in a subject (e.g., weight gain associated with treatment with another medication), including administering to the subject an effective amount of a compound of the invention to thereby treat weight gain in the subject.

A further aspect relates to enhancing cognition in a subject, including administering to the subject an effective amount of a compound of the invention to thereby enhance cognition in the subject.

A further aspect relates to treating suicidal behavior in a subject, including administering to the subject an effective amount of a compound of the invention to thereby treat suicidal behavior in the subject.

A further aspect relates to a method of treating a subject suffering from or susceptible to Obsessive Compulsive Disorder (OCD), including administering to the subject an effective amount of a compound of the invention to thereby treat the subject suffering from or susceptible to OCD.

A further aspect relates to a method of treating a subject suffering from or susceptible to schizophrenia or psychosis, including administering to the subject an effective amount of a compound of the invention to thereby treat the subject suffering from or susceptible to schizophrenia or psychosis.

A further aspect relates to a method of treating a subject suffering from or susceptible to anxiety or depression, including administering to the subject an effective amount of a compound of the invention to thereby treat the subject suffering from or susceptible to anxiety or depression.

A further aspect relates to a method of treating a subject suffering from or susceptible to migraine, including administering to the subject an effective amount of a compound of the invention to thereby treat the subject suffering from or susceptible to migraine.

In certain embodiments, the methods of the invention include administering to a subject a therapeutically effective amount of a compound of the invention in combination with another pharmaceutically active compound. Examples of pharmaceutically active compounds include compounds known to treat serotonin-related diseases. Other pharmaceutically active compounds that may be used can be found in Harrison's Principles of Internal Medicine, Sixteenth Edition, Eds. D. L. Kasper et al. McGraw-Hill Professional, N.Y., NY (2004); and the 2005 Physician's Desk Reference 59th Edition Thomson Healthcare, 2004, the complete contents of which are expressly incorporated herein by reference. The compound of the invention and the pharmaceutically active compound may be administered to the subject in the same pharmaceutical composition or in different pharmaceutical compositions (at the same time or at different times).

Determination of a therapeutically effective or a prophylactically effective amount of the compound of the invention, can be readily made by the physician or veterinarian (the “attending clinician”), as one skilled in the art, by the use of known techniques and by observing results obtained under analogous circumstances. The dosages may be varied depending upon the requirements of the patient in the judgment of the attending clinician; the severity of the condition being treated and the particular compound being employed. In determining the therapeutically effective amount or dose, and the prophylactically effective amount or dose, a number of factors are considered by the attending clinician, including, but not limited to: the specific serotonin-receptor-related disorder involved; pharmacodynamic characteristics of the particular agent and its mode and route of administration; the desired time course of treatment; the species of mammal; its size, age, and general health; the specific disease involved; the degree of or involvement or the severity of the disease; the response of the individual patient; the particular compound administered; the mode of administration; the bioavailability characteristics of the preparation administered; the dose regimen selected; the kind of concurrent treatment (i.e., the interaction of the compound of the invention with other co-administered therapeutics); and other relevant circumstances.

Treatment can be initiated with smaller dosages, which are less than the optimum dose of the compound. Thereafter, the dosage may be increased by small increments until the optimum effect under the circumstances is reached. For convenience, the total daily dosage may be divided and administered in portions during the day if desired.

Compounds determined to be effective for the prevention or treatment of serotonin-receptor-related disorders in animals, e.g., dogs, chickens, and rodents, may also be useful in treatment of serotonin-receptor-related disorders in humans. Those skilled in the art of treating serotonin-receptor-related disorders in humans will know, based upon the data obtained in animal studies, the dosage and route of administration of the compound to humans. In general, the dosage and route of administration in humans is expected to be similar to that in animals.

The identification of those patients who are in need of prophylactic treatment for serotonin-receptor-related disorders is well within the ability and knowledge of one skilled in the art. Certain of the methods for identification of patients which are at risk of developing serotonin-receptor-related disorders which can be treated by the subject method are appreciated in the medical arts, such as family history, and the presence of risk factors associated with the development of that disease state in the subject patient. A clinician skilled in the art can readily identify such candidate patients, by the use of, for example, clinical tests, physical examination and medical/family history.

In another aspect, a compound of the invention is packaged in a therapeutically effective amount with a pharmaceutically acceptable carrier or diluent. The composition may be formulated for treating a subject suffering from or susceptible to a serotonin-receptor-related disorder, and packaged with instructions to treat a subject suffering from or susceptible to a serotonin-receptor-related disorder.

In another aspect, the invention provides methods for stimulating or increasing serotonin receptor activity. In one embodiment, a method of increasing serotonin receptor activity (or a serotonin receptor related activity) according to the invention includes contacting cells with a compound capable of increasing serotonin receptor activity. The contacting may be in vitro, e.g., by addition of the compound to a fluid surrounding the cells, for example, to the growth media in which the cells are living or existing. The contacting may also be by directly contacting the compound to the cells. Alternately, the contacting may be in vivo, e.g., by passage of the compound through a subject; for example, after administration, depending on the route of administration, the compound may travel through the digestive tract or the blood stream or may be applied or administered directly to cells in need of treatment.

In another aspect, methods of inhibiting a serotonin-receptor-related disorder in a subject include administering an effective amount of a compound of the invention (e.g., a compound of any of the formulae herein capable of increasing serotonin receptor activity) to the subject. The administration may be by any route of administering known in the pharmaceutical arts. The subject may have a serotonin-receptor-related disorder, may be at risk of developing a serotonin-receptor-related disorder, or may need prophylactic treatment prior to anticipated or unanticipated exposure to a conditions capable of increasing susceptibility to a serotonin-receptor-related disorder.

In one aspect, a method of monitoring the progress of a subject being treated with a serotonin receptor active compound of the invention includes determining the pre-treatment status of the serotonin-receptor-related disorder, administering a therapeutically effective amount of a compound of the invention to the subject, and determining the status of the serotonin-receptor-related disorder after an initial period of treatment, wherein the modulation (e.g., improvement) of the status indicates efficacy of the treatment.

In one aspect, methods of selecting a subject suffering from or susceptible to a serotonin-receptor-related disorder for treatment with a compound of the invention comprise determining the pre-treatment status of the serotonin-receptor-related disorder, administering a therapeutically effective amount of a compound of the invention to the subject, and determining the status (of the serotonin-receptor-related disorder after an initial period of treatment with the compound, wherein the modulation (e.g., improvement) of the status is an indication that the serotonin-receptor-related disorder is likely to have a favorable clinical response to treatment with a compound of the invention.

The subject may be at risk of a serotonin-receptor-related disorder, may be exhibiting symptoms of a serotonin-receptor-related disorder, may be susceptible to a serotonin-receptor-related disorder and/or may have been diagnosed with a serotonin-receptor-related disorder.

The initial period of treatment may be the time in which it takes to establish a stable and/or therapeutically effective blood serum level of the compound, or the time in which it take for the subject to clear a substantial portion of the compound, or any period of time selected by the subject or healthcare professional that is relevant to the treatment.

If the modulation of the status indicates that the subject may have a favorable clinical response to the treatment, the subject may be treated with the compound. For example, the subject can be administered therapeutically effective dose or doses of the compound.

Kits of the invention include kits for treating a serotonin-receptor-related disorder in a subject. The invention also includes kits for assessing the efficacy of a treatment for a serotonin-receptor-related disorder in a subject, monitoring the progress of a subject being treated for a serotonin-receptor-related disorder, selecting a subject with a serotonin-receptor-related disorder for treatment according to the invention, and/or treating a subject suffering from or susceptible to a serotonin-receptor-related disorder. The kit may include a compound of the invention, for example, a compound of any of formula described herein, pharmaceutically acceptable esters, salts, and prodrugs thereof, and instructions for use. The instructions for use may include information on dosage, method of delivery, storage of the kit, etc. The kits may also include reagents, for example, test compounds, buffers, media (e.g., cell growth media), cells, etc. Test compounds may include known compounds or newly discovered compounds, for example, combinatorial libraries of compounds. One or more of the kit of the invention may be packaged together, for example, a kit for assessing the efficacy of a treatment for a serotonin-receptor-related disorder may be packaged with a kit for monitoring the progress of a subject being treated for a serotonin-receptor-related disorder according to the invention.

Certain of the present methods can be performed on cells in culture, e.g. in vitro or ex vivo, or on cells present in an animal subject, e.g., in vivo. Compound of the invention can be initially tested in vitro using cells that express a serotonin receptor (see, e.g., the Examples, infra).

Alternatively, the effects of compound of the invention can be characterized in vivo using animals models.

### 4. Pharmaceutical Compositions

The invention also provides a pharmaceutical composition, comprising an effective amount of a compound of the invention (e.g., a compound capable of treating or preventing a condition as described herein, e.g., a compound of any formula herein or otherwise described herein) and a pharmaceutically acceptable carrier.

In an embodiment, the compound of the invention is administered to the subject using a pharmaceutically-acceptable formulation, e.g., a pharmaceutically-acceptable formulation that provides sustained delivery of the compound of the invention to a subject for at least 12 hours, 24 hours, 36 hours, 48 hours, one week, two weeks, three weeks, or four weeks after the pharmaceutically-acceptable formulation is administered to the subject.

In certain embodiments, these pharmaceutical compositions are suitable for topical or oral administration to a subject. In other embodiments, as described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound.

The phrase “pharmaceutically acceptable” refers to those compound of the inventions of the present invention, compositions containing such compounds, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” includes pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier is “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Compositions containing a compound of the invention(s) include those suitable for oral, nasal, topical (including buccal and sublingual), rectal, vaginal, aerosol and/or parenteral administration. The compositions may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, more preferably from about 10 percent to about 30 percent.

Methods of preparing these compositions include the step of bringing into association a compound of the invention(s) with the carrier and, optionally, one or more accessory ingredients. In general, the formulations are prepared by uniformly and intimately bringing into association a compound of the invention with liquid carriers, or finely divided solid carriers, or both, and then, if necessary, shaping the product.

Compositions of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the invention(s) as an active ingredient. A compound may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered active ingredient moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compound of the invention(s) include pharmaceutically-acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

In addition to inert diluents, the oral compositions can include adjuvants such as wetting agents, emulsifying and suspending agents, sweetening, flavoring, coloring, perfuming and preservative agents.

Suspensions, in addition to the active compound of the invention(s) may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions of the invention for rectal or vaginal administration may be presented as a suppository, which may be prepared by mixing one or more compound of the invention(s) with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent.

Compositions of the present invention which are suitable for vaginal administration also include pessaries, tampons, creams, gels, pastes, foams or spray formulations containing such carriers as are known in the art to be appropriate.

Dosage forms for the topical or transdermal administration of a compound of the invention(s) include powders, sprays, ointments, pastes, creams, lotions, gels, solutions, patches and inhalants. The active compound of the invention(s) may be mixed under sterile conditions with a pharmaceutically-acceptable carrier, and with any preservatives, buffers, or propellants which may be required.

The ointments, pastes, creams and gels may contain, in addition to compound of the invention(s) of the present invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of the invention(s), excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

The compound of the invention(s) can be alternatively administered by aerosol. This is accomplished by preparing an aqueous aerosol, liposomal preparation or solid particles containing the compound. A nonaqueous (e.g., fluorocarbon propellant) suspension could be used. Sonic nebulizers are preferred because they minimize exposing the agent to shear, which can result in degradation of the compound.

Ordinarily, an aqueous aerosol is made by formulating an aqueous solution or suspension of the agent together with conventional pharmaceutically-acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Transdermal patches have the added advantage of providing controlled delivery of a compound of the invention(s) to the body. Such dosage forms can be made by dissolving or dispersing the agent in the proper medium. Absorption enhancers can also be used to increase the flux of the active ingredient across the skin. The rate of such flux can be controlled by either providing a rate controlling membrane or dispersing the active ingredient in a polymer matrix or gel.

Ophthalmic formulations, eye ointments, powders, solutions and the like, are also contemplated as being within the scope of the invention.

Pharmaceutical compositions of the invention suitable for parenteral administration comprise one or more compound of the invention(s) in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers, which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of compound of the invention(s) in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compound of the invention(s) are administered as pharmaceuticals, to humans and animals, they can be given per se or as a pharmaceutical composition containing, for example, 0.1 to 99.5% (more preferably, 0.5 to 90%) of active ingredient in combination with a pharmaceutically-acceptable carrier.

Regardless of the route of administration selected, the compound of the invention(s), which may be used in a suitable hydrated form, and/or the pharmaceutical compositions of the present invention, are formulated into pharmaceutically-acceptable dosage forms by conventional methods known to those of skill in the art.

Actual dosage levels and time course of administration of the active ingredients in the pharmaceutical compositions of the invention may be varied so as to obtain an amount of the active ingredient which is effective to achieve the desired therapeutic response for a particular patient, composition, and mode of administration, without being toxic to the patient. An exemplary dose range is from 0.1 to 10 mg per day.

A preferred dose of the compound of the invention for the present invention is the maximum that a patient can tolerate and not develop serious side effects. Preferably, the compound of the present invention is administered at a concentration or amount of about 0.001 mg to about 100 mg per kilogram of body weight, about 0.01-about 50 mg/kg or about 10 mg-about 30 mg/kg of body weight. Ranges intermediate to the above-recited values are also intended to be part of the invention.

The invention is further illustrated by the following examples which are intended to illustrate but not limit the scope of the invention.

### EXAMPLES

### Chemical Synthesis

General. All exemplified target compounds are fully analyzed and characterized (mp, TLC, CHN, MS, ¹H-NMR) prior to submission for biological evaluation. Melting points are uncorrected. Thin-layer chromatography was carried out on Merck Si 250F plates. Visualization was accomplished with ultraviolet exposure or with phosphomolybdic acid. Flash chromatography was carried out on silica gel (60 μM). Elemental analyses were performed at Atlantic Microlab. MS were carried out on a Agilent 1100 series HPLC-Mass Spectrometer. ¹H and ¹³C NMR spectra were recorded at 300 and 75 MHz, respectively, on a Jeol Eclipse 300 Spectrometer. NMR assignments are based on a combination of the ¹H, ¹³C, ¹H COSY, HMBC and HMQC spectra. Coupling constants are given in hertz (Hz). Anhydrous methylene chloride, tetrahydrofuran, and dimethylformamide are Aldrich Sure/Seal™, and other materials are reagent grade.

### Example 1

### 2-(7-Fluoro-4-methoxy-1-methyl-1H-indol-3-yl)-N,N-dimethylethanamine (Compound 22)

### Step One: 4-Benzyloxy-3-(2-dimethylaminoethyl)-7-fluoro-1-methyl-1H-indole (Compound 4)

At 0° C., NaH (2 eq) was added to a solution of 1 eq of compound 1 (prepared according to J. Med. Chem. 2000, 43, 4701) in DMF. After 30 min, a solution of 0.1 M methyl iodide in DMF was added slowly over 30 min, and then the reaction mixture was allowed to warm to 25° C. and stirred at that temperature for 1 h. After standard workup, the brown oil residue was purified by column chromatography, eluting with CH₂Cl₂/2% NH₄OH. A brown oil was obtained in 84% yield. ¹H NMR (CDCl₃, 300 MHz) δ 2.13 (s, 6H), 2.50-2.56 (m, 2H), 2.95-3.01 (m, 2H), 3.89 (d, 3H, J=1.9), 5.13 (s, 2H), 6.29 (dd, 1H, J=8.5, 2.8), 6.67 (dd, 1H, J=8.5, 12.1), 6.69 (s, 1H), 7.29-7.40 (m, 3H), 7.45-7.48 (m, 2H). APCI [M+1]: 327.2.

### Step Two: 3-(2-Dimethylaminoethyl)-7-fluoro-1-methyl-1H-indol-4-ol (Compound 13)

A mixture of 4 and Pd(OH)₂/C in MeOH was hydrogenated at ambient pressure for 2 h at 25° C., then the mixture was filtered through a plug of Celite and washed with EtOAc. The crude product was purified by column chromatography eluting with CH₂Cl₂/2% NH₄OH to give a white solid in 88% yield. ¹H NMR (CDCl₃, 300 MHz) δ 2.35 (s, 6H), 2.63-2.67 (m, 2H), 2.86-2.89 (m, 2H), 3.87 (d, 3H, J=2.2), 6.33 (dd, 1H, J=8.2, 3.3), 6.61 (s, 1H), 6.69 (dd, 1H, J=8.5, 12.4). APCI [M+1]: 237.2.

### Step Three: 2-(7-Fluoro-4-methoxy-1-methyl-1H-indol-3-yl)-N,N-dimethylethanamine (Compound 22)

At 0° C., NaH (2 eq) was added to a solution of compound 13 (1 eq) in DMF. After 30 min, a solution of 0.1 M methyl iodide in DMF was added slowly over 30 min, and then warmed up to room temperature for 1 h. After standard workup, the brown oil residue was purified by column chromatography, eluting with CH₂Cl₂/2% NH₄OH to give a brown oil in 72% yield. ¹H NMR (CDCl₃, 300 MHz) δ 2.32 (s, 6H), 2.53-2.59 (m, 2H), 2.95-3.02 (m, 2H), 3.02 (s, 3H), 3.88 (d, 3H, J=1.9), 6.23 (dd, 1H, J=8.5, 2.7), 6.68 (s, 1H), 6.70 (dd, 1H, J=8.5, 12.1). APCI [M+1]: 251.2. Elemental analysis: Calc.: C, 67.18; H, 7.65; N, 11.19. Found: C, 67.12; H, 7.64; N, 11.09.

Compound 43 (hydrochloride salt of compound 22) was prepared as described in Example 10, Step 3 as a white solid: mp 259-261° C.; ¹H NMR (300 MHz, DMSO-d₆) δ 2.80 (s, 6H), 3.08-3.15 (m, 2H), 3.18-3.23 (m, 2H), 3.85 (s, 3H), 3.87 (dd, 1H, J=2.2), 6.38 (dd, 1H, J=8.5, 3.0), 6.83 (dd, 1H, J=12.4, 8.5), 7.12 (s, 1H), 10.17 (bs, 1H). APCI [M+1]: 251.1. Elemental analysis: Calc.: C, 57.90; H, 6.98; N, 9.65; Cl, 13.43. Found: C, 57.82; H, 6.92; N, 9.48; Cl, 13.45 (containing 1.1 equivalent HCl).

### Example 2

### 2-(1-Ethyl-7-fluoro-4-methoxy-1H-indol-3-yl)-N,N-dimethylethanamine (Compound 23)

### Step One: 4-Benzyloxy-3-(2-dimethylaminoethyl)-1-ethyl-7-fluoro-1H-indole (Compound 5)

Following the procedure used to prepare compound 4, compound 1 was treated with ethyl iodide to give a white solid in 93% yield. ¹H NMR (CDCl₃, 300 MHz) δ 1.41 (t, 3H, J=7.1), 2.12 (s, 6H), 2.51-2.57 (m, 2H), 2.97-3.03 (m, 2H), 4.23 (q, 2H, J=7.1), 5.13 (s, 2H), 6.31 (dd, 1H, J=8.5, 3.0), 6.69 (dd, 1H, J=8.5, 12.2), 6.76 (s, 1H), 7.26-7.40 (m, 3H), 7.46-7.49 (m, 2H). APCI [M+1]: 341.2.

### Step Two: 3-(2-Dimethylaminoethyl)-1-ethyl-7-fluoro-1H-indol-4-ol (Compound 14)

Following the procedure used to prepare compound 13, compound 5 gave a white solid in 92% yield. ¹H NMR (CDCl₃, 300 MHz) δ 1.41 (t, 3H, J=7.1), 2.36 (s, 6H), 2.64-2.68 (m, 2H), 2.87-2.91 (m, 2H), 4.22 (q, 2H, J=7.1), 6.34 (dd, 1H, J=8.4, 3.4), 6.70 (dd, 1H, J=8.5, 12.4), 6.69 (s, 1H). APCI [M+1]: 251.2.

### Step Three: 2-(1-Ethyl-7-fluoro-4-methoxy-1H-indol-3-yl)-N,N-dimethylethanamine (Compound 23)

Following the procedure used to prepare compound 22, compound 14 gave a brown oil in 64% yield. ¹H NMR (CDCl₃, 300 MHz) δ 1.40 (dt, 3H, J=7.1, 0.5), 2.32 (s, 6H), 2.54-2.59 (m, 2H), 2.97-3.02 (m, 2H), 3.86 (s, 3H), 4.22 (dq, 2H, J=7.1, 0.5), 6.24 (dd, 1H, J=8.5, 2.9), 6.70 (dd, 1H, J=12.4, 8.5), 6.75 (s, 1H). APCI [M+1]: 265.2. Elemental analysis: Calc.: C, 68.16; H, 8.01; N, 10.60. Found: C, 67.99; H, 8.17; N, 10.40.

### Example 3

### 2-(4-Ethoxy-1-ethyl-7-fluoro-1H-indol-3-yl)-N,N-dimethylethanamine (Compound 24)

Following the procedure used to prepare compound 22, compound 14 was treated with ethyl iodide to give a white solid in 94% yield. mp 59-60° C. ¹H NMR (CDCl₃, 300 MHz) δ1.40 (dt, 3H, J=7.1, 0.8), 1.47 (t, 3H, J=6.9), 2.32 (s, 6H), 2.56-2.62 (m, 2H), 2.99-3.05 (m, 2H), 4.09 (q, 2H, J=7.1), 4.22 (dq, 2H, J=7.1, 0.5), 6.23 (dd, 1H, J=8.5, 3.0), 6.68 (dd, 1H, J=8.5, 12.2), 6.75 (s, 1H). APCI [M+1]: 279.2. Elemental analysis: Calc.: C, 69.04; H, 8.33; N, 10.06. Found: C, 68.86; H, 8.38; N, 9.83.

### Example 4

### 2-(1-Ethyl-7-fluoro-4-propoxy-1H-indol-3-yl)-N,N-dimethylethanamine (Compound 25)

Following the procedure used to prepare compound 22, compound 14 was treated with propyl iodide to give a white solid in 75% yield. m.p.: 49-50° C. ¹H NMR (CDCl₃, 300 MHz) δ 1.08 (t, 3H, J=7.1), 1.40 (t, 3H, J=7.1), 1.83-1.94 (m, 2H), 2.31 (s, 6H), 2.51-2.63 (m, 2H), 3.00-3.06 (m, 2H), 3.98 (t, 2H, J=6.6), 4.22 (q, 2H, J=7.1), 6.23 (dd, 1H, J=8.5, 5.8), 6.68 (dd, 1H, J=8.5, 12.4), 6.75 (s, 1H). APCI [M+1]: 293.2. Elemental analysis: Calc.: C, 69.83; H, 8.62; N, 9.58. Found: C, 69.82; H, 8.60; N, 9.45.

### Example 5

### 2-(4-Ethoxy-7-fluoro-1-methyl-1H-indol-3-yl)-N,N-dimethylethanamine (Compound 26)

Following the procedure used to prepare compound 22, compound 13 was treated with ethyl iodide to give a brown oil in 44% yield. ¹H NMR (CDCl₃, 300 MHz) δ 1.47 (t, 3H, J=6.9), 2.31 (s, 6H), 2.56-2.62 (m, 2H), 2.98-3.04 (m, 2H), 3.88 (d, 3H, J=1.9H, 4.09 (q, 2H, J=6.9), 6.22 (dd, 1H, J=8.5, 2.9), 6.67 (dd, 1H, J=12.4, 8.5), 6.68 (s, 1H). APCI [M+1]: 265.2. Elemental analysis: Calc.: C, 68.16; H, 8.01; N, 10.60. Found: C, 68.16; H, 8.13; N, 10.41.

### Example 6

### 2-(7-Fluoro-1-methyl-4-propoxy-1H-indol-3-yl)-N,N-dimethylethanamine (Compound 27)

Following the procedure used to prepare compound 22, compound 13 was treated with propyl iodide to give a green oil in 90% yield. ¹H NMR (CDCl₃, 300 MHz) δ 1.08 (t, 3H, J=7.1), 1.81-1.94 (m, 2H), 2.31 (s, 6H), 2.56-2.62 (m, 2H), 2.99-3.05 (m, 2H), 3.88 (d, 3H, J=1.9), 3.98 (t, 2H, J=6.6), 6.21 (dd, 1H, J=8.5, 2.8), 6.68 (s, 1H), 6.68 (dd, 1H, J=12.1, 8.5). APCI [M+1]: 279.2. Elemental analysis: Calc.: C, 69.04; H, 8.33; N, 10.06. Found: C, 69.23; H, 8.47; N, 10.01.

### Example 7

### 2-(7-Fluoro-4-methoxy-1-propyl-1H-indol-3-yl)-N,N-dimethylethanamine (Compound 28)

### Step One: 4-Benzyloxy-3-(2-dimethylaminoethyl)-7-fluoro-1-propyl-1H-indole (Compound 6)

Following the procedure used to prepare compound 4, compound 1 was treated with propyl iodide to give a brown oil in 85% yield. ¹H NMR (CDCl₃, 300 MHz) δ 0.90 (t, 3H, J=7.4), 1.77-1.86 (m, 2H), 2.13 (s, 6H), 2.52-2.58 (m, 2H), 2.97-3.03 (m, 2H), 4.14 (t, 2H, J=6.9), 5.13 (s, 2H), 6.32 (dd, 1H, J=8.5, 2.7), 6.69 (dd, 1H, J=12.2, 8.5), 6.75 (s, 1H), 7.29-7.41 (m, 3H), 7.47-7.50 (m, 2H). APCI [M+1]: 355.2.

### Step Two: 3-(2-Dimethylaminoethyl)-7-fluoro-1-propyl-1H-indol-4-ol (Compound 15)

Following the procedure used to prepare compound 13, compound 6 gave a white solid in 96% yield. ¹H NMR (CDCl₃, 300 MHz) δ0.89 (t, 3H, J=7.4), 1.75-1.88 (m, 2H), 2.35 (s, 6H), 2.64-2.68 (m, 2H), 2.87-2.91 (m, 2H), 4.11 (t, 2H, J=7.1), 6.34 (dd, 1H, J=8.2, 3.3), 6.68 (s, 1H), 6.70 (dd, 1H, J=12.5, 8.2). APCI [M+1]: 265.2.

### Step Three: 2-(7-Fluoro-4-methoxy-1-propyl-1H-indol-3-yl)-N,N-dimethylethanamine (Compound 28)

Following the procedure used to prepare compound 22, compound 15 gave a brown oil in 70% yield. ¹H NMR (CDCl₃, 300 MHz) δ 0.88 (t, 3H, J=7.4), 1.74-1.86 (m, 2H), 2.32 (s, 6H), 2.53-2.59 (m, 2H), 2.96-3.02 (m, 2H), 3.86 (s, 3H), 4.12 (t, 2H, J=7.1), 6.24 (dd, 1H, J=8.5, 2.7), 6.70 (dd, 1H, J=8.5, 12.1), 6.73 (s, 1H). APCI [M+1]: 279.2. Elemental analysis: Calc.: C, 69.04; H, 8.33; N, 10.06. Found: C, 68.57; H, 8.39; N, 9.87.

### Example 8

### 2-(4-Ethoxy-7-fluoro-1-propyl-1H-indol-3-yl)-N,N-dimethylethanamine (Compound 29)

Following the procedure used to prepare compound 22, compound 15 was treated with ethyl iodide to give a brown oil in 85% yield. ¹H NMR (CDCl₃, 300 MHz) δ 0.88 (t, 3H, J=7.1), 1.47 (t, 3H, J=7.1), 1.74-1.87 (m, 2H), 2.32 (s, 6H), 2.56-2.62 (m, 2H), 2.99-3.05 (m, 2H), 4.09 (q, 2H, J=6.9), 4.12 (t, 2H, J=6.3), 6.22 (dd, 1H, J=8.5, 2.7), 6.68 (dd, 1H, J=12.4, 8.5), 6.73 (s, 1H). APCI [M+1]: 293.2. Elemental analysis: Calc.: C, 69.83; H, 8.62; N, 9.58. Found: C, 70.09; H, 8.78; N, 9.54.

### Example 9

### 2-(7-Fluoro-4-propoxy-1-propyl-1H-indol-3-yl)-N,N-dimethylethanamine (Compound 30)

Following the procedure used to prepare compound 22, compound 15 was treated with propyl iodide to give a brown oil in 100% yield. ¹H NMR (CDCl₃, 300 MHz) δ 0.88 (t, 3H, J=7.4), 1.08 (t, 3H, J=7.4), 1.74-1.94 (m, 4H), 2.31 (s, 6H), 2.56-2.62 (m, 2H), 3.00-3.06 (m, 2H), 3.98 (t, 2H, J=6.6), 4.12 (t, 2H, J=7.1), 6.22 (dd, 1H, J=8.5, 2.9), 6.68 (dd, 1H, J=13.3, 8.5), 6.73 (s, 1H). APCI [M+1]: 307.3. Elemental analysis: Calc.: C, 70.55; H, 8.88; N, 9.14. Found: C, 70.32; H, 9.00; N, 9.00.

### Example 10

### 2-(6-Fluoro-4-methoxy-1-methyl-1H-indol-3-yl)-N,N-dimethylethanamine hydrochloride (Compound 31)

### Step One: 4-Benzyloxy-3-(2-dimethylaminoethyl)-6-fluoro-1-methyl-1H-indole (Compound 7)

Following the procedure used to prepare compound 4, compound 2 (prepared according to J. Med. Chem. 2000, 43, 4701) gave the product in 76% yield. ¹H NMR (300 MHz, CD₃OD) δ2.07 (s, 6H), 2.52-2.57 (m, 2H), 2.85-2.95 (m, 2H), 3.64 (s, 3H), 5.13 (s, 2H), 6.40-6.44 (m, 1H), 6.63-6.66 (m, 1H), 6.80 (s, 1H), 7.31-7.42 (m, 3H), 7.49-7.52 (m, 2H). APCI [M+1]: 327.2.

### Step Two: 3-(2-Dimethylaminoethyl)-6-fluoro-1-methyl-1H-indol-4-ol (Compound 16)

Following the procedure used to prepare compound 13, compound 7 gave the product in 60% yield. ¹H NMR (300 MHz, CD₃OD) δ2.35-2.38 (m, 6H), 2.71-2.75 (m, 2H), 2.94-3.01 (m, 2H), 3.58-3.64 (m, 3H), 6.10-6.15 (m, 1H), 6.44-6.48 (m, 1H), 6.76 (s, 1H). APCI [M+1]: 237.2.

### Step Three: 2-(6-Fluoro-4-methoxy-1-methyl-1H-indol-3-yl)-N,N-dimethylethanamine hydrochloride (Compound 31)

Following the procedure used to prepare compound 22, compound 16 gave the product, after formation of the HCl salt using a 4 M solution of HCl in dioxane, in 31% yield as a white solid: mp 260.6-261.3° C.; ¹H NMR (300 MHz, DMSO-d₆) δ 2.80 (s, 3H). 2.81 (s, 3H), 3.08-3.13 (m, 2H), 3.18-3.21 (m, 2H), 3.65 (s, 3H), 3.89 (s, 3H), 6.47 (dd, 1H, J=11.8, 1.9), 6.87 (dd, 1H, J=9.9, 1.9) 7.07 (s, 1H), 10.17 (br, 1H). APCI [M+1]: 251.2. Elemental analysis: Calc.: C, 58.27; H, 7.06; N, 9.71; Cl, 12.29. Found: C, 58.20; H, 7.04; N, 9.80; Cl, 12.46 (containing 0.1 equivalent H₂O).

### Example 11

### 2-(4-Ethoxy-6-fluoro-1-methyl-1H-indol-3-yl)-N,N-dimethylethanamine hydrochloride (Compound 32)

Following the procedure used to prepare compound 22, compound 16 with ethyl iodide gave the product, after formation of the HCl salt using a 4 M solution of HCl in dioxane, in 88% yield as a white solid: mp 202.2-203.3° C.; ¹H NMR (300 MHz, DMSO-d₆) δ 1.43 (t, 3H, J=6.9), 2.80 (s, 6H), 3.09-3.16 (m, 2H), 3.22-3.27 (m, 2H), 3.65 (s, 3H), 4.13 (q, 2H, J=6.9), 6.45 (dd, 1H, J=12.1, 1.9), 6.86 (dd, 1H, J=9.9, 1.9), 7.08 (s, 1H), 9.84 (br, 1H). APCI [M+1]: 265.2. Elemental analysis: Calc.: C, 57.82; H, 7.50; N, 8.99; Cl, 11.38. Found: C, 57.85; H, 7.45; N, 8.97; Cl, 11.45 (containing 0.6 equivalent H₂O).

### Example 12

### 2-(6-Fluoro-1-methyl-4-propoxy-1H-indol-3-yl)-N,N-dimethylethanamine hydrochloride (Compound 33)

Following the procedure used to prepare compound 22, compound 16 with propyl iodide gave the product, after formation of the HCl salt using a 4 M solution of HCl in dioxane, in 84% yield as a white solid: mp 195.4-196.8° C.; ¹H NMR (300 MHz, DMSO-d₆) δ 1.02 (t, 3H, J=7.41), 1.78-1.90 (m, 2H), 2.79 (s, 6H), 3.11-3.16 (m, 2H), 3.22-3.29 (m, 2H), 3.65 (s, 3H), 4.03 (t, 2H, J=6.6), 6.45 (dd, 1H, J=12.1, 1.9), 6.86 (dd, 1H, J=9.9, 1.9), 7.08 (s, 1H), 10.0 (br, 1H). APCI [M+1]: 279.2. Elemental analysis: Calc.: C, 60.76; H, 7.66; N, 8.86; Cl, 11.66. Found: C, 60.42; H, 7.62; N, 8.89; Cl, 11.63 (containing 1.04 equivalent HCl).

### Example 13

### 2-(1-Ethyl-6-fluoro-4-methoxy-1H-indol-3-yl)-N,N-dimethylethanamine hydrochloride (Compound 34)

### Step One: 4-Benzyloxy-3-(2-dimethylaminoethyl)-6-fluoro-1-ethyl-1H-indole (Compound 8)

Following the procedure used to prepare compound 5, compound 2 (prepared according to J. Med. Chem. 2000, 43, 4701) gave the product in 75% yield. ¹H NMR (300 MHz, CDCl₃) δ 1.39 (t, 3H, J=5.4), 2.12 (s, 6H), 2.52-2.57 (m, 2H), 2.96-3.01 (m, 2H), 3.99 (q, 2H, J=7.1), 5.13 (s, 2H), 6.33 (dd, 1H, J=11.8, 1.9), 6.58 (dd, 1H, J=9.6, 1.9), 6.76 (s, 1H), 7.31-7.41 (m, 3H), 7.46-7.49 (m, 2H). APCI [M+1]: 341.2.

### Step Two: 3-(2-Dimethylaminoethyl)-1-ethyl-6-fluoro-1H-indol-4-ol (Compound 17)

Following the procedure used to prepare compound 13, compound 8 gave the product in 76% yield as a white solid: mp 87.2-88.8° C.; ¹H NMR (300 MHz, CDCl₃) δ 1.39 (t, 3H, J=7.1), 2.37 (s, 6H), 2.67-2.70 (m, 2H), 2.87-2.90 (m, 2H), 3.97 (q, 2H, J=7.1), 6.30 (dd, 1H, J=11.5, 2.2), 6.46 (dd, 1H, J=9.9, 2.2), 6.69 (s, 1H). APCI [M+1]: 251.2. Elemental analysis: Calc.: C, 67.18; H, 7.65; N, 11.19. Found: C, 67.06; H, 7.64; N, 11.10.

### Step Three: 2-(1-Ethyl-6-fluoro-4-methoxy-1H-indol-3-yl)-N,N-dimethylethanamine hydrochloride (Compound 34)

Following the procedure used to prepare compound 22, compound 17 gave the product, after formation of the HCl salt using a 4 M solution of HCl in dioxane, in 46% yield as a white solid: mp 233.4-234.9° C.; ¹H NMR (300 MHz, DMSO-d₆) δ 1.29 (t, 3H, J=7.1), 2.81 (s, 6H), 3.08-3.14 (m, 2H), 3.18-3.25 (m, 2H), 3.88 (s, 3H), 4.06 (q, 2H, J=7.1), 6.46 (dd, 1H, J=11.8, 1.9), 6.92 (dd, 1H, J=9.9, 1.9), 7.13 (s, 1H), 10.12 (br, 1H). APCI [M+1]: 265.1. Elemental analysis: Calc.: C, 59.89; H, 7.37; N, 9.31; Cl, 11.79. Found: C, 59.60; H, 7.33; N, 9.10; Cl, 11.88.

### Example 14

### 2-(4-Ethoxy-1-ethyl-6-fluoro-1H-indol-3-yl)-N,N-dimethylethanamine (Compound 35)

Following the procedure used to prepare compound 22, compound 17 was treated with ethyl iodide to give the product in 83% yield as a white solid: mp 77.1-78.2° C.; ¹H NMR (300 MHz, DMSO-d₆) δ 1.38 (t, 3H, J=7.2), 1.48 (t, 3H, J=6.8), 2.31 (s, 6H), 2.56-2.61 (m, 2H), 2.98-3.03 (m, 2H), 3.98 (q, 2H, J=7.1), 4.11 (q, 2H, J=6.9), 6.24 (dd, 1H, J=11.8, 1.9), 6.56 (dd, 1H, J=9.63, 2.2), 6.75 (s, 1H). APCI [M+1]: 279.2. Elemental analysis: Calc.: C, 68.15; H, 8.36; N, 9.93. Found: C, 68.42; H, 8.27; N, 9.91 (containing 0.2 equivalent H₂O).

### Example 15

### 2-(1-Ethyl-6-fluoro-4-propoxy-1H-indol-3-yl)-N,N-dimethylethanamine (Compound 36)

Following the procedure used to prepare compound 22, compound 17 was treated with propyl iodide to give the product in 71% yield as a white solid: mp 68.9-70.1° C.; ¹H NMR (300 MHz, DMSO-d₆) δ 1.08 (t, 3H, J=7.4), 1.38 (t, 3H, J=7.1), 1.83-1.95 (m, 2H), 2.57-2.62 (m, 2H), 2.31 (s, 6H), 2.99-3.04 (m, 2H), 3.98 (t, 2H, J=6.3), 3.99 (t, 2H, J=7.4), 6.24 (dd, 1H, J=11.8, 1.9), 6.55 (dd, 1H, J=9.6, 1.9), 6.74 (s, 1H). APCI [M+1]: 293.2. Elemental analysis: Calc.: C, 69.40; H, 8.63; N, 9.52. Found: C, 69.51; H, 8.65; N, 9.42 (containing 0.1 equivalent H₂O).

### Example 16

### 2-(6-Fluoro-4-methoxy-1-propyl-1H-indol-3-yl)-N,N-dimethylethanamine hydrochloride (Compound 37)

### Step One: 4-Benzyloxy 3-(2-dimethylaminoethyl)-6-fluoro-1-propyl-1H-indole (Compound 9)

Following the procedure used to prepare compound 6, compound 2 (prepared according to J. Med. Chem. 2000, 43, 4701) gave the product in 86% yield. ¹H NMR (300 MHz, CDCl₃) δ0.91 (t, 3H, J=7.2), 1.75-1.83 (m, 2H), 2.12 (s, 6H), 2.53-2.59 (m, 2H), 2.98-3.02 (m, 2H), 3.90 (t, 2H, J=7.1), 5.13 (s, 2H), 6.30 (dd, 1H, J=11.5, 1.9), 6.58 (dd, 1H, J=9.6, 2.2), 6.75 (s, 1H), 7.32-7.41 (m, 3H), 7.47-7.50 (m, 2H). APCI [M+1]: 355.2.

### Step Two: 3-(2-Dimethylaminoethyl)-6-fluoro-1-propyl-1H-indol-4-ol (Compound 18)

Following the procedure used to prepare compound 13, compound 9 gave the product in 86% yield as a white solid: mp 106.3-107.8° C.; ¹H NMR (300 MHz, CDCl₃) δ 0.91 (t, 3H, J=7.1), 1.76-1.83 (m, 2H), 2.37 (s, 6H), 2.66-2.70 (m, 2H), 2.86-2.89 (m, 2H), 3.87 (t, 2H, J=7.3), 6.30 (dd, 1H, J=11.5, 2.2), 6.46 (dd, 1H, J=9.9, 2.2), 6.67 (s, 1H). APCI [M+1]: 265.2. Elemental analysis: Calc: C, 68.16; H, 8.01; N, 10.60. Found: C, 68.00; H, 8.05; N, 10.47.

### Step Three: 2-(6-Fluoro-4-methoxy-1-propyl-1H-indol-3-yl)-N,N-dimethylethanamine hydrochloride (Compound 37)

Following the procedure used to prepare compound 22, compound 18 gave the product, after formation of the HCl salt using 4 M solution of HCl in dioxane, in 25% yield as a white solid: mp 119.8-121.3° C.; ¹H NMR (300 MHz, DMSO-d₆) δ 0.80 (t, 3H, J=7.4), 1.64-1.76 (m, 2H), 2.79 (s, 3H), 2.80 (s, 3H), 3.09-3.14 (m, 2H), 3.19-3.24 (m, 2H), 3.89 (s, 3H), 3.99 (t, 2H, J=6.9), 6.45 (dd, 1H, J=11.8, 1.9), 6.92 (dd, 1H, J=10.2, 1.9), 7.11 (s, 1H), 10.4 (br, 1H). APCI [M+1]: 279.1. Elemental analysis: Calc.: C, 59.00; H, 7.66; N, 8.60; Cl, 12.52. Found: C, 58.92; H, 7.62; N, 8.51; Cl, 12.32 (containing 1.15 equivalent HCl and 0.3 equivalent H₂O).

### Example 17

### 2-(4-Ethoxy-6-fluoro-1-propyl-1H-indol-3-yl)-N,N-dimethylethanamine hydrochloride (Compound 38)

Following the procedure used to prepare compound 22, compound 18 was treated with ethyl iodide to give the product, after formation of the HCl salt using a 4 M solution of HCl in dioxane, in 62% yield as a white solid: mp 154-156° C.; ¹H NMR (300 MHz, DMSO-d₆) δ0.81 (t, 3H, J=7.1), 1.43 (t, 3H, J=6.9), 1.66-1.73 (m, 2H), 2.80 (s, 6H), 3.10-3.15 (m, 2H), 3.23-3.28 (m, 2H), 3.99 (t, 2H, J=6.9), 4.13 (q, 2H, J=6.9), 6.43 (dd, 1H, J=12.1, 1.9), 6.91 (dd, 1H, J=10.2, 1.9), 7.12 (s, 1H), 10.00 (br, 1H). APCI [M+1]: 293.1. Elemental analysis: Calc.: C, 60.76; H, 8.04; N, 8.34; Cl, 10.55. Found: C, 60.86; H, 7.89; N, 8.37; Cl, 10.52 (containing 0.4 equivalent H₂O).

### Example 18

### 2-(6-Fluoro-4-propoxy-1-propyl-1H-indol-3-yl)-N,N-dimethylethanamine hydrochloride (Compound 39)

Following the procedure used to prepare compound 22, compound 18 was treated with propyl iodide to give the product, after formation of the HCl salt using a 4 M solution of HCl in dioxane, in 84% yield as a white solid: mp 170.3-171.4° C.; ¹H NMR (300 MHz, DMSO-d₆) δ0.81 (t, 3H, J=7.1), 1.03 (t, 3H, J=7.1), 1.64-1.76 (m, 2H), 1.78-1.90 (m, 2H), 2.79 (s, 6H), 3.12-3.19 (m, 2H), 3.23-3.30 (m, 2H), 3.99 (t, 2H, J=5.9), 4.03 (t, 2H, J=6.6), 6.43 (dd, 1H, J=12.1, 1.9), 6.91 (dd, 1H, J=10.2, 2.2), 7.12 (s, 1H), 10.1 (br, 1H). APCI [M+1]: 307.2. Elemental analysis: Calc.: C, 63.05; H, 8.23; N, 8.17; Cl, 10.34. Found: C, 62.93; H, 8.23; N, 8.13; Cl, 10.54.

### Example 19

### 2-(5-Fluoro-4-methoxy-1-methyl-1H-indol-3-yl)-N,N-dimethylethanamine hydrochloride (Compound 40)

### Step One: 4-Benzyloxy-3-(2-dimethylaminoethyl)-5-fluoro-1-methyl-1H-indole (Compound 10)

Following the procedure used to prepare compound 4, compound 3 (prepared according to the procedure in Example 22) gave the product in 78% yield. ¹H NMR (300 MHz, CDCl₃) δ 2.14 (s, 6H), 2.50-2.55 (m, 2H), 2.90-2.95 (m, 2H), 3.68 (s, 3H), 5.24 (s, 2H), 6.81 (s, 1H), 6.90 (dd, 1H, J=8.8), 6.98 (dd, 1H, J=12.1, 8.8), 7.31-7.39 (m, 3H), 7.48-7.52 (m, 2H). APCI [M+1]: 327.1.

### Step Two: 3-(2-Dimethylaminoethyl)-5-fluoro-1-methyl-1H-indol-4-ol (Compound 19)

Following the procedure used to prepare compound 13, compound 10 gave the product in 89% yield. ¹H NMR (300 MHz, CDCl₃) δ 2.40 (s, 6H), 2.68-2.71 (m, 2H), 2.89-2.93 (m, 2H), 3.65 (s, 3H), 6.60 (dd, 1H, J=8.8, 3.3), 6.70 (s, 1H), 7.00 (dd, 1H, J=11.5, 8.8). APCI [M+1]: 237.1.

### Step Three: 2-(5-Fluoro-4-methoxy-1-methyl-1H-indol-3-yl)-N,N-dimethylethanamine hydrochloride (Compound 40)

Following the procedure used to prepare compound 22, compound 19 gave the product, after formation of the HCl salt using a 4 M solution of HCl in dioxane, in 32% yield as a white solid: mp 183.6-186.8° C.; ¹H NMR (300 MHz, DMSO-d₆) δ 2.81 (s, 6H), 3.09-3.14 (m, 2H), 3.22-3.27 (m, 2H), 3.71 (s, 3H), 3.99 (d, 3H, J=1.9), 7.05 (dd, 1H, J=12.1, 8.8), 7.12 (dd, 1H, J=8.8, 3.8), 7.22 (s, 1H), 9.97 (br, 1H). APCI [M+1]: 251.1. Elemental analysis: Calc.: C, 58.64; H, 7.03; N, 9.77; Cl, 12.36. Found: C, 58.36; H, 7.00; N, 9.56; Cl, 12.66.

### Example 20

### 2-(1-Ethyl-5-fluoro-4-methoxy-1H-indol-3-yl)-N,N-dimethylethanamine hydrochloride (Compound 41)

### Step One: 4-Benzyloxy-3-(2-dimethylaminoethyl)-1-ethyl-5-fluoro-1H-indole (Compound 11)

Following the procedure used to prepare compound 4, compound 3 (prepared according to the procedure in Example 22) treated with ethyl iodide gave the product in 76% yield. ¹H NMR (300 MHz, CDCl₃) δ 1.42 (t, 3H, J=7.1), 2.13 (s, 6H), 2.50-2.56 (m, 2H), 2.91-2.96 (m, 2H), 4.05 (q, 2H, J=7.1), 5.24 (s, 2H), 6.86 (s, 1H), 6.90 (dd, 1H, J=9.1, 3.8), 6.96 (dd, 1H, J=11.8, 8.8), 7.27-7.40 (m, 3H), 7.50-7.52 (m, 2H). APCI [M+1]: 341.1.

### Step Two: 3-(2-Dimethylaminoethyl)-1-ethyl-5-fluoro-1H-indol-4-ol (Compound 20)

Following the procedure used to prepare compound 13, compound 11 gave the product in 82% yield. ¹H NMR (300 MHz, CDCl₃) δ 1.41 (t, 3H, J=7.1), 2.40 (s, 6H), 2.68-2.71 (m, 2H), 2.90-2.93 (m, 2H), 4.02 (q, 2H, J=7.1), 6.63 (dd, 1H, J=8.8, 3.3), 6.76 (s, 1H), 6.98 (dd, 1H, J=11.2, 8.8). APCI [M+1]: 251.1.

### Step Three: 2-(1-Ethyl-5-fluoro-4-methoxy-1H-indol-3-yl)-N,N-dimethylethanamine hydrochloride (Compound 41)

Following the procedure used to prepare compound 22, compound 20 gave the product, after formation of the HCl salt using a 4 M solution of HCl in dioxane in 32% yield as a white solid: mp 149-150° C.; ¹H NMR (300 MHz, DMSO-d₆) δ 1.31 (t, 3H, J=5.5), 2.81 (s, 6H), 3.10-3.17 (m, 2H), 3.21-3.27 (m, 2H), 3.98 (d, 3H, J=3.1), 4.12 (q, 2H, J=7.4), 7.03 (dd, 1H, J=12.1, 8.8), 7.16 (dd, 1H, J=8.8, 3.6), 7.29 (s, 1H), 10.12 (br, 1H). APCI [M+1]: 265.2. Elemental analysis: Calc.: C, 59.89; H, 7.37; N, 9.31; Cl, 11.79. Found: C, 59.87; H, 7.39; N, 9.16; Cl, 11.94.

### Example 21

### 2-(5-Fluoro-4-methoxy-1-propyl-1H-indol-3-yl)-N,N-dimethylethanamine hydrochloride (Compound 42)

### Step One: 4-Benzyloxy-3-(2-dimethylaminoethyl)-5-fluoro-1-propyl-1H-indole (Compound 12)

Following the procedure used to prepare compound 4, compound 3 (prepared according to the procedure in Example 22) treated with propyl iodide gave the product in 79% yield. ¹H NMR (300 MHz, CDCl₃) δ0.91 (t, 3H, J=7.1), 1.75-1.90 (m, 2H), 2.15 (s, 6H), 2.50-2.55 (m, 2H), 2.90-2.99 (m, 2H), 3.95 (t, 2H, J=6.8), 5.25 (s, 2H), 6.84 (s, 1H), 6.88 (dd, 1H, J=8.8, 3.9), 6.95 (dd, 1H, J=11.8, 9.1), 7.31-7.4 (m, 3H), 7.49-7.52 (m, 2H). APCI [M+1]: 355.1.

### Step Two: 3-(2-Dimethylaminoethyl)-5-fluoro-1-propyl-1H-indol-4-ol (Compound 21)

Following the procedure used to prepare compound 13, compound 12 gave the product in 91% yield. ¹H NMR (300 MHz, CDCl₃) δ 0.91 (t, 3H, J=7.4), 1.75-1.87 (m, 2H), 2.4 (s, 6H), 2.68-2.71 (m, 2H), 2.90-2.93 (m, 2H), 3.92 (t, 2H, J=7.1), 6.62 (dd, 1H, J=8.8, 3.3), 6.74 (s, 1H), 6.97 (dd, 1H, J=11.2, 8.8). APCI [M+1]: 265.1.

### Step Three: 2-(5-Fluoro-4-methoxy-1-propyl-1H-indol-3-yl)-N,N-dimethylethanamine hydrochloride (Compound 42)

Following the procedure used to prepare compound 22, compound 21 gave the product, after formation of the HCl salt using a 4 M solution of HCl in dioxane, in 39% yield as a pink solid: mp 111-113° C.; ¹H NMR (300 MHz, DMSO-d₆) δ0.82 (t, 3H, J=7.4), 1.66-1.78 (m, 2H), 2.81 (s, 6H), 3.11-3.15 (m, 2H), 3.22-3.27 (m, 2H), 4.00 (d, 3H, J=1.9), 4.05 (t, 2H, J=6.9), 7.02 (dd, 1H, J=12.1, 9.1), 7.16 (dd, 1H, J=9.1, 3.6), 7.27 (s, 1H), 10.30 (br, 1H). APCI [M+1]: 279.1. Elemental analysis: Calc.: C, 61.04; H, 7.68; N, 8.90; Cl, 11.26. Found: C, 61.05; H, 7.67; N, 8.73; Cl, 11.17.

### Example 22

### 2-(4-Benzyloxy-5-fluoro-1H-indol-3-yl)-N,N-dimethylethanamine (Compound 3)

### Step One: Methyl 4-benzyloxy-5-fluoro-1H-indole-2-carboxylate (46)

In a pre-dried 3-neck roundbottom flask was added anhydrous MeOH (100 mL), followed by Na metal (5.52 g, 0.24 mol) portion wisely at 0° C. The resulting solution was cooled in a dry ice/acetone bath to −20° C. A solution of 44 (prepared according to U.S. Pat. No. 5,330,992) (18.4 g, 0.08 mol) and methyl azidoacetate (27.6 g, 0.24 mol) in dry MeOH (50 mL) was added dropwise over 60 min. After stirring for 1 h, the reaction was warmed to room temperature and stirring continued for 1.5 h. The heterogeneous mixture was then poured onto ice, and the precipitate was collected by filtration. The yellow solid (45) was immediately dissolved in p-xylenes (400 mL) and the solution was washed with brine, followed by drying over Na₂SO₄. After filtration the resulting solution was heated at reflux until TLC indicated the reaction was complete (about 1 h). The solvent was distilled under reduced pressure to precipitate the product as white crystals (6.73 g). An additional 1.36 g of product was obtained by chromatography of the residue on silica gel, eluting with EtOAc/hexanes (overall yield 35%). ¹H NMR (300 MHz, CDCl₃) δ 3.94 (s, 3H), 5.33 (s, 2H), 6.98-7.03 (m, 1H), 7.10 (dd, 1H, J=11.8, 8.8), 7.29-7.41 (m, 4H), 7.48-7.50 (m, 2H), 8.81 (br, 1H). APCI [M+1]: 300.2.

### Step Two: 4-(Benzyloxy)-5-fluoro-1H-indole-2-carboxylic acid (47)

Compound 46 was added to a solution of aqueous 2 N NaOH. The suspension was stirred at 80-90° C. until the reaction mixture became clear and was then held at reflux for 1-2 h. The solution was cooled and acidified with aqueous 3 N HCl, then the resulting precipitate was collected by filtration, washed with water, and dried under vacuum over P₂O₅ to provide the product in 99% yield. ¹H NMR (CDCl₃, 300 MHz) δ 5.31 (s, 2H), 7.06-7.18 (m, 3H), 7.30-7.42 (m, 3H), 7.47-7.50 (m, 2H), 11.93 (bs, 1H).

### Step Three: 4-Benzyloxy-5-fluoro-1H-indole (48)

Compound 47 (6.28 g, 0.022 mol), copper powder (7.05 g, 0.11 mol) and 2-phenylpyridine (50 mL) were heated at reflux (230-240° C.) under a stream of nitrogen for 10 min, by which time TLC analysis showed complete reaction. The reaction mixture was cooled, filtered through Celite, and the filter cake was washed with EtOAc. The filtrate and EtOAc washings were combined, diluted with water, and extracted three times with EtOAc. The organic extract was washed with 1 N HCl, H₂O and brine, dried with NaSO₄, and concentrated. The resulting residue was purified by column chromatography on silica gel eluting with EtOAc/hexanes to give the product as a greenish solid in 62% yield, 3.32 g. ¹H NMR (300 MHz, CDCl₃) δ5.31 (s, 2H), 6.62-6.64 (m, 1H), 6.93-7.03 (m, 2H), 7.15 (dd, 1H, J=3.0, 2.5), 7.31-7.40 (m, 3H), 7.49-7.52 (m, 2H), 8.12 (br, 1H). APCI [M+1]: 242.1.

### Step Four: 2-(4-Benzyloxy-5-fluoro-1H-indol-3-yl)-N,N-dimethyl-2-oxoacetamide (49)

A solution of oxalyl chloride (0.78 mL, 8.963 mmol) in anhydrous ether (20 mL) was added dropwise over 20 min to a 0° C. solution of 48 (1.44 g, 5.975 mmol) in anhydrous ether (20 mL). The reaction mixture was stirred at room temperature for 5 h, cooled to −20° C., and treated with a stream of dimethyl amine gas. The reaction was diluted with EtOAc, washed with water and brine, and dried over Na₂SO₄. After concentration, the resulting residue was recrystallized from EtOAc to provide the product as white crystals in 87% yield, 1.58 g. ¹H NMR (300 MHz, CDCl₃) δ 2.92 (s, 3H), 2.94 (s, 3H), 5.17 (s, 2H), 6.91-7.00 (m, 2H), 7.29-7.37 (m, 3H), 7.58-7.61 (m, 2H), 7.70-7.72 (m, 1H), 10.02 (br, 1H). APCI [M+1]: 341.2.

### Step Five: 2-(4-Benzyloxy-5-fluoro-1H-indol-3-yl)-N,N-dimethylethanamine (3)

A solution of 49 (2.26 g, 6.647 mmol) in dry 1,4-dioxane (30 mL) was added dropwise to a slurry of LiAlH₄ (2.52 g, 66.47 mmol) in dry 1,4-dioxane (40 mL) at reflux. The mixture was held at reflux for 1 h. The mixture was then cooled, quenched with ice-water (mixed with NaOH), filtered through Celite, and the filter cake was washed with EtOAc. The filtrate and EtOAc washings were combined and extracted three times with EtOAc. The organic layer was washed with 1 N NaOH and brine, dried over Na₂SO₄ and concentrated. The resulting residue was purified by column chromatography on silica gel with CH₂Cl₂ (100%) to 5% NH₄OH in CH₂Cl₂ as eluent to give the product as a brown oil in 80% yield, 1.67 g. ¹H NMR (300 MHz, CDCl₃) δ 2.14 (s, 6H). 2.52-2.57 (m, 2H), 2.91-2.97 (m, 2H), 5.25 (s, 2H), 6.92-6.98 (m, 3H), 7.31-7.40 (m, 3H), 7.49-7.52 (m, 2H), 7.97 (br, 1H). APCI [M+1]: 313.1.

### Example 23

### FIG. 3

### 4-Benzyloxy-3-(2-dimethylaminoethyl)-7-fluoro-1-methyl-1H-indole (Compound 50)

At 0° C., NaH (2 eq) was added to a solution of 1 eq of compound 1 (prepared according to J. Med. Chem. 2000, 43, 4701) in DMF. After 30 min, a solution of 0.1 M methyl iodide in DMF was added slowly over 30 min, and then the reaction mixture was allowed to warm to 25° C. and stirred at that temperature for 1 h. After standard workup, the brown oil residue was purified by column chromatography, eluting with CH₂Cl₂/2% NH₄OH. A white solid was obtained in 84% yield, mp 60-62° C. ¹H NMR (CDCl₃, 300 MHz) δ 2.13 (s, 6H), 2.50-2.56 (m, 2H), 2.95-3.01 (m, 2H), 3.89 (d, 3H, J=1.9), 5.13 (s, 2H), 6.29 (dd, 1H, J=8.5, 2.8), 6.67 (dd, 1H, J=8.5, 12.1), 6.69 (s, 1H), 7.29-7.40 (m, 3H), 7.45-7.48 (m, 2H). APCI [M+1]: 327.2. Elemental analysis C₂₀H₂₃FN₂O+0.1H₂O: Calc.: C, 73.19; H, 7.12; N, 8.54. Found: C, 73.17; H, 7.12; N, 8.51.

### Example 24

### FIG. 3

### 4-Benzyloxy-3-(2-dimethylaminoethyl)-6-fluoro-1-methyl-1H-indole (Compound 51)

Following the procedure used to prepare compound 50, compound 2 (prepared according to J. Med. Chem. 2000, 43, 4701) gave the product in 76% yield, mp 79-80° C. ¹H NMR (CDCl₃, 300 MHz) δ2.13 (s, 6H), 2.50-2.57 (m, 2H), 2.95-3.01 (m, 2H), 3.63 (s, 3H), 5.14 (s, 2H), 6.34 (dd, 1H, J=11.8, 1.9), 6.56 (dd, 1H, J=9.5, 2.2), 6.71 (s, 1H), 7.31-7.41 (m, 3H), 7.45-7.52 (m, 2H). APCI [M+1]: 327.1. Elemental analysis: Calc.: C, 73.59; H, 7.10; N, 8.58. Found: C, 73.20; H, 7.16; N, 8.46.

### Example 25

### FIG. 3

### 4-Benzyloxy-3-(2-dimethylaminoethyl)-5-fluoro-1-methyl-1H-indole hydrochloride (Compound 52)

Following the procedure used to prepare compound 50, compound 3 (prepared according to the procedure in Example 22) gave the product in 78% yield as an off-white solid, after formation of the HCl salt, mp 139-141° C. ¹H NMR (DMSO-d₆, 300 MHz) δ2.58 (s, 6H), 2.99-3.05 (m, 2H), 3.13-3.20 (m, 2H), 3.71 (s, 3H), 5.24 (s, 2H), 7.00-7.15 (m, 2H), 7.20 (s, 1H), 7.31-7.39 (m, 3H), 7.48-7.52 (m, 2H), 9.95 (bs, 1H). APCI [M+1]: 327.1. Elemental analysis C₂₀H₂₄ClFN₂O+0.8H₂O: Calc.: C, 63.67; H, 6.84; N, 7.43; Cl, 9.40. Found: C, 63.70; H, 6.82; N, 7.40; Cl, 9.40.

### Example 26

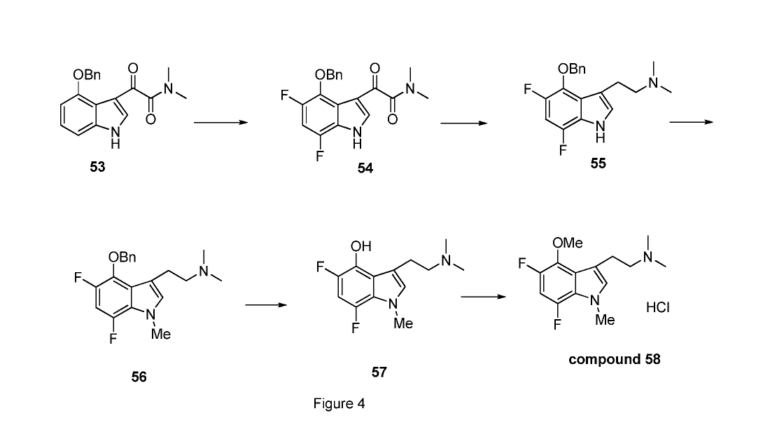

### FIG. 4

### 2-(5,7-Difluoro-4-methoxy-1-methyl-1H-indol-3-yl)-N,N-dimethylethanamine (Compound 58)

### Step One: 2-(4-Benzyloxy-5,7-difluoro-1H-indol-3-yl)-N,N-dimethyl-2-oxoacetamide (54)

Compound 53, prepared according to Helv. Chim. Acta, 1959, 42, 1557 (24.2 g, 75.1 mmol) was dissolved in acetonitrile (˜2 L) at room temperature. To this stirred solution Selectfluor (53 g, 150 mmol) was added portionwise over 30 min and the reaction was monitored by MS. After 4-5 h the reaction was complete. The reaction mixture was concentrated and the crude mixture was dissolved in acetone, solids were filtered off, and the brown filtrate was concentrated. The crude mixture was adsorbed on silica gel and purified by column chromatography (EtOAc) to give compound 54 as a white solid; ¹H NMR (CDCl₃, 300 MHz) δ 2.93 (s, 3H), 2.94 (s, 3H), 5.10 (s, 2H), 6.80 (dd, 1H, J=10.7, 9.6), 7.26-7.37 (m, 3H), 7.56 (m, 2H), 7.77 (s, 1H), 10.86 (bs, 1H). APCI [M+1]: 359.2.

### Step Two: 2-(4-Benzyloxy-5,7-difluoro-1H-indol-3-yl)-N,N-dimethylethanamine (55)

To a refluxing suspension of LiAlH₄ in 1,4-dioxane (50 mL) was added a solution of compound 54 (950 mg, 2.67 mmol) in 1,4-dioxane dropwise and the reaction mixture was stirred at reflux for 3 h, then cooled to 0° C. and quenched by slowly adding water, 15% NaOH, and water. Solid precipitate was filtered off and the filtrate was extracted with CH₂Cl₂ and washed with water, dried, and concentrated. The crude material was purified by column chromatography (10% NH₄OH in CH₂Cl₂) to give the product compound 55 (410 mg, 47%) as a white solid. ¹H NMR (CDCl₃, 300 MHz) δ2.16 (s, 6H), 2.52-2.58 (m, 2H), 2.89-2.96 (m, 2H), 5.18 (s, 2H), 6.72 (t, 1H, J=11.0), 6.97 (s, 1H), 7.32-7.41 (m, 3H), 7.46-7.52 (m, 2H), 8.28 (bs, 1H). APCI [M+1]: 331.1.

### Step Three: 2-(4-(Benzyloxy)-5,7-difluoro-1-methyl-1H-indol-3-yl)-N,N-dimethylethanamine (Compound 56)

To a solution of compound 55 (410 mg, 1.24 mmol) in DMF at 0° C. was added NaH (60 mg, 2.5 mmol) and the reaction mixture was stirred for 10 min, then 0.1 M methyl iodide (12.4 ml, 1.24 mmol) was added dropwise and the reaction was monitored by MS. After completion, the reaction was quenched by adding water and EtOAc and washed with water, dried, and concentrated. The crude product was purified by column chromatography (10% NH₄OH in CH₂Cl₂) to give compound 56 (267 mg, 62%). ¹H NMR (CDCl₃, 300 MHz) δ 2.13 (s, 6H), 2.48-2.53 (m, 2H), 2.86-2.92 (m, 2H), 3.86 (d, 3H, J=1.9), 5.16 (s, 2H), 6.69 (t, 1H, J=11.7), 6.75 (s, 1H), 7.31-7.40 (m, 3H), 7.46-7.50 (m, 2H). APCI [M+1]: 345.2.

### Step Four: 3-(2-Dimethylaminoethyl)-5,7-difluoro-1-methyl-1H-indol-4-ol (Compound 57)

10% Pd(OH)₂/C (20 mg) was added to a solution of compound 56 (260 mg, 0.78 mmol) in MeOH (30 mL) and the flask stirred under an H₂ atmosphere for 45 min. After completion of the reaction it was filtered through Celite and the solvent was removed. The crude mixture was purified by column chromatography (10% NH₄OH in CH₂Cl₂) to obtain compound 57 (160 mg, 81%) as an oil. ¹H NMR (CDCl₃, 300 MHz) δ2.37 (s, 6H), 2.65-2.68 (m, 2H), 2.86-2.89 (m, 2H), 3.83 (d, 3H, J=2.2), 6.63 (s, 1H), 6.72 (t, 1H, J=11.3). APCI [M+1]: 255.1.

### Step Five: 2-(5,7-Difluoro-4-methoxy-1-methyl-1H-indol-3-yl)-N,N-dimethylethanamine hydrochloride (Compound 58)

To a solution of compound 57 (155 mg, 0.61 mmol) in DMF at 0° C. was added NaH (30 mg, 1.2 mmol) and the reaction mixture was stirred for 10 min. To this reaction mixture 0.1 M methyl iodide (0.61 mmol, 1.0 equiv) was added dropwise, and the reaction was monitored by MS. After completion, the reaction was quenched by adding water and extracted with EtOAc, dried, and concentrated. The crude product was purified by column chromatography (10% NH₄OH in CH₂Cl₂) to give the product compound 58 (60 mg, 37%) as a brown solid after formation of the HCl salt, mp 185-189° C.; ¹H NMR (DMSO-d₆, 300 MHz) δ 2.81 (s, 6H), 3.10-3.16 (m, 2H), 3.20-3.25 (m, 2H), 3.86 (d, 3H, J=2.2), 3.94 (d, 3H, J=1.7), 7.05 (t, 1H, J=12.0), 7.26 (s, 1H). APCI [M+1]: 269.1. Elemental analysis: Calc.: C, 55.17; H, 6.28; N, 9.19; Cl, 11.63. Found: C, 55.31; H, 6.33; N, 9.02; Cl, 11.82.

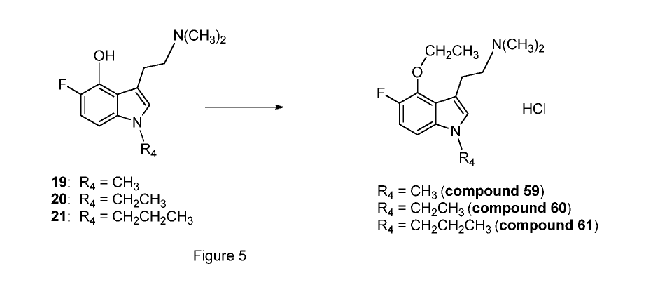

### General Procedure for Preparation of 59, 60, and 61 (FIG. 5):

To a solution of 19, 20 or 21 in DMF at 0° C. was added NaH (2 eq) and the reaction mixture was stirred for 10 min. Then 0.1 M alkyl iodide (1.05 eq) was added dropwise and the reaction was monitored by MS. After completion, the reaction was quenched by adding water and EtOAc and washed with water, dried, and concentrated. The crude product was purified by column chromatography and converted to the HCl salt.

### Example 27

### 2-(4-Ethoxy-5-fluoro-1-methyl-1H-indol-3-yl)-N,N-dimethylethanamine hydrochloride (Compound 59)

Starting with Compound 19, Compound 59 was obtained as a brown solid, mp 163-165° C. ¹H NMR (DMSO-d₆, 300 MHz) δ 1.37 (t, 3H, J=7.1), 2.81 (s, 6H), 3.13-3.21 (m, 2H), 3.24-3.30 (m, 2H), 3.71 (s, 3H), 4.22 (dq, 2H, J=7.1, 1.4), 6.99-7.13 (m, 2H), 7.23 (s, 1H), 10.34 (bs, 1H). APCI [M+1]: 251.1. Elemental analysis: Calc.: C, 59.89; H, 7.37; N, 9.31; Cl, 11.79. Found: C, 59.91; H, 7.47; N, 9.25; Cl, 11.90

### Example 28

### 2-(4-Ethoxy-1-ethyl-5-fluoro-1H-indol-3-yl)-N,N-dimethylethanamine hydrochloride (Compound 60)

Starting with Compound 20, Compound 60 was obtained as a light brown solid, mp 130-132° C. ¹H NMR (CDCl₃, 300 MHz) δ 1.42 (t, 3H, J=7.4), 1.43 (dt, 3H, J=7.1, 0.8), 2.81 (s, 3H), 2.82 (s, 3H), 3.27-3.35 (m, 2H), 3.37-3.44 (m, 2H), 4.06 (q, 2H, J=7.1), 4.33 (dq, 2H, J=6.9, 1.4), 6.88-7.00 (m, 2H), 7.07 (s, 1H). APCI [M+1]: 279.2. Elemental analysis C₁₆H₂₃FN₂O+1.1HCl: Calc.: C, 59.66; H, 7.57; N, 8.70; Cl, 11.26. Found: C, 59.31; H, 7.53; N, 8.73; Cl, 11.44.

### Example 29

### 2-(4-Ethoxy-5-fluoro-1-propyl-1H-indol-3-yl)-N,N-dimethylethanamine hydrochloride (Compound 61)

Starting with Compound 21, Compound 61 was obtained as a white solid, mp 141-143° C. ¹H NMR (CDCl₃, 300 MHz) δ 0.90 (t, 3H, J=7.4), 1.42 (t, 3H, J=6.9), 1.74-1.87 (m, 2H), 2.81 (s, 6H), 3.27-3.45 (m, 4H), 3.96 (t, 2H, J=7.1), 4.33 (dq, 2H, J=6.9, 1.4), 6.84-7.00 (m, 2H), 7.04 (s, 1H). APCI [M+1]: 293.1. Elemental analysis C₁₇H₂₆ClFN₂O+0.5H₂O: Calc.: C, 60.43; H, 8.05; N, 8.29; Cl, 10.49. Found: C, 60.36; H, 8.07; N, 8.43; Cl, 10.51

### Example 30

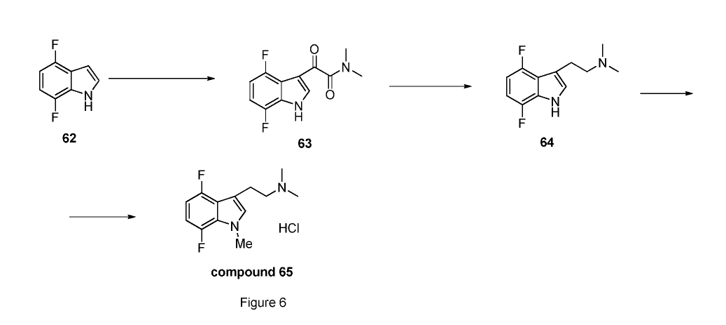

### FIG. 6

### 2-(4,7-Difluoro-1-methyl-1H-indol-3-yl)-N,N-dimethylethanamine hydrochloride (Compound 65)

### Step One: 2-(4,7-Difluoro-1H-indol-3-yl)-N,N-dimethyl-2-oxoacetamide (63)

A solution of oxalyl chloride (1.37 mL, 15.67 mmol) in anhydrous ether (6 mL) was added dropwise over 10 min to a 0° C. solution of compound 62 (1.60 g, 10.45 mmol) in anhydrous ether (10 mL). The reaction solution was stirred at room temperature overnight (it became cloudy and some yellowish precipitate was formed after stirring for 3 h). The reaction mixture was diluted with anhydrous ether (15 mL) and cooled to 0° C. and dimethylamine gas was directly bubbled into the reaction flask until the pH of the reaction mixture had turned basic. The resulting mixture was stirred overnight at room temperature. The reaction mixture was diluted with EtOAc and washed with water. The combined aqueous layer was extracted with CH₂Cl₂ until there is no product in the aqueous layer (5×). The combined organic layer was dried and concentrated to give compound 63 as a white solid (1.95 g, 74%). ¹H NMR (CDCl₃, 300 MHz) δ 3.08 (s, 3H), 3.10 (s, 3H), 6.96-6.81 (m, 2H), 8.05 (d, 1H, J=3.0), 9.45 (bs, 1H). APCI [M+1]: 253.1.

### Step Two: 2-(4,7-Difluoro-1H-indol-3-yl)-N,N-dimethylethanamine (Compound 64)

To a refluxing suspension of LiAlH₄ (3.16 g, 83.33 mmol) in anhydrous THF (60 mL) was added a solution of compound 63 (2.10 g, 8.33 mmol) in anhydrous THF (60 mL) dropwise and the reaction mixture was stirred at reflux for 3 h. The reaction was cooled to 0° C. and quenched by slowly by adding 3.2 mL water, 3.2 mL of 15% NaOH solution and then 10 mL of water. The resulting slurry was stirred for 20 min. Solid precipitate was filtered off and the filtrate was extracted with CH₂Cl₂ and washed with water, dried and concentrated. The crude material was purified by column chromatography (5% NH₄OH in CH₂Cl₂) to give the product compound 64 (1.49 g, 80%) as a colorless oil. ¹H NMR (CDCl₃, 300 MHz) δ 2.33 (s, 6H), 2.62-2.68 (m, 2H), 2.96-3.02 (m, 2H), 6.52-6.71 (m, 2H), 6.92 (d, 1H, J=1.9), 8.81 (bs, 1H); APCI [M+1]: 225.1.

### Step Three: 2-(4,7-Difluoro-1-methyl-1H-indol-3-yl)-N,N-dimethylethanamine hydrochloride (Compound 65)

To a solution of compound 64 in DMF at 0° C. was added NaH (2 eq) and the reaction mixture was stirred for 10 min and then 0.1 M methyl iodide (1.05 eq) was added dropwise and the reaction was monitored by MS. After completion, the reaction was quenched by adding water and EtOAc, and washed with water, dried, and concentrated. The crude product was purified by column chromatography to give a brown oil, which was converted into the HCl salt, compound 65, as a white solid, mp 209-210° C. ¹H NMR (DMSO-d₆, 300 MHz) δ 2.80 (s, 6H), 3.10-3.18 (m, 2H), 3.22-3.29 (m, 2H), 3.91 (d, 3H, J=1.9), 6.70-6.78 (m, 1H), 6.87-6.95 (m, 1H), 7.29 (s, 1H), 10.32 (bs, 1H); APCI [M+1]: 239.1. Elemental analysis: Calc.: C, 56.83; H, 6.24; N, 10.20; Cl, 12.90. Found: C, 56.68; H, 6.19; N, 10.12; Cl, 13.08.

### Example 31

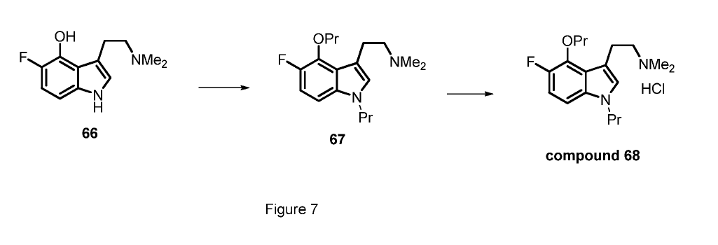

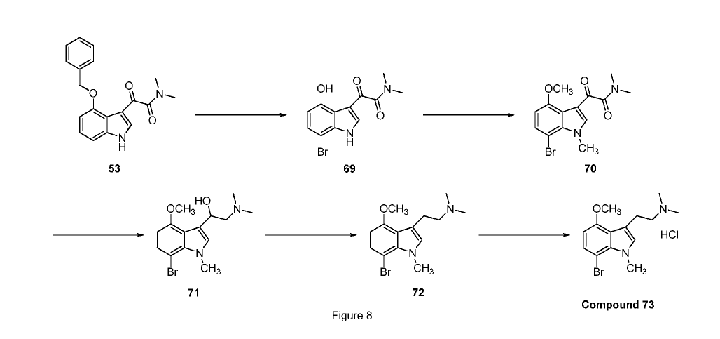

### FIG. 7

### 2-(5-Fluoro-4-propoxy-1-propyl-1H-indol-3-yl)-N,N-dimethylethanamine hydrochloride (Compound 68)

### Step One: 2-(5-Fluoro-4-propoxy-1-propyl-1H-indol-3-yl)-N,N-dimethylethanamine (67)

At 0° C., 1.17 g of crude material of 3-(2-dimethylaminoethyl)-5-fluoro-1H-indol-4-ol (66), which was obtained as byproduct during the reduction of compound 49 to compound 3, in 50 mL DMF was treated with NaH (300 mg, 7.5 mmol). After stirring at this temperature for 30 min, a solution of 0.1 M propyl iodide in DMF (28 mL) was added over 45 min, and the reaction mixture was stirred for additional 15 min. TLC and MS showed the reaction was complete and it was quenched by addition of brine. The mixture was extracted with EtOAc, and the combined organic layer was washed with water and then brine. After being dried over anhydrous Na₂SO₄, the solution was concentrated to give a dark brown oil, which was purified by flash column chromatography (SiO₂: CH₂Cl₂/NH₄OH 0% to 5%) to afford impure compound 67 as a brown oil (1.04 g).

A mixture of Pd(OH)₂/C (200 mg) with the above batch of compound 67 (containing some 12) in 100 mL MeOH was hydrogenated for 10 min by which time MS showed 12 had been consumed. The reaction mixture was filtered through Celite and washed with MeOH. The solution was concentrated to obtain a dark oil, which was purified by flash column chromatography (SiO₂: CH₂Cl₂/NH₄OH 0% to 5%) to afford the mixture of products 67 and 21 as a brown oil (503 mg). The mixture was purified by recrystallization from hexanes and ether. The filtrate was purified by reverse phase column chromatography (C18: water/MeOH 0.1% TFA) to give the pure product 67 as a brown oil (446 mg). ¹H NMR (CDCl₃, 300 MHz) δ 0.91 (t, 3H, J=7.6), 1.04 (t, 3H, J=7.4), 1.74-1.93 (m, 4H), 2.31 (s, 6H), 2.58-2.64 (m, 2H), 2.98-3.06 (m, 2H), 3.94 (t, 2H, J=7.0), 4.17 (td, 2H, J=6.9, 1.7), 6.82-6.95 (m, 3H). APCI [M+1]: 307.2.

### Step Two: 2-(5-Fluoro-4-propoxy-1-propyl-1H-indol-3-yl)-N,N-dimethylethanamine hydrochloride (Compound 68)

Compound 67 (446 mg, 1.45 mmol) was dissolved in 100 mL ether and treated with 2.0 M HCl in Et₂O (1.50 mL). After stirring at room temperature for 30 min, the suspension was filtered and washed with ether to give the compound 68 as a white solid (395 mg, 79%), mp 142-145° C. ¹H NMR (CDCl₃, 300 MHz) δ 0.82 (t, 3H, J=7.2), 0.99 (t, 3H, J=7.4), 1.66-1.85 (m, 4H), 2.81 (s, 6H), 3.12-3.20 (m, 2H), 3.27-3.35 (m, 2H), 4.04 (t, 2H, J=6.7), 4.11 (t, 2H, J=6.8), 7.01 (dd, 1H, J=12.1, 9.1), 7.15 (dd, 1H, J=8.9, 2.6), 7.28 (s, 1H). APCI [M+1]: 307.2. Elemental analysis: Calc.: C, 63.05; H, 8.23; N, 8.17; Cl, 10.34. Found: C, 62.91; H, 8.20; N, 8.07; Cl, 10.22.

### Example 32

### FIG. 8

### 2-(7-Bromo-4-methoxy-1-methyl-1H-indol-3-yl)-N,N-dimethylethanamine hydrochloride (Compound 73)

### Step One: 2-(7-Bromo-4-hydroxy-1H-indol-3-yl)-N,N-dimethyl-2-oxoacetamide (Compound 69)

To a solution of compound 53, prepared according to Helv. Chim. Acta, 1959, 42, 1557 (3.22 g, 10 mmol) in CH₂Cl₂ (90 mL) and ether (75 mL) was added pyridinium hydrobromide perbromide and the reaction mixture was stirred overnight at 25° C. Solvent was removed under reduced pressure and the crude material was purified by column chromatography (elution with ethyl acetate/hexanes) to give compound 69 (2.04 g, 65%) as a yellow solid. ¹H NMR (CD₃OD, 300 MHz) δ 3.07 (s, 3H), 3.11 (s, 3H), 6.57 (d, 1H, J=8.5), 7.30 (d, 1H, J=8.5), 7.98 (s, 1H). APCI [M+1]: 311, 313.

### Step Two: 2-(7-Bromo-4-methoxy-1-methyl-1H-indol-3-yl)-N,N-dimethyl-2-oxoacetamide (Compound 70)

To a solution of compound 69 (1.28 g, 4.12 mmol) in DMF at 0° C. was added NaH (660 mg, 16.5 mmol) and the reaction mixture was stirred for 10 min. To this reaction mixture, methyl iodide (3.5 g, 24.7 mmol) was added slowly and the reaction was monitored by MS. After 2 h, the reaction was quenched by adding water and EtOAc and washed with water, dried, and concentrated. The crude product was purified by column chromatography (1% NH₄OH/1% MeOH/EtOAc) to obtain compound 70 (1.30 g, 92%) as a white solid. ¹H NMR (CDCl₃, 300 MHz) δ 3.07 (s, 3H), 3.09 (s, 3H), 3.88 (s, 3H), 4.19 (s, 3H), 6.51 (d, 1H, J=8.5), 7.32 (d, 1H, J=8.5), 7.79 (s, 1H). APCI [M+1]: 339, 341.

### Step Three: 1-(7-Bromo-4-methoxy-1-methyl-1H-indol-3-yl)-2-(dimethylamino)ethanol (Compound 71)

To a slurry of LiAlH₄ (419 mg, 11.03 mmol) in anhydrous THF at 0° C. under a nitrogen atmosphere was added AlCl₃ (488 mg, 3.67 mmol) and the reaction mixture was stirred for 10 min. To this slurry was then added compound 70 (500 mg, 1.47 mmol) and stirring continued for 30 min by which time MS showed no starting material. The reaction was quenched by adding water, extracted by ether, dried, and concentrated. The crude material was purified by column chromatography to give compound 71 (480 mg, 90%) as a white solid. ¹H NMR (CDCl₃, 300 MHz) δ2.42 (s, 6H), 2.58 (dd, 1H, J=12.4, 9.6), 2.71 (dd, 1H, J=12.4, 3.3), 3.89 (s, 3H), 4.08 (s, 3H), 5.28 (dd, 1H, J=9.2, 3.0), 6.33 (d, 1H, J=8.5), 6.97 (s, 1H), 7.20 (d, 1H, J=8.3). APCI [M+1]: 309, 311, 327, 329.

### Step Four: 2-(7-Bromo-4-methoxy-1-methyl-1H-indol-3-yl)-N,N-dimethylethanamine (Compound 72)

To a solution of compound 71 (430 mg, 1.31 mmol) in anhydrous CH₂Cl₂ (20 mL) at 0° C. was added Et₃SiH (1.52 g, 13.1 mmol) and the reaction mixture was stirred for 10 min. To this mixture was added CF₃CO₂H (1.2 g, 10.5 mmol) and stirring continued for 30 min. The reaction was quenched by adding sodium bicarbonate and it was extracted with CH₂Cl₂ and washed by water and brine. The organic layer was dried over sodium sulfate and concentrated. The crude material was purified by column chromatography (2% NH₄OH in CH₂Cl₂) to get compound 72 (76 mg, 12%) as an off-white solid. ¹H NMR (CDCl₃, 300 MHz) δ2.31 (s, 6H), 2.51-2.57 (m, 2H), 2.95-3.02 (m, 2H), 3.87 (s, 3H), 4.06 (s, 3H), 6.30 (d, 1H, J=8.3), 6.67 (s, 1H), 7.17 (d, 1H, J=8.5). APCI [M+1]: 311.2, 313.2.

### Step Five: 2-(7-Bromo-4-methoxy-1-methyl-1H-indol-3-yl)-N,N-dimethylethanamine hydrochloride (Compound 73)

Compound 72 (59 mg, 0.19 mmol) was dissolved in 20 mL ether and treated with 2.0 M HCl in Et₂O (4 mL) dropwise. After stirring at room temperature for 30 min, the suspension was filtered and washed with ether to give compound 73 as an off-white solid (64 mg, 97%), mp 235-236° C. ¹H NMR (DMSO-d₆, 300 MHz) δ 2.80 (s, 6H), 3.09-3.24 (m, 4H), 3.87 (s, 3H), 4.03 (s, 3H), 6.47 (d, 1H, J=8.5), 7.14 (s, 1H), 7.22 (d, 1H, J=8.3), 10.13 (bs, 1H). APCI [M+1]: 311.2, 313.2. Elemental analysis C₁₄H₁₉BrN₂O+1.07 HCl: Calc.: C, 48.01; H, 5.78; N, 8.00; Br, 22.81; Cl, 10.83. Found: C, 47.92; H, 5.78; N, 7.85; Br, 23.03; Cl, 10.87.

### Example 33

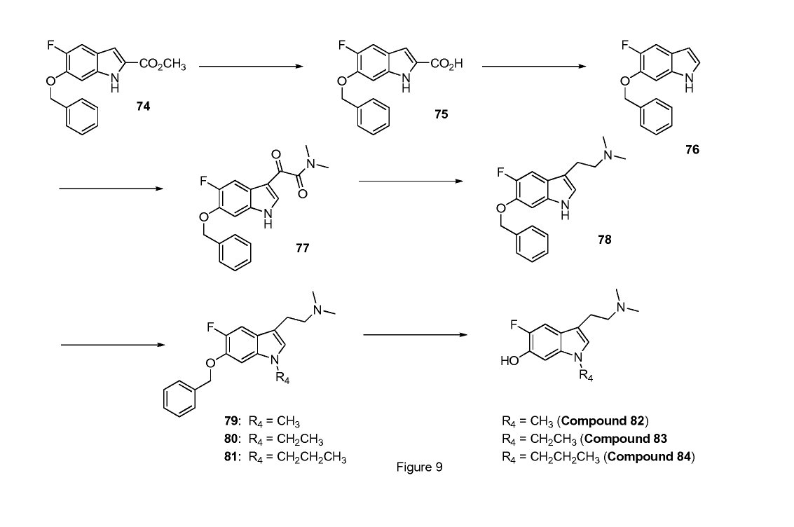

### FIG. 9

### 3-[2-(Dimethylamino-ethyl)]-5-fluoro-1-methyl-1H-indol-6-ol (Compound 82)

### Step One: 6-Benzyloxy-5-fluoro-1H-indole-2-carboxylic acid (Compound 75)

A suspension of compound 74, prepared according to WO2005/123716, (3.09 g, 10.3 mmol) in 150 mL of 2 M NaOH solution was heated at reflux for 4 h. The clear solution was cooled to 0° C., and acidified by 3 M HCl to pH-1, as white precipitate appeared. The suspension was filtered and washed with water, and then dried under vacuum (75° C.) overnight. Compound 75 was obtained as a white solid (2.89 g, 98%). ¹H NMR (DMSO-d₆, 300 MHz) δ 5.19 (s, 2H), 7.01 (d, 1H, J=1.6), 7.09 (d, 1H, J=8.0), 7.32-7.52 (m, 6H), 11.70 (bs, 1H). APCI [M−1]: 284.

### Step Two: 6-Benzyloxy-5-fluoro-1H-indole (Compound 76)

A mixture of compound 75 (2.58 g, 9.07 mmol) and copper (2.97 g, 45.1 mmol) in 80 mL of 1-methylpyrrolidin-2-one was heated at reflux overnight under nitrogen. After cooling to room temperature, the mixture was filtered through Celite and washed with EtOAc. The filtrate was partitioned between water and EtOAc (2×150 mL). The combined organic phase was washed with water and brine, and then dried over anhydrous Na₂SO₄. The black residue was purified by column chromatography (hexanes/EtOAc 2% to 12%) to give compound 76 (1.31 g, 60%) as a white solid. ¹H NMR (CDCl₃, 300 MHz) δ 5.16 (s, 2H), 6.94-6.98 (m, 1H), 6.96 (d, 1H, J=7.1), 7.10-7.14 (m, 1H), 7.29-7.42 (m, 4H), 7.44-7.50 (m, 2H), 8.01 (bs, 1H). APCI [M−1]: 240.

### Step Three: 2-(6-Benzyloxy-5-fluoro-1H-indol-3-yl)-N,N-dimethyl-2-oxoacetamide (Compound 77)

Following the procedure used to prepare compound 49, compound 76 gave compound 77 in 82% yield as a white solid. ¹H NMR (CDCl₃, 300 MHz) δ 3.06 (s, 3H), 3.08 (s, 3H), 5.14 (s, 2H), 6.94 (d, 1H, J=6.9), 7.32-7.47 (m, 5H), 7.82 (d, 1H, J=3.0), 8.05 (d, 1H, J=11.3), 8.96 (bs, 1H). APCI [M+1]: 386.2.

### Step Four: 2-(6-Benzyloxy-5-fluoro-1H-indol-3-yl)-N,N-dimethylethanamine (Compound 78)

Following the procedure used to prepare compound 3, compound 77 gave compound 78 in 77% yield as a brown oil. ¹H NMR (CDCl₃, 300 MHz) δ 2.32 (s, 6H), 2.56-2.62 (m, 2H), 2.82-2.88 (m, 2H), 6.88-6.94 (m, 2H), 7.28-7.40 (m, 4H), 7.44-7.50 (m, 2H), 7.88 (bs, 1H). APCI [M+1]: 313.2 .

### Step Five: 2-(6-Benzyloxy-5-fluoro-1-methyl-1H-indol-3-yl)-N,N-dimethylethanamine (Compound 79)

Following the procedure used to prepare compound 4, compound 78 gave the product compound 79 in 73% yield as a yellow oil. ¹H NMR (CDCl₃, 300 MHz) δ 2.31 (s, 6H), 2.53-2.59 (m, 2H), 2.80-2.88 (m, 2H), 3.64 (s, 3H), 5.17 (s, 2H), 6.80-6.85 (m, 2H), 7.24-7.28 (m, 1H), 7.31-7.41 (m, 3H), 7.47-7.50 (m, 2H). APCI [M+1]: 327.2.

### Step Six: 3-[2-(Dimethylamino-ethyl)]-5-fluoro-1-methyl-1H-indol-6-ol (82)

Following the procedure used to prepare compound 13, compound 79 gave the product compound 82 in 86% yield as a white solid, mp: 165-169° C. ¹H NMR (CDCl₃, 300 MHz) δ 2.36 (s, 6H), 2.63-2.69 (m, 2H), 2.82-2.88 (m, 2H), 3.56 (s, 3H), 6.48 (d, 1H, J=7.4), 6.73 (s, 1H), 7.16 (d, 1H, J=11.3). APCI [M+1]: 237.2. Elemental analysis: Calc.: C, 66.08; H, 7.25; N, 11.86. Found: C, 66.01; H, 7.22; N, 11.70.

### Example 34

### FIG. 9

### 3-[2-(Dimethylamino-ethyl)]-1-ethyl-5-fluoro-1H-indol-6-ol (Compound 83)

### Step One: 2-(6-Benzyloxy-1-ethyl-5-fluoro-1H-indol-3-yl)-N,N-dimethylethanamine (Compound 80)

Following the procedure used to prepare compound 5, compound 78 gave compound 80 in 61% yield as brown oil. ¹H NMR (CDCl₃, 300 MHz) δ 1.37 (t, 3H, J=7.1), 2.34 (s, 6H), 2.55-2.63 (m, 2H), 2.82-2.88 (m, 2H), 4.00 (q, 2H, J=7.1), 5.16 (s, 2H), 6.85 (d, 1H, J=6.9), 6.87 (s, 1H), 7.26 (d, 1H, J=11.6 Hz), 7.30-7.42 (m, 3H), 7.47-7.50 (m, 2H). APCI [M+1]: 341.2.

### Step Two: 3-[2-(Dimethylamino-ethyl)]-1-ethyl-5-fluoro-1H-indol-6-ol (Compound 83)

Following the procedure used to prepare compound 14, compound 80 gave compound 83 in 93% yield as a white solid, mp: 150-152° C. ¹H NMR (CDCl₃, 300 MHz) δ 1.36 (t, 3H, J=7.1), 2.36 (s, 6H), 2.62-2.68 (m, 2H), 2.83-2.89 (m, 2H), 3.94 (q, 2H, J=7.1), 6.69 (d, 1H, J=7.4), 6.80 (s, 1H), 7.18 (d, 1H, J=11.3). APCI [M+1]: 250.2. Elemental analysis C₁₄H₁₉FN₂O+0.2H₂O: Calc.: C, 66.21; H, 7.70; N, 11.03. Found: C, 66.21; H, 7.69; N, 10.78.

### Example 35

### FIG. 9

### 3-[2-(Dimethylamino-ethyl)]-5-fluoro-1-propyl-1H-indol-6-ol (Compound 84)

### Step One: 2-(6-Benzyloxy-5-fluoro-1-propyl-1H-indol-3-yl)-N,N-dimethylethanamine (Compound 81)

Following the procedure used to prepare compound 6, compound 78 gave the product compound 81 in 91% yield as a brown oil. ¹H NMR (CDCl₃, 300 MHz) δ 0.87 (t, 3H, J=7.4), 1.68-1.80 (m, 2H), 2.32 (s, 6H), 2.54-2.60 (m, 2H), 2.80-2.86 (m, 2H), 3.90 (t, 2H, J=7.1), 5.16 (s, 2H), 6.81-6.86 (m, 2H), 7.23-7.7.40 (m, 4H), 7.46-7.49 (m, 2H). APCI [M+1]: 355.2.

### Step Two: 3-[2-(Dimethylamino-ethyl)]-5-fluoro-1-propyl-1H-indol-6-ol (Compound 84)

Following the procedure used to prepare compound 15, compound 81 gave the product compound 84 in 88% yield as a white solid, mp: 157-159° C. ¹H NMR (CDCl₃, 300 MHz) δ 0.89 (t, 3H, J=7.4), 1.72-1.85 (m, 2H), 2.35 (s, 6H), 2.59-2.65 (m, 2H), 2.82-2.88 (m, 2H), 3.89 (t, 2H, J=7.1), 6.80 (d, 1H, J=7.7), 6.82 (s, 1H), 7.21 (d, 1H, J=11.0). APCI [M+1]: 265.2. Elemental analysis: Calc.: C, 68.16; H, 8.01; N, 10.60. Found: C, 67.87; H, 8.05; N, 10.43.

### Example 36

### Calcium Flux Assays

HEK 293 cells stably expressing the human 5-HT_{2A}, 5-HT_{2B}, or 5-HT_{2C} receptor were incubated for 20 h in serum-free DMEM containing 50 U/ml penicillin in tissue culture-treated black clear-bottom 384-well plates (Greiner, Germany) that were coated with 50 mg/l poly-L-lysine (Sigma, P-1524) in PBS. The cells were preincubated with 20 μl of reconstituted calcium dye (Calcium Plus Assay Kit, Molecular Devices) for 75 min at 37° C. in a humidified incubator in assay buffer (Hanks balanced salt solution (HBSS), 50 mM HEPES, 2.5 mM probenecid, 100 mg/l ascorbic acid, pH 7.4). The plates were allowed to cool to room temperature for 10 min and were transferred to a FLIPR Tetra fluorescence image plate reader (Molecular Devices). 20 μl of the test compounds in assay buffer was automatically added and fluorescence was measured for 60 s. The baseline was averaged from the data points immediately before the additions and results were exported as the maximal response over baseline during 60 s after addition. Each compound was measured at seven concentrations from 10 μM to 10 pM in triplicate. The data were analyzed in Prism (Graphpad). In dephosphorylation experiments, a 20 μM drug solution was incubated at 37° C. for 90 min with 20 U/ml calf alkaline phosphatase (New England BioLabs).

### Table 1

Functional activity in an intracellular calcium flux assay using cells stably expressing 5-HT_{2A}, 5-HT_{2B}, or 5-HT_{2C} receptors. Shown is the mean EC₅₀±SD and the efficacy as the percentage of the maximal 5-HT response±SD of at least three independent experiments, each performed in triplicate.

TABLE 1

| Compound | 5-HT_{2A} | | 5-HT_{2B} | | 5-HT_{2C} | |
|---|---|---|---|---|---|---|
| 5-HT | 9.4 ± 1.38 | (100%) | 0.96 ± 0.08 | (100%) | 0.11 ± 0.01 | (100%) |
| 22 | >10,000 | (21 ± 6 @ 10,000) | N/A | | 9.4 ± 4.9 | (82 ± 3%) |
| 23 | >10,000 | (17 ± 3.7 @ 10,000) | N/A | | 13 ± 1.74 | (71 ± 2.9%) |
| 24 | >2,000 | (21 ± 5.5 @ 10,000) | >5,000 | (14 ± 4.8 @ 10,000) | 57 ± 17.1 | (77 ± 4.4%) |
| 25 | N/A | | 1,453 ± 197 | (31 ± 7.5%) | 168 ± 27.2 | (75 ± 4.9%) |
| 26 | 1,490 ± 747 | (25 ± 4.1%) | 217 ± 95.9 | (28 ± 3.9%) | 43 ± 18.8 | (82 ± 3.0%) |
| 27 | N/A | | 334 ± 88.7 | (54 ± 9.5%) | 96 ± 19.8 | (75 ± 2.6%) |
| 28 | N/A | | N/A | | 76 ± 39.1 | (45 ± 5.0%) |
| 29 | N/A | | N/A | | 346 ± 97.0 | (43 ± 9.3%) |
| 30 | N/A | | N/A | | 858 ± 289 | (37 ± 3.6%) |
| 31 | >10,000 | (21 ± 7.9 @ 10,000) | 55 ± 7.77 | (51 ± 0.7%) | 9.9 ± 3.27 | (87 ± 9.3%) |
| 32 | >10,000 | (26 ± 7.6 @ 10,000) | 166 ± 113 | (66 ± 0.8%) | 31 ± 23.6 | (85 ± 6.6%) |
| 33 | N/A | | 182 ± 102 | (78 ± 1.2%) | 384 ± 193 | (91 ± 3.4%) |
| 34 | N/A | | 397 ± 133 | (21 ± 0.6%) | 24 ± 8.38 | (82 ± 9.0%) |
| 35 | >10,000 | (14 ± 4.8 @ 10,000) | 777 ± 383 | (47 ± 1.7%) | 246 ± 185 | (88 ± 4.2%) |

| 36 | N/A | 543 ± 325 | (64 ± 2.,6%) | 659 ± 352 | (87 ± 7.1%) |
|---|---|---|---|---|---|
| 37 | N/A | N/A | | 354 ± 195 | (69 ± 14.7%) |
| 38 | N/A | N/A | | 1,254 ± 534 | (80 ± 6.2%) |
| 39 | N/A | 3,122 ± 1,175 | (26 ± 5.9%) | 762 ± 469 | (65 ± 14.1%) |

| | | | | |
|---|---|---|---|---|
| 40 | N/A | N/A | 3.1 ± 1.20 | (72 ± 12.4%) |
| 41 | N/A | N/A | 1.4 ± 0.19 | (37 ± 2.9%) |
| 42 | N/A | N/A | 741 | (34%)^{a} |
| 50 | N/A | N/A | 282 | (81%)^{a} |
| 51 | N/A | N/A | 233 | (99%)^{a} |
| 52 | N/A | N/A | 44 | (79%)^{a} |
| 58 | N/A | N/A | 2.4 ± 0.44 | (51 ± 3.3%) |
| 59 | N/A | N/A | 0.82 ± 0.23 | (51 ± 4%) |
| 60 | N/A | N/A | 4.2 ± 0.94 | (69 ± 2.8%) |
| 61 | N/A | N/A | 13 ± 1.51 | (38 ± 3.6%) |
| 65 | N/A | N/A | 16 ± 3.46 | (41 ± 1.4%) |
| 73 | N/A | N/A | 274 ± 57.7 | (48 ± 2.3%) |
| 82 | N/A | N/A | 589 ± 546 | (83 ± 8%) |
| 83 | N/A | N/A | 221 ± 27 | (73 ± 4%) |
| 84 | N/A | N/A | 507 ± 314 | (70 ± 15%) |

N/A: <10% max HT @ 10,000 nM
^{a}These entries represent the results of single measurements.

### Example 37

### Appetite Suppression Studies in Mice

Mice were deprived of food, but not water, for 18 hours. They were then injected i.p. with test compounds dissolved in 0.9% NaCl containing 1 mg per ml ascorbic acid for protection against oxidation. Each mouse was placed in an individual cage for 30 minutes and then presented with a small petri dish containing a gel made from gelatin, powdered milk and sucrose. The dish was weighed at zero time and at 15-minute intervals for the next hour in order to quantitate food consumption. Controls were injected with saline-ascorbic acid; ±fenfluramine was used as an active control. The results are shown in Table 2.

TABLE 2

| | | | Mean food | Mean food | |
|---|---|---|---|---|---|
| | | | consumption | consumption | |
| | | | 60 min. after | 60 min. after | |
| | Dosage | | injection: g (±SEM) | injection: g (±SEM) | % Reduction |
| Substance | (mg/kg) | N | Saline Control | Compound | from control |
| (±)-Fenfluramine | 5 | 9 | 1.47 (.15) | 0.90 (.04) | 39 |
| | 10 | 10 | 1.33 (.09) | 0.41 (.11) | 69 |
| compound 22 | 10 | 10 | 0.66 (0.2) | 0.15 (0.1) | 77 |

p value <0.05 for compound versus saline control

### Example 38

### Animal Model Studies for OCD

Serotonin produces an itch sensation when applied to the human skin and has been suggested to be involved in pruritic diseases. Further research demonstrates that an subcutaneous (SC) injection of 5-HT into the rostral back of the mouse elicits scratching with the hind paws, which is itch-associated rather than a pain response (Kuraishi, Y., Nagasawa, T., Hayashi, K., Satoh, M. “Scratching Behavior Induced by Pruritogenic but not Algesiogenic Agents in Mice” Eur. J. Pharmacol. 1995, 275, 229). The 5-HT action is at least partly mediated by 5-HT₂ receptors in the skin, as shown by blocking with specific antagonists (Yamaguchi, T., Nagasawa, T., Satoh, M., Kuraishi, Y. “Itch-associated Response Induced by Intradermal Serotonin Through 5-HT2 Receptors in Mice” Neurosci. Res. 1999, 35, 77). The effect of test compounds on itch-associated scratching in the mice may indicate their action on 5-HT receptors, and this study was carried out as an animal model for OCD. The subjects were male Swiss-Webster mice, 4-6 weeks old, weighing 25-45 g. Mice were housed 5 per cage, given free access to standard mouse food and water except during experiments, and maintained in a temperature-controlled room (70° F.). Serotonin and all test drugs were made up with ascorbic acid to protect against oxidation. Two mice, one a control, the other experimental, were tested each time. Each mouse was separately placed into a plexiglas box.

Mice were injected subcutaneously between the shoulder blades with 0.1 ml of serotonin, 0.4 mg per ml in 0.15 M saline plus ascorbic acid, 1 mg/ml. Test compounds were injected i.p. 5 minutes before the inducer. The cumulative number of scratches with a hind leg was recorded at 5 minute intervals for 30 minutes. One saline injected control and one test animal were tested together in each assay. The results are shown in Table 3. Testing was also carried out by oral gavage as shown in Table 4.

TABLE 3

| | | | | Average Number | |
|---|---|---|---|---|---|
| | | | Average Number of | of Control | |
| | | | Scratches 30 min. | Scratches 30 min. | |
| | Dose | | after injection | after injection | % Reduction |
| Compound | (mg/kg) | N | (±SEM) | (±SEM) | from control |
| Fluoxetine | 10 | 5 | 154.8 ± 105.2 | 424.6 ± 146.1 | 63.5 |
| (±)-Fenfluramine | 3 | 5 | 22.8 ± 11.7 | 459.6 ± 104.5 | 95 |
| compound 22 | 10 | 11 | 4.7 ± 4.7 | 321.1 ± 84.5 | 98.5 |
| compound 40 | 3 | 5 | 8.2 ± 5.1 | 246 ± 107.5 | 97* |

p value <0.05 for compound versus saline control (*p = 0.09)

TABLE 4

| | | | | Average | |
|---|---|---|---|---|---|
| | | | Average | Number of | |
| | | | Number of | Control | |
| | | | Scratches | Scratches | |
| | | | 30 min. | 30 min. | |
| | | | after | after | |
| | Dose | | injection | injection | % Reduction |
| Compound | (mg/kg) | N | (±SEM) | (±SEM) | from control |
| (±)-Fenfluramine | 10 | 7 | 35 ± 25.8 | 595 ± 142 | 94 |
| compound 22 | 20 | 7 | 102 ± 50.8 | 595 ± 142 | 83 |

p < 0.05

The results demonstrate that the number of scratches was decreased when mice were treated with certain compounds of the invention, compared to control animals.

### Example 39

### Schizophrenia and Psychosis

Over 2.2 million people are currently suffering from schizophrenia in the US. Current therapies for schizophrenia are mixed D2/5-HT_{2A} antagonists (“atypical antipsychotics). However these medicines can have problematic side effects and are not always effective. 5-HT_{2C} agonists represent a novel approach to the treatment of schizophrenia and psychosis. Researchers at Wyeth have disclosed certain 5-HT_{2C} agonists that may be effective agents (Ramamoorthy, P. “[1,4]Diazepino[6,7-ij]quinoline derivatives as antipsychotic and antiobesity agents” U.S. Patent Application US2004/0009970 A1, Jan. 15, 2004). One of these 5-HT_{2C} agonists, WAY-163909 (vabicaserin), is currently under clinical development as a treatment for schizophrenia (Dunlop, J., Marquis, K., Lim, H., Leung, L., Kao, J., Cheesman, C., Rosenzweig-Lipson, S. “Pharmacological Profile of the 5-HT_{2C} Receptor Agonist WAY-163909; Therapeutic Potential in Multiple Indications” CNS Drug Reviews, 2006, 12, 167). Thus certain 5-HT_{2C} agonist compounds of the invention may also be useful as treatments for schizophrenia and psychosis.

Stereotypic climbing after treatment with apomorphine will be determined in mice following reported methods (Shuster, L.; Hudson, J.; Anton, M.; Righi, D. “Sensitization of Mice to Methylphenidate” Psychopharmacology 1982, 77, 31; Protais, P.; Constentin, J.; Schwartz, J. “Climbing Behavior Induced by Apomorphine in Mice: A Simple Test for the Study of Dopamine Receptors in Striatum” Psychopharmacology 1976, 50, 1 as follows. Mice are placed in a covered 22 cm square wire basket for 10 minutes. They are injected IP with test compounds or saline vehicle and then with apomorphine. Each mouse is scored every two minutes for a total of 60 minutes. The values used for scoring are: all four feet on the floor of the basket, 0; two feet on the floor, 1; all four feet clinging to the side of the basket, 2. Climbing usually begins within 4 minutes from injection of apomorphine and persists for at least 60 minutes after a dose of 8 mg/kg. Compounds that cause a reduction in the score values may be useful as treatments for schizophrenia and psychosis.

### Example 40

### Anxiety and Depression

Marble burying is an effective method for studying anxiety in mice (Njung'e, K; Handley, S. “Evaluation of Marble Burying as a Model of Anxiety” Pharmacol-Biochem-Behav. 1991, 38, 63), and the compounds of this invention will be evaluated in this assay. Twenty marbles are evenly spaced in a cage upon 5 cm of bedding. Swiss-Webster mice are treated either with a test compound or with saline vehicle, and then given 30 minutes in the cage with no prior training. At the end of 30 minutes, the number of marbles buried is counted. A higher number of marbles buried is associated with higher levels of anxiety. Test compounds that reduce marble burying may be useful as treatments for anxiety.

Compounds of the invention will also be evaluated using the elevated plus-maze. This is an assay of fear and anxiety in which a test animal is placed in the center of an elevated 4-arm maze in which 2 arms are open and 2 arms are closed. Using a video camera, the test animal's behavior is recorded by a blinded research observer. This test measures the degree to which the test animal avoids the unenclosed arms of the maze, a potentially dangerous environment. Test compounds that result in a larger amount of time spent in the open arms may be useful as treatments for anxiety.

### Example 41

### Diabetes

The compounds of this invention will be assayed for their ability to reduce plasma insulin levels (Zhou, L., Sutton, G., Rochford, J., Semple, R., Lam, D., Oksanen, L., Thornton-Jones, Z., Clifton, P., Yueh, C.-Y., Evans, M., McCrimmon, R., Elmquist, J., Butler, A, Heisler, L. “Serotonin 2C Receptor Agonists Improve Type 2 Diabetes via Melanocortin-4 Receptor Signaling Pathways” Cell Metabolism, 2007, 6, 398). Diet-induced obese mice will be treated with saline or a subanorectic dose of test compounds. Fasting plasma insulin and blood glucose levels will be determined 2 days before pump implantation and after 14 days of treatment with saline or test compounds. Compound-treated mice that display reduced plasma insulin levels relative to control mice, especially without altering blood glucose, food intake, or body weight may be useful as treatments for diabetes.

The disclosures of each and every patent, patent application and publication cited herein are hereby incorporated herein by reference in their entirety.

Although the invention has been disclosed with reference to specific embodiments, it is apparent that other embodiments and variations of the invention may be devised by others skilled in the art without departing from the true spirit and scope of the invention. The claims are intended to be construed to include all such embodiments and equivalent variations.

1. A compound represented by the structural formula I: 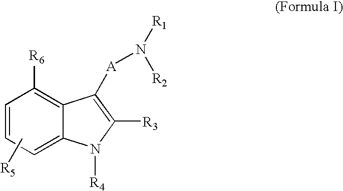
in which
A is C₁-C₄ alkylene, C₂-C₄ alkenylene, or C₂-C₄ alkynylene;
R₁ and R₂ are, independently for each occurrence, H, C₁-C₈ alkyl, C₂-C₈ alkenyl, or C₂-C₈ alkynyl, wherein the C₁-C₈ alkyl, C₂-C₈ alkenyl, or C₂-C₈ alkynyl are optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH;
R₃ is H, C₁-C₈ alkyl, C₂-C₈ alkenyl, or C₂-C₈ alkynyl, wherein the C₁-C₈ alkyl, C₂-C₈ alkenyl, or C₂-C₈ alkynyl are optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH; or R₃ is selected from the group consisting of halogen, C₁-C₈ alkylsulfonyl, formyl, COOH, hydroxy, C₁-C₈ alkoxyl, —SH, thio(C₂-C₈)alkyl, amino, C₁-C₈ alkylamino, and di(C₁-C₈ alkyl)amino;
R₄ is C₁-C₈ alkyl, C₂-C₈ alkenyl, or C₂-C₈ alkynyl, each of which is optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH; or R₄ is selected from the group consisting of C₁-C₈ alkylsulfonyl, formyl, hydroxy, C₁-C₈ alkoxyl, —SH, thio(C₂-C₈)alkyl, amino, C₁-C₈ alkylamino, and di(C₁-C₈ alkyl)amino;
R₅ represents 1-3 substituents, each of which is independently selected from the group consisting of C₁-C₈ alkyl, C₂-C₈ alkenyl, and C₂-C₈ alkynyl, each of which is optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, and di(C₁-C₈ alkyl)amino; or
R₅ represents 1-3 substituents, each of which is independently selected from the group consisting of halogen, C₁-C₈ alkylsulfonyl, formyl, COOH, hydroxy, C₁-C₈ alkoxyl, —SH, thio(C₂-C₈)alkyl, amino, C₁-C₈ alkylamino, and di(C₁-C₈ alkyl)amino;
R₆ is F or OR₇; and
R₇ is C₁-C₈ alkyl, optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH;
or a pharmaceutically acceptable salt thereof.

2. The compound of claim 1, wherein A is C₁-C₄ alkylene.

3. The compound of claim 2, wherein A is C₂ alkylene.

4. The compound of claim 1, wherein R₁ is hydrogen.

5. The compound of claim 1, wherein R₁ is C₁-C₈ alkyl.

6. The compound of claim 5, wherein R₁ is C₁ alkyl.

7. The compound of claim 5, wherein R₁ is C₂ alkyl.

8. The compound of claim 1, wherein R₁ is C₁-C₈ alkyl substituted with 1-3 substituents.

9. The compound of claim 8, wherein R₁ is C₁-C₈ alkyl substituted with 1-3 substituents, wherein at least one substituent is halogen (e.g., fluorine).

10. The compound of claim 8, wherein R₁ is C₂ alkyl substituted with 1-3 substituents.

11. The compound of claim 10, wherein R₁ is C₂ alkyl substituted with 1-3 halogens.

12. The compound of claim 11, wherein R₁ is C₂ alkyl substituted with 1-3 fluorines.

13. The compound of claim 12, wherein R₁ is C₂ alkyl substituted with 3 fluorines.

14. The compound of claim 13, wherein R₁ is —CH₂—CF₃.

15. The compound of claim 1, wherein R¹ is C₁-C₈ alkyl, C₂-C₈ alkenyl, or C₂-C₈ alkynyl, wherein each carbon of the C₁-C₈ alkyl, C₂-C₈ alkenyl, or C₂-C₈ alkynyl is substituted with no more than 2 substituents, each of which is independently selected from the group consisting of cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH.

16. The compound of claim 1, wherein R₂ is hydrogen.

17. The compound of claim 1, wherein R₂ is C₁-C₈ alkyl.

18. The compound of claim 17, wherein R₂ is C₁ alkyl.

19. The compound of claim 17, wherein R₂ is C₂ alkyl.

20. The compound of claim 1, wherein R₂ is C₁-C₈ alkyl substituted with 1-3 substituents.

21. The compound of claim 20, wherein R₂ is C₁-C₈ alkyl substituted with 1-3 substituents, wherein at least one substituent is halogen (e.g., fluorine).

22. The compound of claim 20, wherein R₂ is C₂ alkyl substituted with 1-3 substituents.

23. The compound of claim 22, wherein R₂ is C₂ alkyl substituted with 1-3 halogens.

24. The compound of claim 23, wherein R₂ is C₂ alkyl substituted with 1-3 fluorines.

25. The compound of claim 24, wherein R₂ is C₂ alkyl substituted with 3 fluorines.

26. The compound of claim 25, wherein R₂ is —CH₂—CF₃.

27. The compound of claim 1, wherein R² is C₁-C₈ alkyl, C₂-C₈ alkenyl, or C₂-C₈ alkynyl, wherein each carbon of the C₁-C₈ alkyl, C₂-C₈ alkenyl, or C₂-C₈ alkynyl is substituted with no more than 2 substituents, each of which is independently selected from the group consisting of cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH.

28. The compound of claim 1, wherein R₁ and R₂ are both C₁-C₈ alkyl.

29. The compound of claim 28, wherein R₁ and R₂ are both C₁ alkyl.

30. The compound of claim 28, wherein R₁ and R₂ are both C₂ alkyl.

31. The compound of claim 28, wherein R₁ is C₁ alkyl and R₂ is C₂ alkyl.

32. The compound of claim 1, wherein both R₁ and R₂ are C₁-C₈ alkyl substituted with 1-3 substituents.

33. The compound of claim 1, wherein both R₁ and R₂ are C₁-C₈ alkyl substituted with 1-3 substituents, wherein at least one substituent is halogen (e.g., fluorine).

34. The compound of claim 32, wherein both R₁ and R₂ are C₂ alkyl substituted with 1-3 substituents.

35. The compound of claim 34, wherein both R₁ and R₂ are C₂ alkyl substituted with 1-3 halogens.

36. The compound of claim 35, wherein both R₁ and R₂ are C₂ alkyl substituted with 1-3 fluorines.

37. The compound of claim 36, wherein both R₁ and R₂ are C₂ alkyl substituted with 3 fluorines.

38. The compound of claim 37, wherein both R₁ and R₂ are —CH₂—CF₃.

39. The compound of claim 1, wherein R₁ is hydrogen and R₂ is C₁-C₈ alkyl.

40. The compound of claim 39, wherein R₁ is hydrogen and R₂ is C₁ alkyl.

41. The compound of claim 39, wherein R₁ is hydrogen and R₂ is C₂ alkyl.

42. The compound of claim 1, wherein R₁ and R₂, together with the nitrogen to which they are attached, form a group selected from the following:

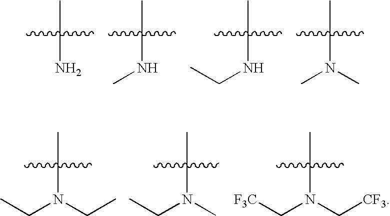

43. The compound of claim 1, wherein R₃ is hydrogen.

44. The compound of claim 1, wherein R³ is C₁-C₈ alkyl, C₂-C₈ alkenyl, or C₂-C₈ alkynyl, wherein each carbon of the C₁-C₈ alkyl, C₂-C₈ alkenyl, or C₂-C₈ alkynyl is substituted with no more than 2 substituents, each of which is independently selected from the group consisting of cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH.

45. The compound of claim 1, wherein R₄ is C₁-C₈ alkyl, C₂-C₈ alkenyl, or C₂-C₈ alkynyl, each of which is optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH; or R₄ is selected from the group consisting of C₁-C₈ alkylsulfonyl, formyl, hydroxy, C₁-C₈ alkoxyl, and thio(C₂-C₈)alkyl.

46. The compound of claim 1, wherein R⁴ is C₁-C₈ alkyl, C₂-C₈ alkenyl, or C₂-C₈ alkynyl, wherein each carbon of the C₁-C₈ alkyl, C₂-C₈ alkenyl, or C₂-C₈ alkynyl is substituted with no more than 2 substituents, each of which is independently selected from the group consisting of cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH.

47. The compound of claim 1, wherein R₄ is C₁-C₈ alkyl.

48. The compound of claim 47, wherein R₄ is C₁ alkyl.

49. The compound of claim 47, wherein R₄ is C₂ alkyl.

50. The compound of claim 47, wherein R₄ is C₃ alkyl.

51. The compound of claim 47, wherein R₄ is C₄ alkyl.

52. The compound of claim 47, wherein R₄ is cycloalkyl.

53. The compound of claim 52, wherein R₄ is C₃ cycloalkyl.

54. The compound of claim 52, wherein R₄ is C₄ cycloalkyl.

55. The compound of claim 52, wherein R₄ is C₅ cycloalkyl.

56. The compound of claim 52, wherein R₄ is C₆ cycloalkyl.

57. The compound of claim 52, wherein R₄ is C₇ cycloalkyl.

58. The compound of claim 47, wherein R₄ is cycloalkyl substituted alkyl.

59. The compound of claim 58, wherein R₄ is cycloalkyl substituted C₁-C₄ alkyl.

60. The compound of claim 59, wherein R₄ is cycloalkyl substituted C₁ alkyl.

61. The compound of claim 59, wherein R₄ is cycloalkyl substituted C₂ alkyl.

62. The compound of claim 59, wherein R₄ is cycloalkyl substituted C₃ alkyl.

63. The compound of claim 59, wherein R₄ is cycloalkyl substituted C₄ alkyl.

64. The compound of claim 1, wherein R₅ represents 1 substituent.

65. The compound of claim 64, wherein the compound is of the following formula:

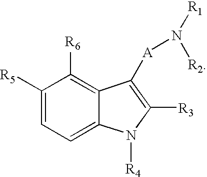

66. The compound of claim 65, wherein R₅ is halogen.

67. The compound of claim 66, wherein R₅ is fluorine.

68. The compound of claim 64, wherein the compound is of the following formula:

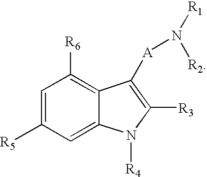

69. The compound of claim 68, wherein R₅ is halogen.

70. The compound of claim 69, wherein R₅ is fluorine.

71. The compound of claim 64, wherein the compound is of the following formula:

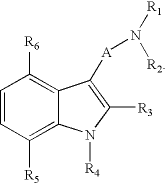

72. The compound of any claim 71, wherein R₅ is halogen.

73. The compound of any claim 72, wherein R₅ is fluorine.

74. The compound of claim 1, wherein R₅ represents 2 substituents.

75. The compound of claim 74, wherein the compound is of the following formula:

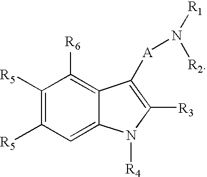

76. The compound of claim 75, wherein both R₅ substituents are halogen.

77. The compound of claim 76, wherein both R₅ substituents are fluorine.

78. The compound of claim 74, wherein the compound is of the following formula:

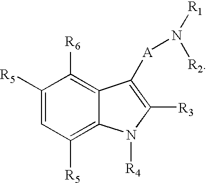

79. The compound of claim 78, wherein both R₅ substituents are halogen.

80. The compound of claim 79, wherein both R₅ substituents are fluorine.

81. The compound of claim 74, wherein the compound is of the following formula:

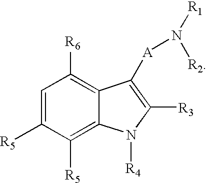

82. The compound of claim 81, wherein both R₅ substituents are halogen.

83. The compound of claim 82, wherein both R₅ substituents are fluorine.

84. The compound of claim 1, wherein R⁵ represents 1-3 substituents, each of which is independently selected from the group consisting of C₁-C₈ alkyl, C₂-C₈ alkenyl, or C₂-C₈ alkynyl, wherein each carbon of the C₁-C₈ alkyl, C₂-C₈ alkenyl, or C₂-C₈ alkynyl is substituted with no more than 2 substituents, each of which is independently selected from the group consisting of cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH.

85. The compound of claim 1, wherein R₆ is fluorine.

86. The compound of claim 1, wherein R₆ is OR₇.

87. The compound of claim 86, wherein R₇ is C₁-C₈ alkyl.

88. The compound of claim 87, wherein R₇ is C₁ alkyl.

89. The compound of claim 86, wherein R₇ is C₁ alkyl substituted with 1-3 substituents.

90. The compound of claim 89, wherein R₇ is C₁ alkyl substituted with 1-3 halogens.

91. The compound of claim 90, wherein R₇ is C₁ alkyl substituted with 1-3 fluorines.

92. The compound of claim 91, wherein R₇ is C₁ alkyl substituted with 2 fluorines.

93. The compound of claim 91, wherein R₇ is C₁ alkyl substituted with 3 fluorines.

94. The compound of claim 87, wherein R₇ is C₂ alkyl.

95. The compound of claim 86, wherein R₇ is C₂ alkyl substituted with 1-3 substituents.

96. The compound of claim 95, wherein R₇ is C₂ alkyl substituted with 1 substituent.

97. The compound of claim 96, wherein R₇ is —CH₂—CH₂—OH.

98. The compound of claim 96, wherein R₇ is —CH₂—CH₂—(C₁-C₈ alkoxyl).

99. The compound of claim 98, wherein R₇ is —CH₂—CH₂—O—CH₃.

100. The compound of claim 96, wherein R₇ is —CH₂—CH₂— (di(C₁-C₈ alkyl)amino).

101. The compound of claim 100, wherein R₇ is —CH₂—CH₂—N(CH₃)₂.

102. The compound of claim 87, wherein R₇ is C₃ alkyl.

103. The compound of claim 87, wherein R₇ is cycloalkyl.

104. The compound of claim 103, wherein R₇ is C₃ cycloalkyl.

105. The compound of claim 103, wherein R₇ is C₄ cycloalkyl.

106. The compound of claim 103, wherein R₇ is C₅ cycloalkyl.

107. The compound of claim 103, wherein R₇ is C₆ cycloalkyl.

108. The compound of claim 103, wherein R₇ is C₇ cycloalkyl.

109. The compound of claim 87, wherein R₇ is cycloalkyl substituted alkyl.

110. The compound of claim 109, wherein R₇ is cycloalkyl substituted C₁-C₄ alkyl.

111. The compound of claim 109, wherein R₇ is cycloalkyl substituted C₁ alkyl.

112. The compound of claim 109, wherein R₇ is cycloalkyl substituted C₂ alkyl.

113. The compound of claim 109, wherein R₇ is cycloalkyl substituted C₃ alkyl.

114. The compound of claim 109, wherein R₇ is cycloalkyl substituted C₄ alkyl.

115. The compound of claim 86, wherein R⁷ is C₁-C₈ alkyl, wherein each carbon of the C₁-C₈ alkyl is substituted with no more than 2 substituents, each of which is independently selected from the group consisting of cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH.

116. A pharmaceutical composition comprising a compound of claim 1, formula I.

117. A dosage form comprising a compound of claim 1, formula I.

118. The dosage form of claim 117, wherein the dosage form is an oral dosage form.

119-120. (canceled)

121. A method for suppressing appetite in a subject, the method comprising administering to the subject a compound of claim 1, formula I, such that appetite is suppressed in the subject.

122-540. (canceled)

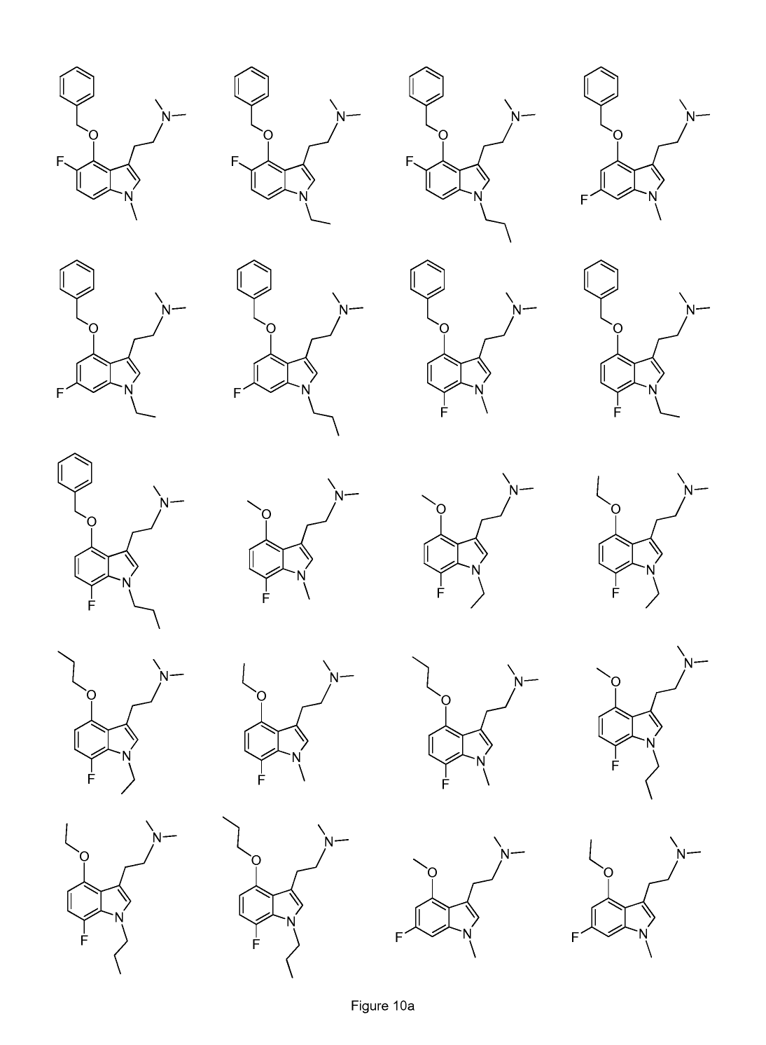

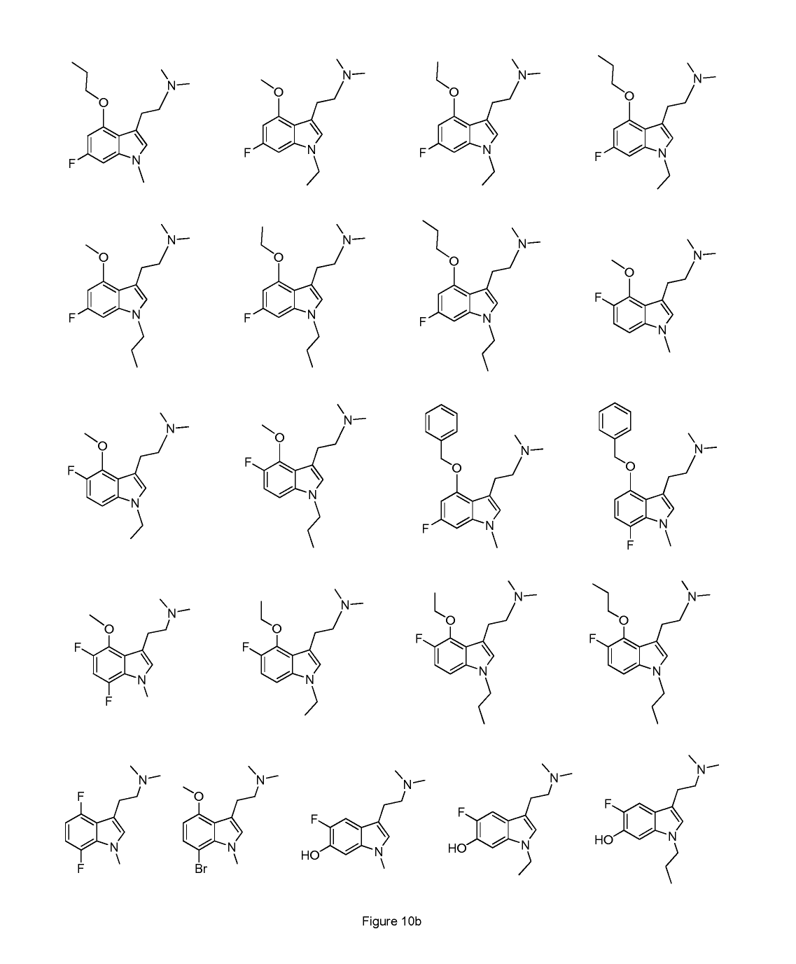

541. A compound selected from the compounds disclosed in FIG. 10a or 10b, or a pharmaceutically acceptable salt thereof.

542. The compound of claim 541, wherein the compound is an HCl salt.
